(19) United States
(12) Patent Application Publication
(10) Pub. No.: US 2025/0197803 A1
(43) Pub. Date: Jun. 19, 2025

(54) A METHOD OF DIFFERENTIATING AN INDUCED PLURIPOTENT STEM CELL INTO A RETINAL PIGMENT EPITHELIAL CELL, A RETINAL PIGMENT EPITHELIAL CELL AND METHODS OF USING THE RETINAL PIGMENT EPITHELIAL CELL

(71) Applicants: Agency for Science, Technology and Research, Singapore (SG); CellResearch Corporation Pte. Ltd, Singapore (SG); Singapore Health Services Pte Ltd, Singapore (SG)

(72) Inventors: Xinyi SU, Singapore (SG); Regha KAKKAD, Singapore (SG); Mayuri BHARGAVA, Singapore (SG); Binxia YANG, Singapore (SG); Walter HUNZIKER; Zengping LIU, Singapore (SG); Haofei WANG, Singapore (SG); Bhav Harshad PARIKH, Singapore (SG); Qingfeng CHEN, Singapore (SG); Fritz Shien Choong LAI, Singapore (SG); Chou CHAI, Singapore (SG); Kah Leong LIM, Singapore (SG); Toan Thang PHAN

(73) Assignees: Agency for Science, Technology and Research, Singaore (SG); CellResearch Corporation Pte. Ltd, Singapore (SG); Singapore Health Services Pte Ltd, Singapore (SG)

(21) Appl. No.: 18/833,821

(22) PCT Filed: Jan. 27, 2023
(86) PCT No.: PCT/SG2023/050051

§ 371 Date: Jul. 26, 2024

Related U.S. Application Data
(60) Provisional application No. 63/303,849, filed on Jan. 27, 2022.

(51) Int. Cl. C12N 5/079; A61K 35/30

(52) U.S. Cl. CPC C12N 5/0621; A61K 35/30; C12N 2500/05; C12N 2500/32; C12N 2500/38; C12N 2501/105; C12N 2501/115; C12N 2501/16; C12N 2501/415; C12N 2506/45

(57) ABSTRACT

The present invention relates to a method of differentiating an induced pluripotent stem cell into a retinal pigment epithelial cell. Additionally, the present invention relates to a retinal pigment epithelial cell culture obtainable by the differentiation method and a retinal pigment epithelial cell culture obtained by the differentiation method. In addition, the present invention concerns a retinal pigment epithelium consisting of or comprising a retinal pigment epithelial cell culture obtainable or obtained by the differentiation method. The present invention also relates to a pharmaceutical composition comprising a retinal pigment epithelial cell culture obtained by the differentiation method. The present invention concerns a method of treating a retinal degenerative disease in a subject, comprising administering to a subject a retinal pigment epithelial cell differentiated from the induced pluripotent stem cell by the method. Finally, the present invention also refers to an in vivo method of detecting the survival rate of a retinal pigment epithelial cell differentiated from an induced pluripotent stem cell by the defined method in a subject and an in vitro method of determining the immunogenicity of said retinal pigment epithelial cell differentiated from an induced pluripotent stem cell by the defined method in said subject, to whom said differentiated RPE cell has been pre-delivered.

### CROSS-REFERENCE TO RELATED APPLICATIONS

The present application claims the benefit of priority of U.S. Provisional Patent Application No. 63/303,849 filed 27 Jan. 2022, the content of which is hereby incorporated by reference in its entirety for all purposes.

### BACKGROUND OF THE INVENTION

The present invention relates to a method of generating an induced pluripotent stem cell. In addition, the present invention concerns an induced pluripotent stem cell population obtainable by the method and an induced pluripotent stem cell population obtained by the method. The present invention also relates to a pharmaceutical composition comprising the induced pluripotent stem cell of the present invention. The present invention also relates to a method of differentiating the induced pluripotent stem cell of this invention. In addition, a pharmaceutical composition comprising a differentiated induced pluripotent stem cell obtained by the method is also concerned. Further, the present invention concerns a method of treating a congenital or acquired degenerative disorder in a subject, comprising administering to a subject a target cell differentiated from pluripotent stem cell. The present invention also relates to a method of differentiating said induced pluripotent stem cell into a retinal pigment epithelial cell. Additionally, the present invention relates to a retinal pigment epithelial cell culture obtainable by the differentiation method and a retinal pigment epithelial cell culture obtained by the differentiation method. In addition, the present invention concerns a retinal pigment epithelium consisting of or comprising a retinal pigment epithelial cell culture obtainable or obtained by the differentiation method. The present invention also relates to a pharmaceutical composition comprising a retinal pigment epithelial cell culture obtained by the differentiation method. The present invention concerns a method of treating a retinal degenerative disease in a subject, comprising administering to a subject a retinal pigment epithelial cell differentiated from the induced pluripotent stem cell by the method. Finally, the present invention also refers to an in vivo method of detecting the survival rate of a retinal pigment epithelial cell differentiated from an induced pluripotent stem cell by the defined method in a subject and an in vitro method of determining the immunogenicity of said retinal pigment epithelial cell differentiated from an induced pluripotent stem cell by the defined method in said subject, to whom said differentiated RPE cell has been pre-delivered.

### FIELD OF THE INVENTION

Stem cells are a cell population possessing the capacities to self-renew indefinitely and to differentiate in multiple cell or tissue types. The ability of stem cells to self-renew is critical to their function as reservoir of primitive undifferentiated cells and the “plasticity” of stem cells relies on their ability to trans-differentiate into tissues different from their origin and, perhaps, across embryonic germ layers. In contrast, most somatic cells have a limited capacity for self-renewal due to telomere shortening (reviewed, for example, in Dice, J. F. (1993) Physiol. Rev. 73, 149-159). Stem cell-based therapies thus have the potential to be useful for the treatment of a multitude of human and animal diseases.

Embryonic stem cells (from approximately days 3 to 5 after fertilization) proliferate indefinitely and can differentiate spontaneously into all tissue types: they are thus termed pluripotent stem cells (reviewed, for example, in Smith, A. G. (2001) Annu. Rev. Cell. Dev. Biol. 17, 435-462). Even though the potential of embryonic stem cells is enormous, their use implies many ethical problems. Therefore, non-embryonic stem cells have been proposed as alternative sources.

Adult stem cells are more tissue-specific and may have less replicative capacity: they are thus termed multipotent stem cells (reviewed, for example, in Paul, G. et al. (2002) Drug Discov. Today 7, 295-302). These cells can be derived from the bone marrow stroma, fat tissue and dermis and have the ability to differentiate inter alia into chondrocytes, adipocytes, osteoblasts, myoblasts, cardiomyocytes, astrocytes, and tenocytes. In many cases, however, the number of stem cells extracted from the bone marrow stroma, fat tissue, dermis and umbilical cord blood is rather low.

A comprehensive source for very young and adaptable adult stem cells, also referred to as neonatal stem cells, is the umbilical cord blood or tissue or the placenta. For example, a large amount of stem cells can be derived from umbilical cord tissue, namely from Wharton's jelly, the matrix of umbilical cord (Mitchell, K. E. et al. (2003) Stem Cells 21, 50-60; U.S. Pat. No. 5,919,702; US Patent Application 2004/0136967). These cells have been shown to have the capacity to differentiate, for example, into a neuronal phenotype and into cartilage tissue, respectively. Mesenchymal stem cells have also been isolated from the subendothelial layer of the umbilical cord vein, one of the three vessels (two arteries, one vein) found within the umbilical cord (Romanov, Y. A. et al. (2003) Stem Cells 21, 105-110; Covas, D. T. et al. (2003) Braz. J. Med. Biol. Res. 36, 1179-1183). Further, mesenchymal stem cells as well as epithelial stem cells have successfully been isolated from the amniotic tissue of the umbilical cord (US2006/0078993). Although, for example, mesenchymal stem cells can undergo differentiation in vitro and in vivo, making these stem cells promising candidates for mesodermal defect repair and disease management, the use of adult stem cells is limited by their multipotency. To overcome this limitation, non-embryonic cells can be reprogrammed to pluripotent stem cells: the so called induced pluripotent stem cells (iPS).

IPS were generated for the first time by Takahashi and Yamanaka, who reprogrammed non-embryonic cells to a pluripotent state through overexpression of the four transcription factors OCT3/4, SOX2, KLF4 and C-MYC, also known as Yamanaka factors (Takahashi, K. and Yamanaka, S. (2006), Cell, 126(4), pp. 663-676). In detail, Takahashi and Yamanaka used mouse embryonic fibroblasts and introduced the Yamanaka factors via retroviral transduction, thereby allowing the overexpression of the transcription factors and thus generating cells exhibiting the morphology and growth properties of embryonic cells. Although this method was a major breakthrough, the transduction process may result in an incorporation of the transferred DNA into the genome of the host cells making the iPS critical for therapeutic treatment in humans. A non-integrative alternative to generate iPS has been established in 2011 by Okita, K. et al., Nature methods, 8(5), pp. 409-412. Okita et al. used electroporation to transfer three episomal plasmid vectors encoding the Yamanaka factors and a p53-shRNA for p53 suppression into human dermal fibroblasts and dental pulp, thus, allowing the overexpression of the exogenous DNA and thereby generating integration free human iPS. To support the growth and maintenance of the integration free human iPS, Okita et al., supra, cultivated the iPS on a feeder layer consisting of mouse embryonic fibroblast (MEF) or a STO cell line, which has been transformed with neomycin resistance and murine LIF genes (SNL). The cultivation on a feeder layer, however, may entail the risk of contaminating the iPS with foreign DNA. Thus, the insertion free iPS according to Okita et al., supra, may also be critical for therapeutic treatment in humans.

A decade after its conception, iPS technology has entered the clinical translation stage with first-in-human trials being conducted for age-related macular degeneration (AMD; Mandai, M., et al., N Engl J Med, 2017. 376(11): p. 1038-1046) and Parkinson's Disease (PD; Reardon, S. and Cyranoski, D. (2014) ‘Japan stem-cell trial stirs envy’, Nature. England, pp. 287-288. doi: 10.1038/513287a). The greatest promise of iPS technology lies in its potential for enabling autologous cell therapy, which may circumvent the need for long-term immunosuppression or histocompatibility matching to prevent rejection of transplanted cells. This paradigm has been demonstrated with fibroblasts and bone marrow derived iPS in non-human primate models (Morizane, A., et al., Stem Cell Reports, 2013. 1(4): p. 283-92; Hallett, P. J., et al., Cell Stem Cell, 2015. 16(3): p. 269-74; Wang, S., et al., Cell Discov, 2015. 1: p. 15012; Shiba, Y., et al., Nature, 2016. 538(7625): p. 388-391), and is the basis of the first human trial of iPS-based cell therapy for AMD (Mandai, M., et al., N Engl J Med, 2017. 376(11): p. 1038-1046). However, the significant period of time and costs associated with the production of clinical-grade iPS will make it unlikely to be implemented on a large-scale for human therapy. In addition, circumstances exist where the generation of autologous iPS from a patient may not be practical. For instance, for patients carrying disease-causing mutations, it is first necessary to correct these mutations before iPS derived from these patients can be used. This is achievable when the mutations are tractable but in cases where the mutations are intractable, such as those underlying the sporadic form of many diseases, a gene correction strategy may not be tenable.

Accordingly, there is still a need for an alternative method to generate iPS, wherein the resulting iPS is capable to differentiate into a target cell, such as retinal pigment epithelial (RPE) cells suitable for therapeutic treatment in humans.

Stem cells currently used for RPE generation, namely induced pluripotent stem cells (iPS) and embryonic stem cells (ES), have some disadvantages. It is well-established that genomes of individuals accumulate mutations over the lifespan, which underlies age-related diseases like cancer (Stratton M R et al., 2009, Nature 458, 719-7249 2001). iPS cells derived from younger individuals carry less mutations compared to those derived from elderly individuals. Comparing DNA sequence of subjects of age ranging from 21-100 years genetic and epigenetic mutation found in iPS cells increased with increasing age of the donor (Lo Sardo et at., 2017, Nat Biotechnol. 35(1):69-74). Age-related abnormalities increased in mitochondrial DNA as well, with fibroblast-derived iPS cells of elderly subjects harbouring significantly higher mutations than younger subjects (Kang et al., 2016, Cell Stem Cell 18, 625-636, May 5, 2016). iPS cells, derived from adults, may also require immune-suppression if derived from an allogenic host. The use of ES cells are associated with ethical issues and immune-rejection requiring use of immunosuppression. Skin cells are widely used for generation iPS cells due to ease of tissue collection, however they have higher changes of developing mutations due to long-term exposure to UV from sunlight (Apalla Z. et al., 2017, Dermatol Pract Concept. 2017 April; 7(2): 1-6).

Consequently, it is an object of the invention to provide a method of generating iPS cells and differentiating said particular iPS cells to RPE cells that meet these needs.

### SUMMARY OF THE INVENTION

The invention relates to a method of generating an induced pluripotent stem (iPS) cell as described herein, a resulting induced pluripotent stem cell, a method of differentiating a resulting induced pluripotent stem cell and a method of treating a disorder in a subject with a differentiated cell derived from an induced pluripotent stem cell.

Accordingly, the invention provides a method of generating an induced pluripotent stem cell, wherein the method comprises expressing exogenous nucleic acid encoding the proteins OCT3/4, SOX2, KLF4, LIN28 and L-MYC and the p53-shRNA in a stem cell of the amniotic membrane of the umbilical cord under conditions suitable to reprogram the stem cell, thereby generating the induced pluripotent stem cell. In embodiments of this method stem cell of the amniotic membrane of the umbilical cord is a mesenchymal stem cell of the amniotic membrane of the umbilical cord or an epithelial stem cell of the amniotic membrane of the umbilical cord.

Further, the invention also provides an induced pluripotent stem cell population obtainable by the method as well as an induced pluripotent stem cell population obtained by the method. The induced pluripotent stem cell population can either be an induced pluripotent stem cell population that is derived from a mesenchymal stem cell (population) of the amniotic membrane of umbilical cord or an induced pluripotent stem cell population that is derived from an epithelial stem cell (population) of the amniotic membrane of the umbilical cord.

Additionally, the invention also provides a pharmaceutical composition comprising an induced pluripotent stem cell of the present invention.

Further, the invention provides a method of differentiating an induced pluripotent stem cell of the present invention into a target cell, wherein the induced pluripotent stem cell is differentiated into the target cell under conditions suitable for differentiation. Consequently, the invention also provides a pharmaceutical composition comprising a differentiated induced pluripotent stem cell obtained by the present invention.

The invention also provides a method of treating a congenital or acquired degenerative disorder in a subject, comprising administering to a subject a target cell differentiated from a pluripotent stem cell obtained by the present invention.

Additionally, the invention provides an extracellular membranous vesicle produced by an induced pluripotent stem cell population of the invention or produced by a cell obtained by differentiation of an induced pluripotent stem cell of the invention. The invention further comprises the use of such an extracellular membranous vesicle of the invention as delivery carrier of a therapeutic agent.

The invention also provides a cell culture medium comprising Mammary Epithelial Basal Medium MCDB 170, EpiLife medium, DMEM (Dulbecco's modified eagle medium), F12 (Ham's F12 Medium) and FBS (Fetal Bovine Serum).

The invention further relates to a method of differentiating an induced pluripotent stem (iPS) cell as described herein into a retinal pigment epithelial (RPE) cell, a resulting RPE cell, a retinal pigment epithelium consisting of or comprising the RPE cell as described elsewhere herein, a method of treating a retinal degenerative disease in a subject with a RPE cell differentiated from an iPS cell by the method as described herein as well as a pharmaceutical composition comprising a RPE cell obtained by the method as described herein. Further, the invention also relates to an in vivo and an in vitro method using the RPE cell obtained by the method as described herein.

The inventors describe the use of iPS cells derived from umbilical cord lining cells (short: CLiPS) to generate RPE cells for clinical use. The inventors differentiated CLiPS to RPEs using the method of the present invention in comparion to ES and skin iPS cells and generated RPEs with consistently increased RPE differentiation efficiency. CLiPS-derived RPEs had higher pigmentation than ES-derived RPEs based on increased expression levels of pigmentation specific genes such as MITF, PMEL17, and TRYP2 and RPE-specific genes such as BEST1, RPE65, MERTK, RLBP1. Also on the functional level by comparing bioenergetics of RPE derived from different stem cells (CLiPS, ES and skin iPS), it was demonstrated that CLiPS-RPE comprise increased glycolytic and mitochondrial respiration levels compared to ES-derived RPEs.

Accordingly, in a first aspect of the invention, the invention provides a method of differentiating an iPS cell into a RPE cell, the method comprising culturing the iPS cell derived from a stem cell of the amniotic membrane of the umbilical cord in a differentiation medium under conditions suitable for the differentiation into a RPE cell, thereby differentiating the iPS cell into the RPE cell.

In a second aspect of the invention, the invention provides a RPE cell culture obtainable by the method as well as a RPE cell culture obtained by the method.

In a third aspect of the invention, the invention also provides a retinal pigment epithelium consisting of or comprising said RPE cell culture obtainable by the method as well as consisting of or comprising said RPE cell culture obtained by the method.

In a fourth aspect of the invention, the invention also provides a pharmaceutical composition comprising a RPE cell culture obtained by the method of the present invention.

In a fifth aspect of the invention, the invention also provides a method of treating a retinal degenerative disease in a subject, comprising administering to a subject a RPE cell differentiated from an iPS cell by the method of the present invention.

In a sixth aspect of the invention, the invention provides an in vivo method of detecting the survival rate of a RPE cell differentiated from an iPS cell by the method as defined herein in a subject, the method comprising a) introducing a RPE cell differentiated from an iPS cell by the method as defined into a subject, wherein said RPE cell comprises a bioluminescence label; b) detecting the bioluminescence signal of said RPE cell over time using an imaging method, thereby collecting imaging data; c) comparing the imaging data received in step b) to reference imaging data.

In a seventh aspect of the invention, the invention provides an in vitro method of determining the immunogenicity of a RPE cell differentiated from an iPS cell by the method as defined in a subject, to whom said differentiated RPE cell has been pre-delivered, the method comprising: a) detecting pro-inflammatory cytokine levels using an imaging method in a sample obtained from said subject, the sample comprising said differentiated RPE cell, thereby collecting imaging data; b) comparing the imaging data received in step a) to reference imaging data.

### BRIEF DESCRIPTION OF THE DRAWINGS

The invention will be better understood with reference to the detailed description when considered in conjunction with the non-limiting examples and the drawings, in which:

FIG. 1 shows a flow-diagram schematically representing the experimental steps of an illustrative embodiment of a method of generating an induced pluripotent stem cell of the present invention. The stem cells used herein are isolated from the amniotic membrane of the umbilical cord—also referred to as cord lining stem cells (CLSC). This embodiment starts with the harvesting of isolated CLSC by dissociating the cells from the cell culture device (it is however to be noted here that the CLSC can also be supplied for the method of the invention in isolated form). Then, the CLSC are counted and about 0.7 million cells are aliquoted into a microfuge and pelleted. The cells pellet is resuspended in a buffer suitable for electroporation before the plasmids encoding for the Yamanaka factors are added to the cells-buffer mixture. The electroporation is carried out with 1 pulse having a duration time of about 20 ms and a voltage of about 1600V or with 2 pulses having a duration time of 30 ms and a voltage of about 1350V for cord lining mesenchymal cells (CLMC) and cord lining epithelial cells (CLEC), respectively. After electroporation, the stem cells are immediately transferred into a medium suitable for recovery, wherein the medium contains a compound suppressing inflammatory response and enhancing cell survival. After a suitable time of recovery, the medium suitable for recovery is replaced with a 1:1 mixture of two different cell culture media, wherein the two different cell culture media are the medium suitable for recovery and a second cell culture medium. To refresh the cell culture medium, the media mixture is replaced with the same mixture of cell culture media about 4 days after electroporation. Thereby, colonies of cord lining induced pluripotent stem cells—also referred herein to as CLiPS—are generated. After about 2 further days, the 1:1 mixture of two different cell culture media is replaced with the second cell culture medium. This medium is also replaced about every second day to keep the medium fresh. When reaching a size of about 0.5 mm to 1.5 mm in diameter, the CLiPS colonies are picked and transferred to a coated cell culture vessel suitable for cell cultivation and proliferation. Again, the cell culture medium is replaced regularly with the same medium. After reaching a confluence of about 50%, the CLiPS colonies are detached from the coated culture device and transferred to another cell culture vessel suitable for cell cultivation and proliferation. This way, the CLiPS colonies are further dissociated. When reaching a confluence of about 70-80%, the CLiPS are passaged in a ratio of about 1:3 (v/v), wherein the passaging in a ratio of about 1:3 (v/v) is performed by contacting 1 volume dissociated CLiPS to 2 volume of fresh culture medium. The CLiPS are then cultivated in a medium containing a substance enhancing the survival of the cells until reaching a confluence of about 30-60%. At this point, the CLiPS are capable to be differentiated into any desired target cells.

FIG. 2 shows an exemplary comparison of the reprogramming efficiency of individual CLSC populations. The stem cells have been subjected to different electroporation settings to transfect the exogenous nucleic acid into the cells. The electroporation has been carried out using the electroporation parameters indicated in Okita et al, supra, (1650V, 10 ms, 3 pulses) and the respective parameters used in the present invention for transfection of epithelial stem cells of the amniotic membrane of the umbilical cord (also referred herein as “cord lining epithelial stem cell” or CLEC, 1350V, 30 ms, 2 pulses) and mesenchymal stem cells of the amniotic membrane of the umbilical cord (also referred herein to as cord lining mesenchymal stem cell or CLMC, (1600V, 20 ms, 1 pulse), respectively. 200K transfected cells were plated in triplicates in 6-well plates. About 21 days after transfection, the percentage reprogramming efficiency has been calculated as Colony number/200,000×10.

FIG. 3 shows exemplary colony development of induced pluripotent stem cells from human CLMC. FIG. 3a-f show a representative time course of colony development, wherein FIG. 3a depicts a typical morphology of human CLMC cultured in its maintenance medium at Day 0 of cultivation. FIG. 3b depicts a typical morphology of human CLMC cultured in its maintenance medium at Day 15 of cultivation. FIG. 3c depicts a typical morphology of human CLMC cultured in its maintenance medium at Day 24 of cultivation. FIG. 3d depicts a typical morphology of human CLMC cultured in its maintenance medium at Day 29 of cultivation. FIG. 3e shows a 4× magnification of the typical morphology of an iPS colony first passage and 3f shows a 10× magnification of the typical morphology of an iPS colony at first passage. FIG. 3g-l depict an exemplary immunofluorescence staining of iPS derived from human cord lining cells showing the activation of endogenous expression of pluripotent embryonic stem cell markers, wherein FIG. 3g shows the expression of KLF4, FIG. 3h shows the expression of NANOG, FIG. 3i shows the expression of OCT3/4, FIG. 3j shows the expression of SOX2, FIG. 3k shows the expression of SSEA4 and FIG. 3l shows the expression of Tra-1-60. FIG. 3m shows an exemplary karyotype analysis demonstrating normal chromosomal numbers and G-banding patterns of CLiPS in the individual cell lines CLEC23 (EC23-CLiPS), CLMC23 (MC23-CLiPS), CLEC44 (EC44-CLiPS) and CLMC44 (MC44-CLiPS). FIG. 3n shows an exemplary human CLMSC-DTHN culture emerging 10 days of reprogramming magnified 20× FIG. 3o shows exemplary a morphology of expanding human CLMSC-DTHN cultured on laminin-511 substrate magnified 4×. FIG. 3p shows exemplary a morphology of expanding human CLMSC-DTHN cultured on laminin-511 substrate magnified 10×. FIG. 3q shows exemplary a morphology of expanding human CLMSC-DTHN cultured on laminin-511 substrate magnified 20×. FIG. 3r shows an exemplary expression of the human pluripotent marker NANOG in CLMSC-DTHN iPS at passage No. 3. FIG. 3s shows an exemplary expression of the human pluripotent marker OCT3/4 in CLMSC-DTHN iPS at passage No. 3. FIG. 3t shows an exemplary expression of the human pluripotent marker SOX2 in CLMSC-DTHN iPS at passage No. 3. FIG. 3u shows an exemplary expression of the human pluripotent marker NTRA-1-81 in CLMSC-DTHN iPS at passage No. 3. Scale bars: all 100 μm. FIG. 3v shows an exemplary RT-PCR analyses of reprogramming gene expression and pluripotent gene expression in primary parental cells, parental cells 11 days after vector transfection (D11 transfected cells) and in established iPS clones (CLiPS). ‘Vec’ denotes amplification specific for vector derived sequences. Glycerinaldehyd-3-phosphat-Dehydrogenase (GAPDH) was used as an internal control. PCR of Homo sapiens (H1) total RNA without reverse transcription was used to control for genomic contamination for all primer pairs.

FIG. 4 shows an exemplary histological analysis of a teratoma formed by immunocompromised non-obese diabetic severe combined immunodeficiency (NOD-SCID) mice after CLiPS injection. The teratoma formation assay reveals the formation of all three germ layers. FIG. 4a-inset shows a teratoma obtained from human CLEC-derived iPS 3 months after subcutaneous injection. Sections of the teratoma are further analyzed by a hematoxylin and eosin staining. FIG. 4a shows the presence of respiratory-like epithelium in the teratoma. FIG. 4b shows the presence of glandular structures representing the endoderm in the teratoma. In FIG. 4c the arrowhead shows the presence of cartilage in the teratoma. In FIG. 4d the arrowhead shows the presence of bone representing the mesoderm in the teratoma. FIG. 4e shows the presence of renal tissue in the teratoma. The filled arrowheads indicate glomeruli and the hollow arrowheads indicate renal tubules.

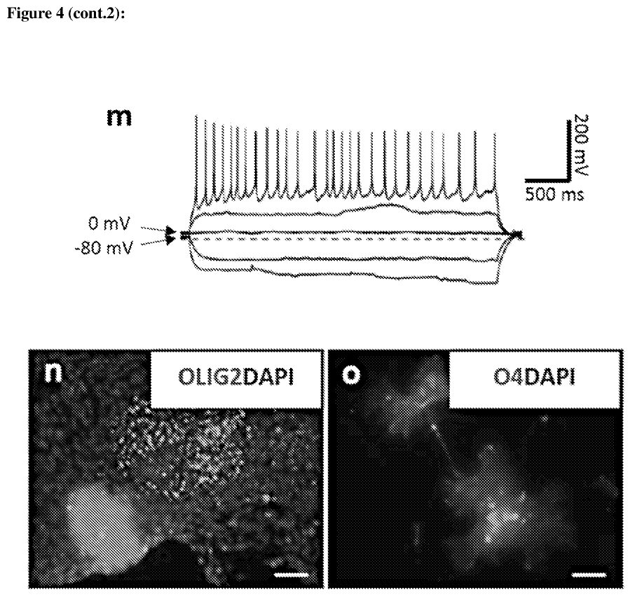

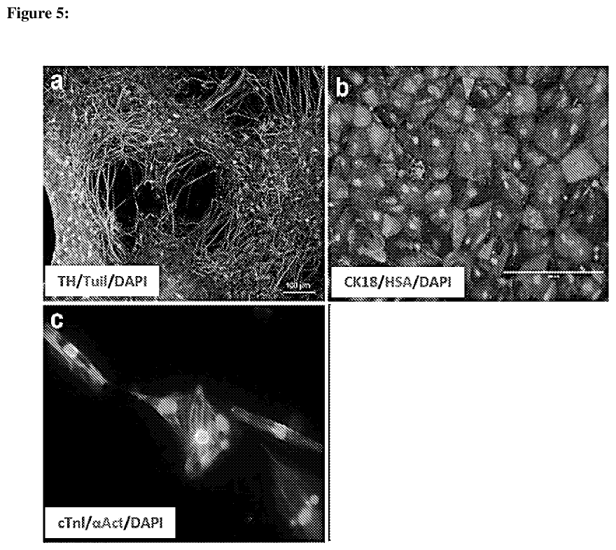

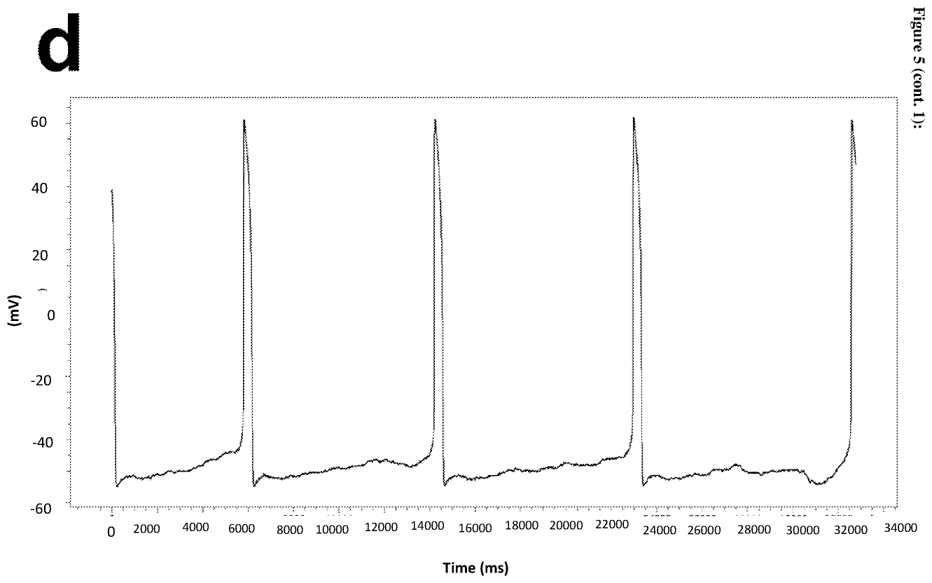

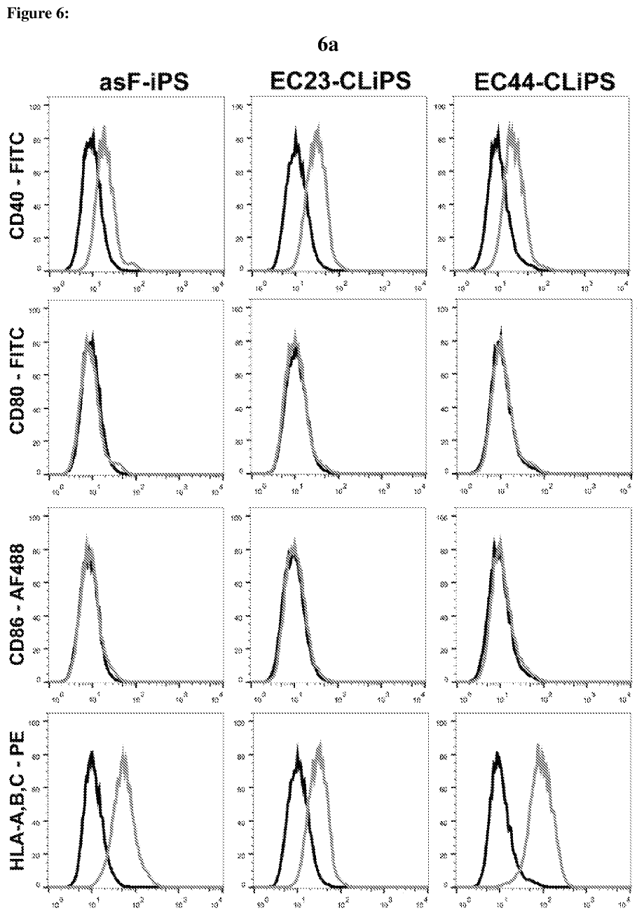

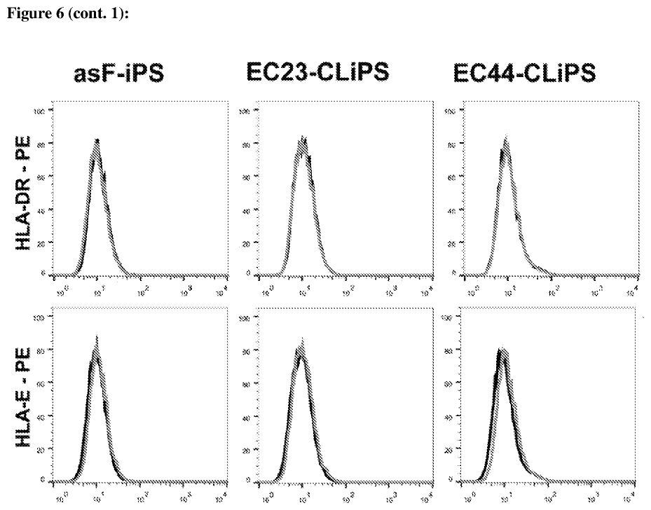

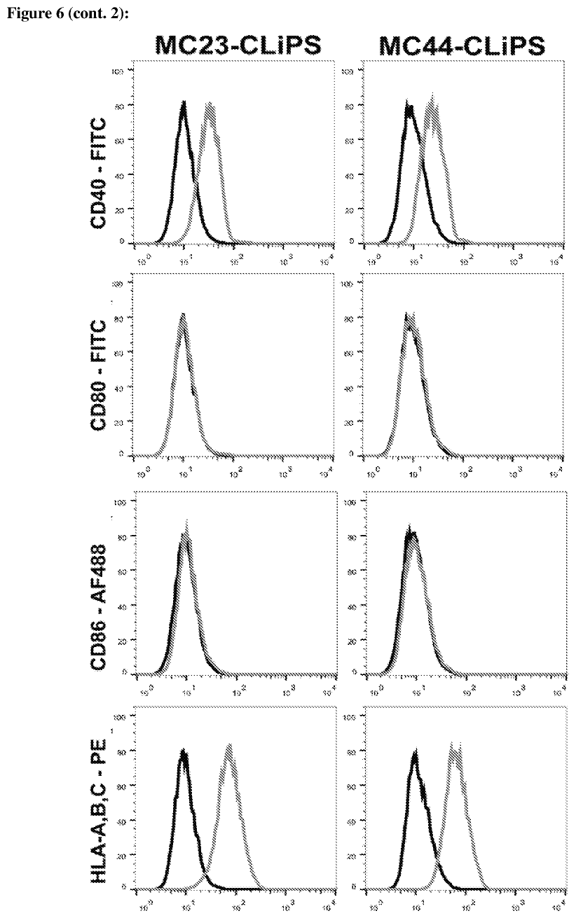

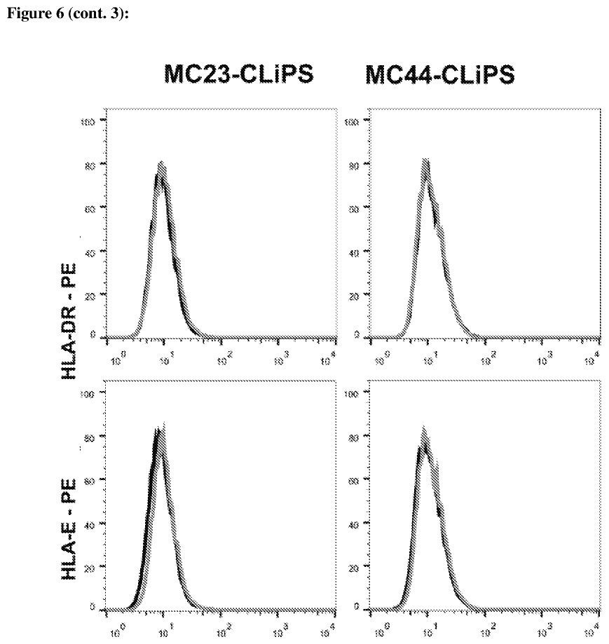

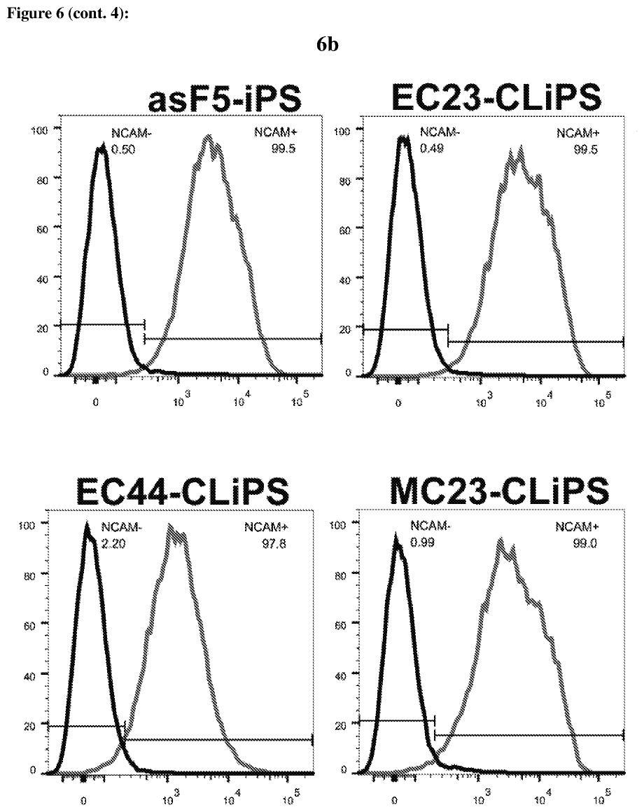

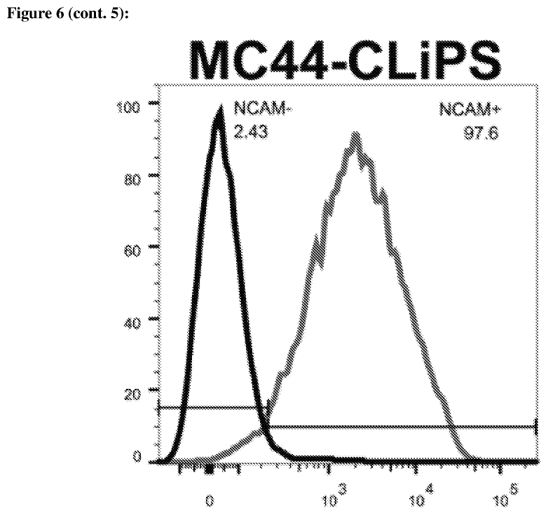

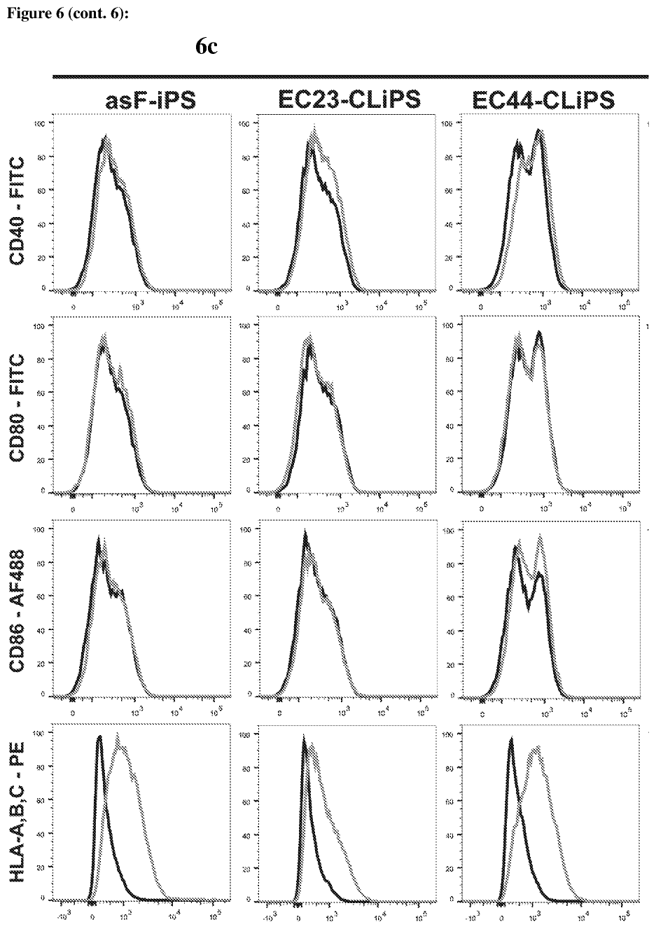

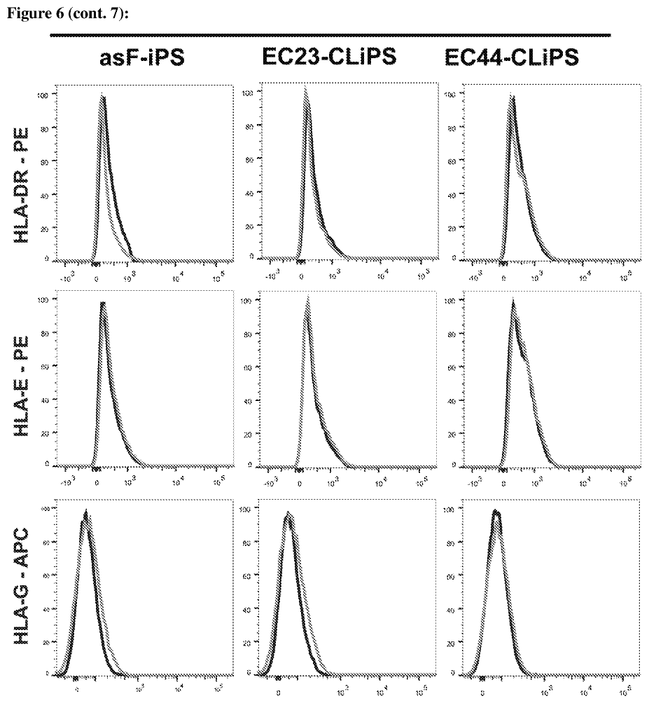

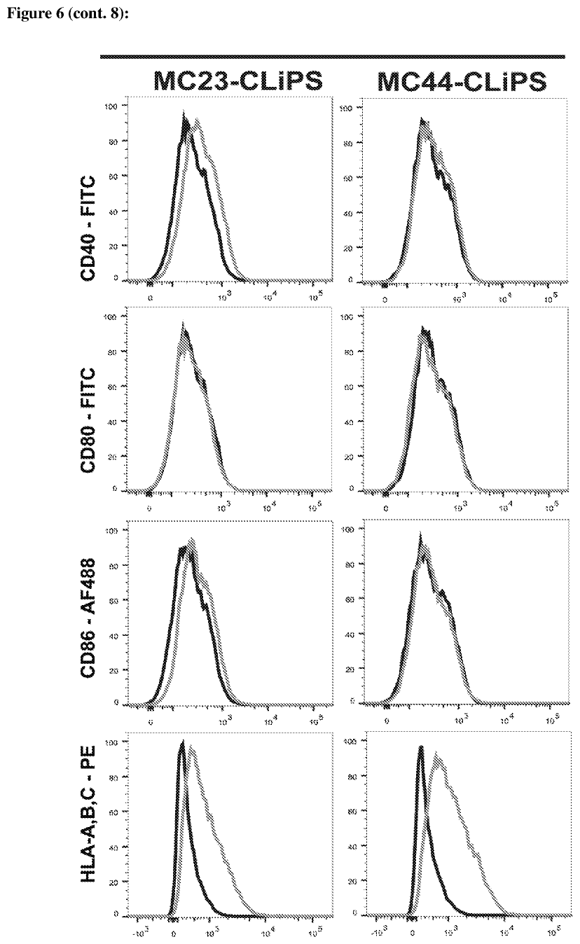

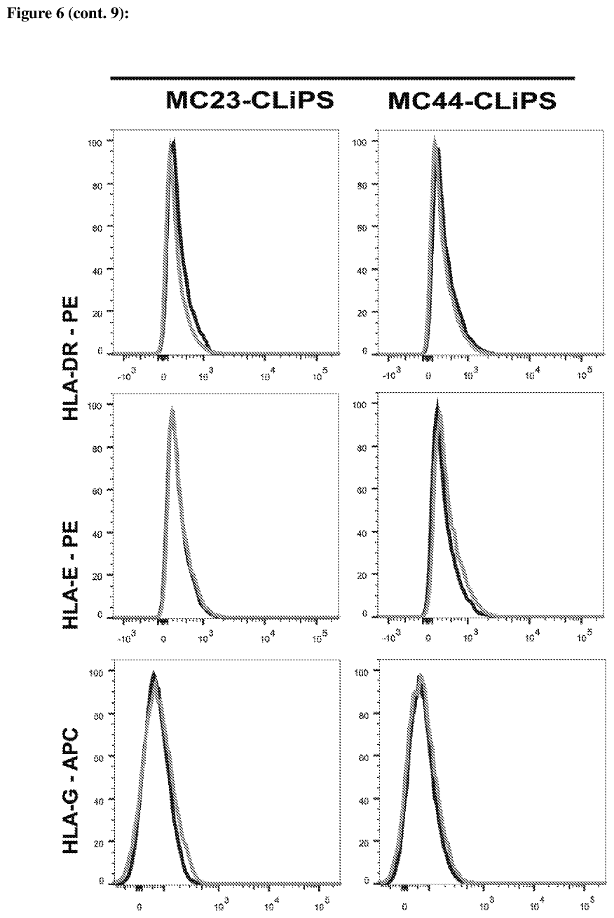

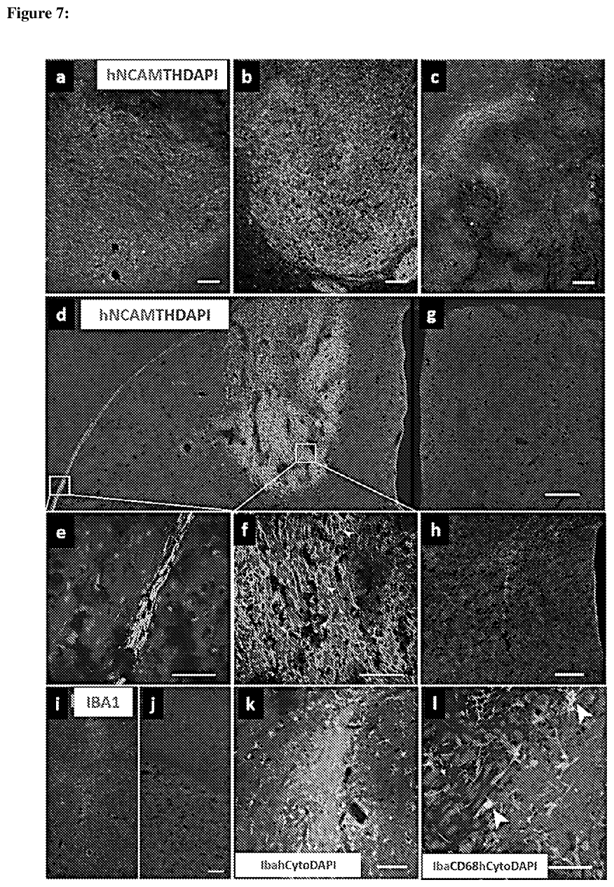

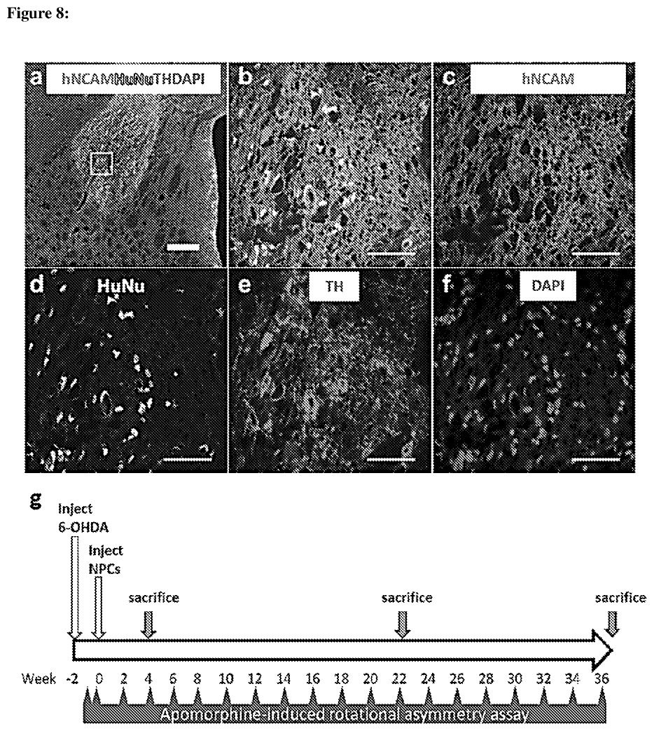

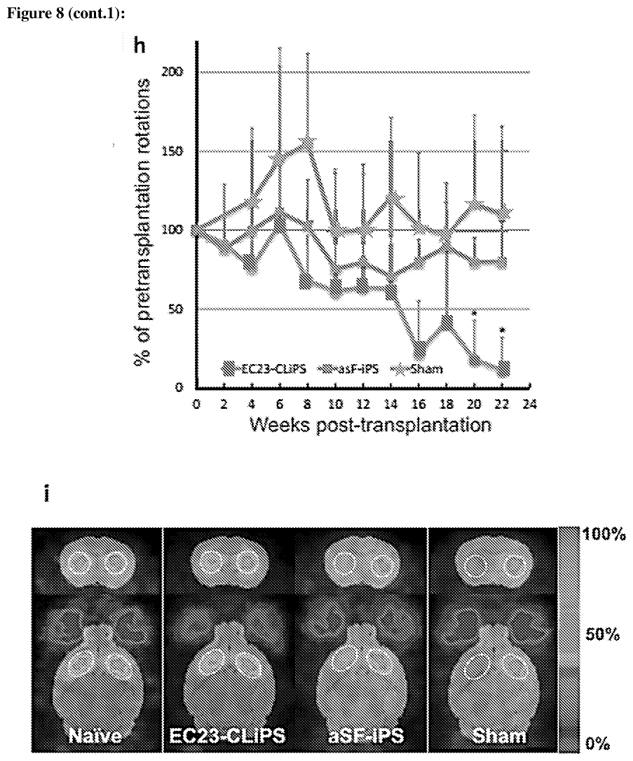

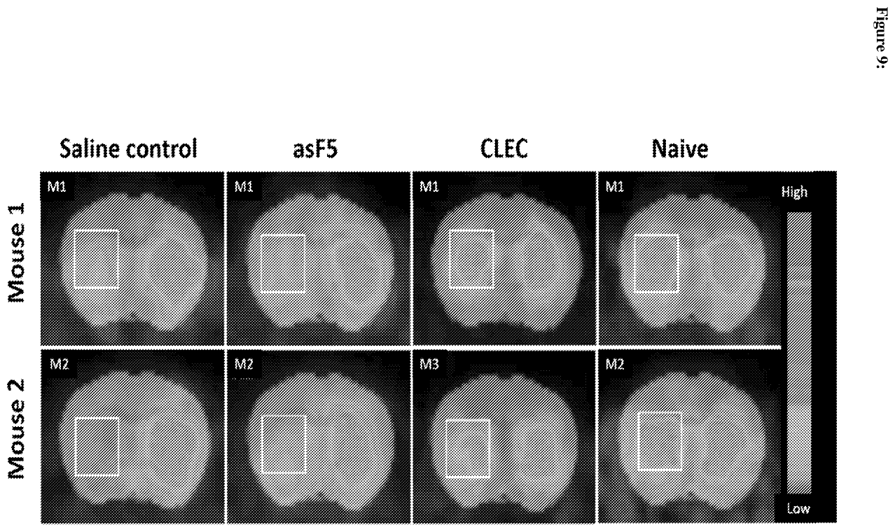

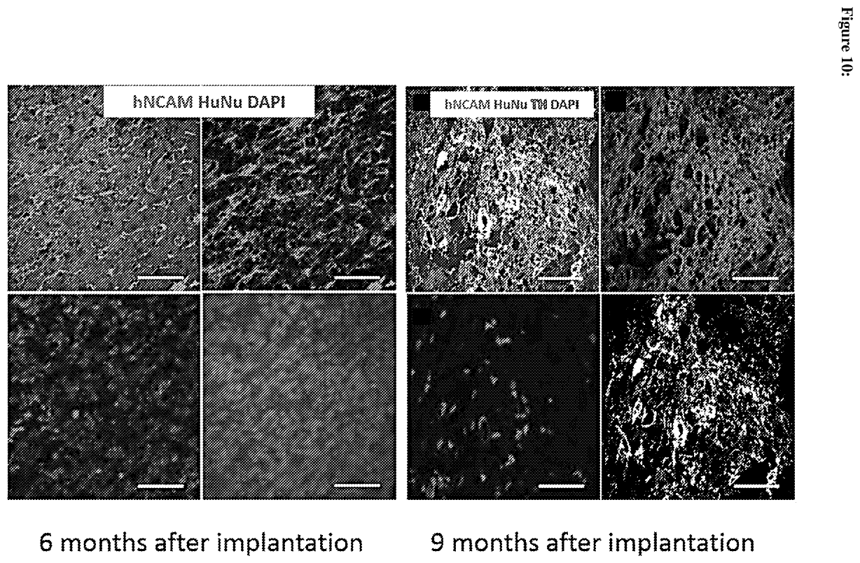

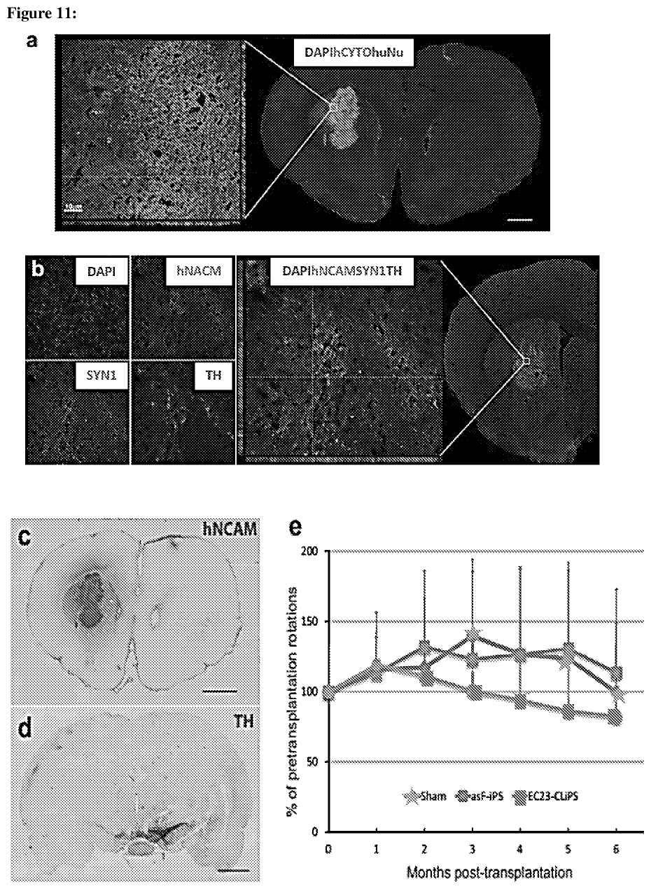

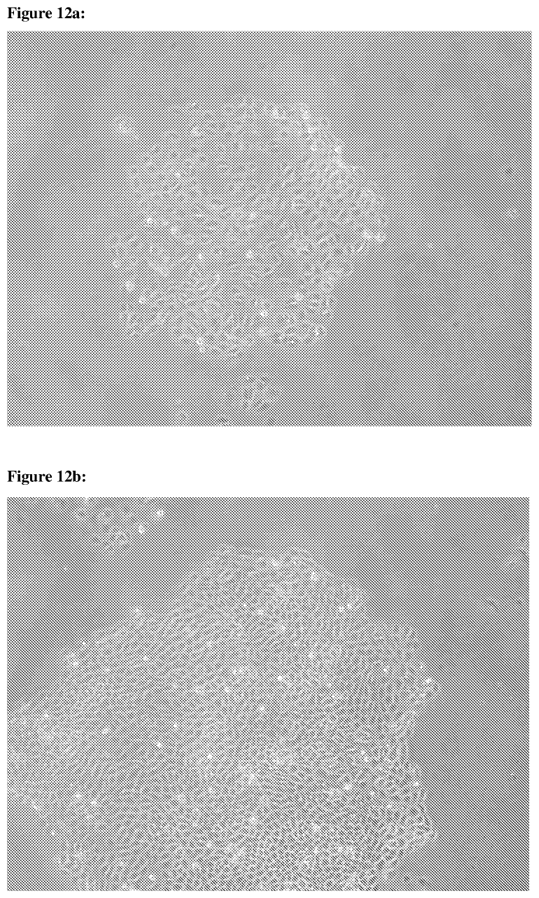

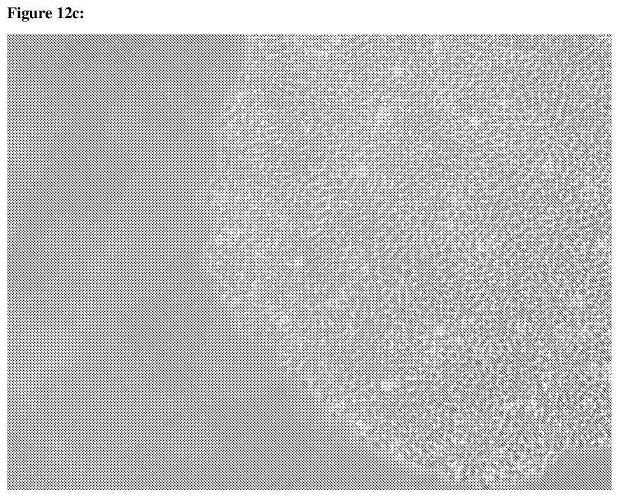

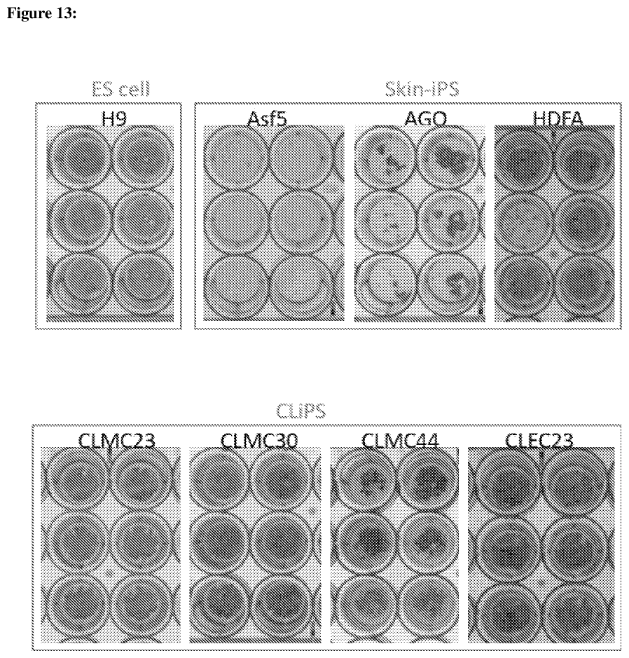

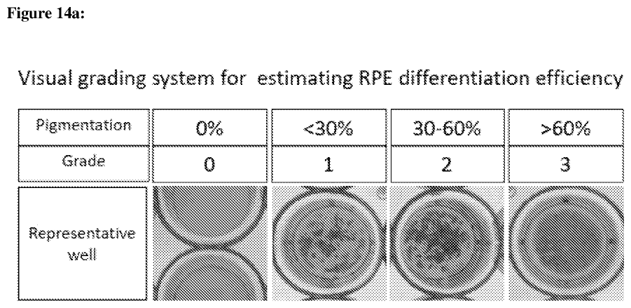

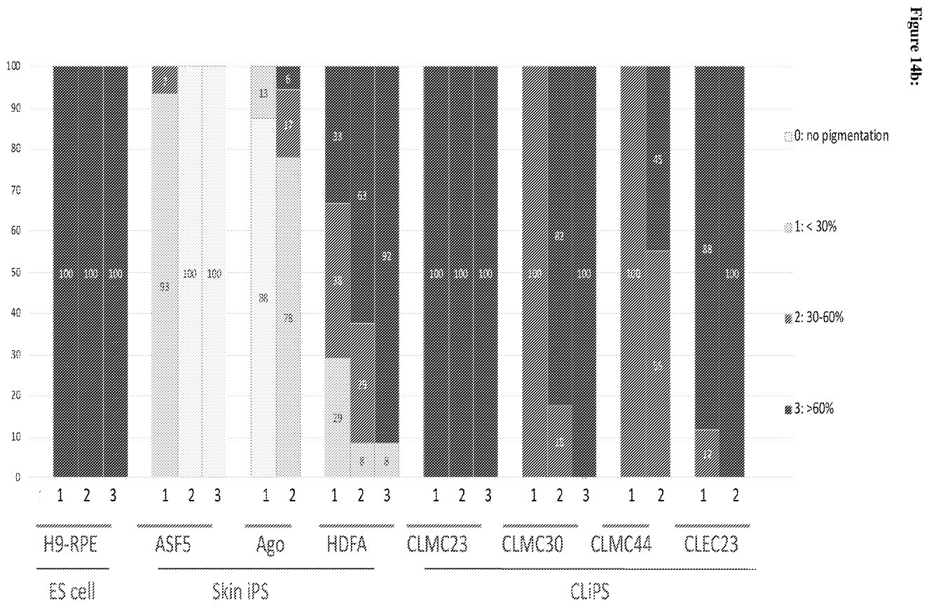

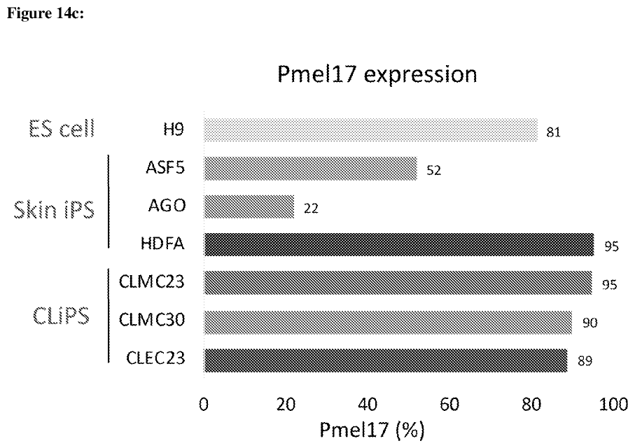

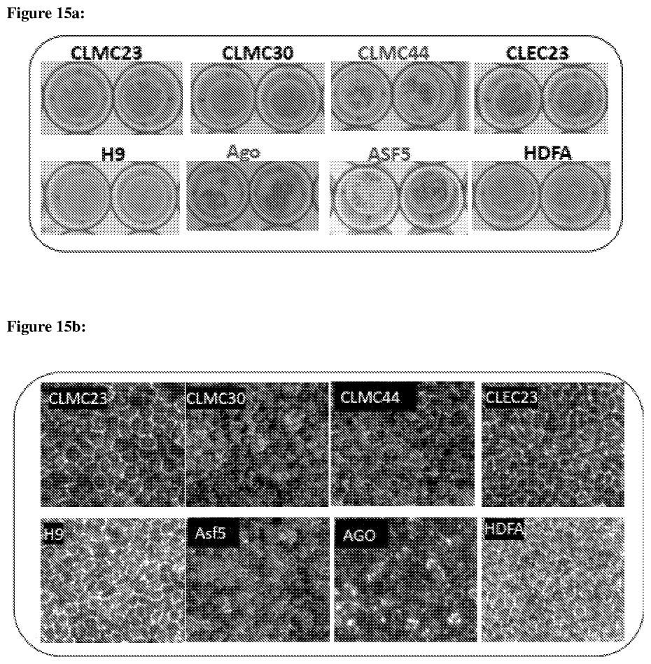

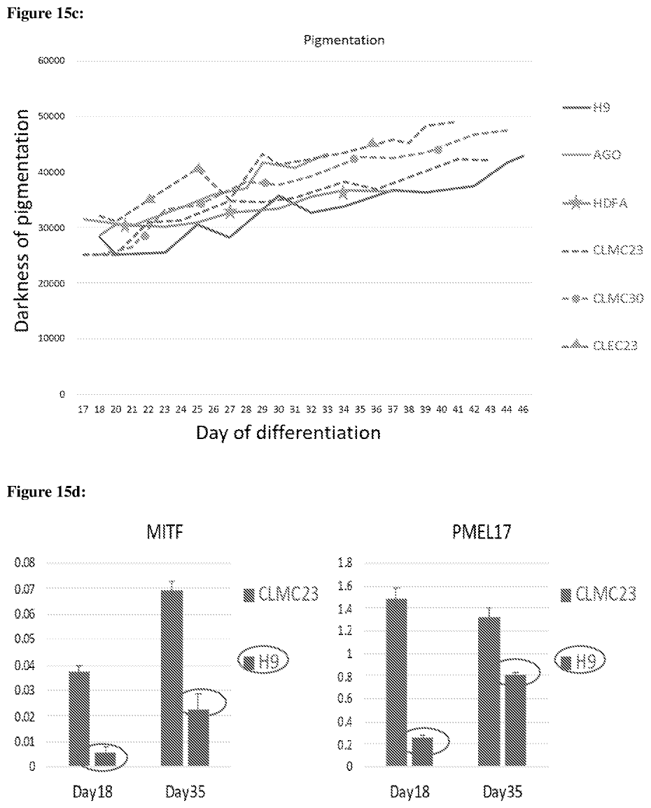

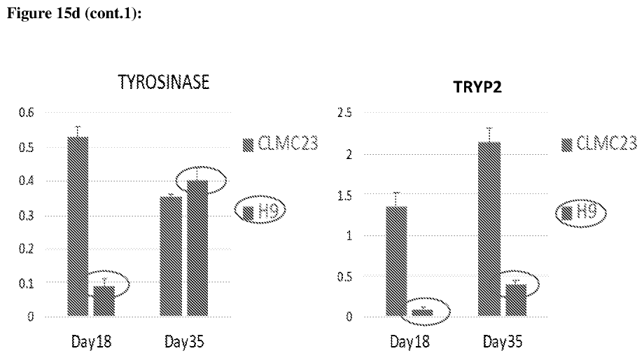

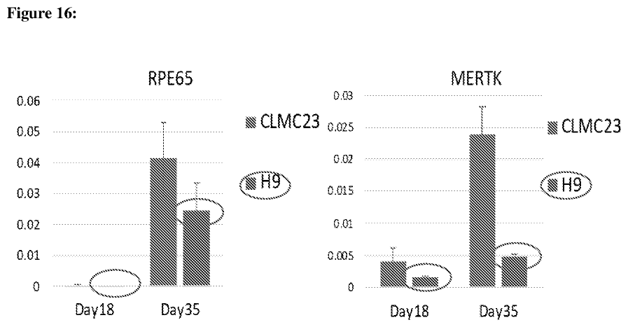

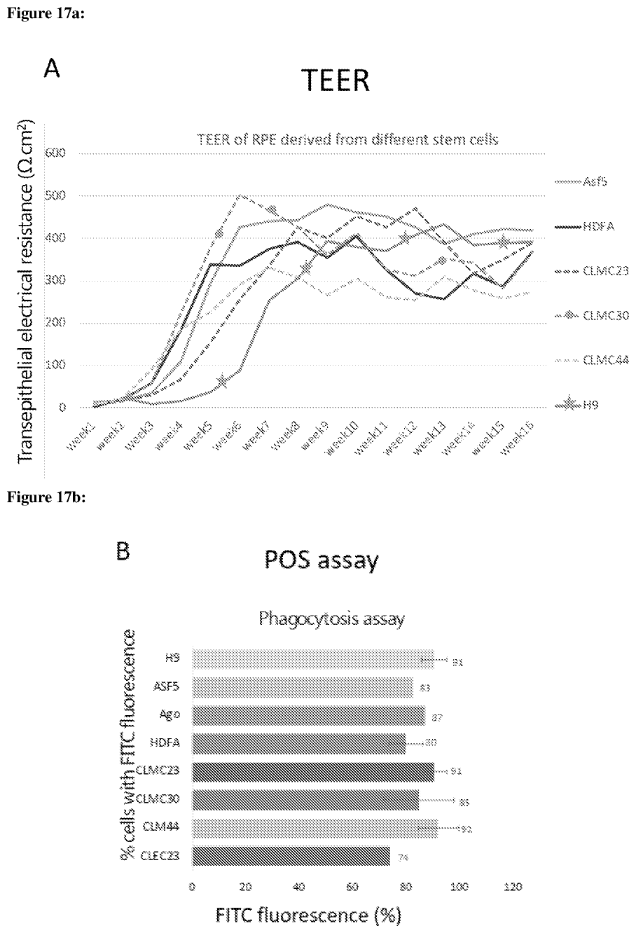

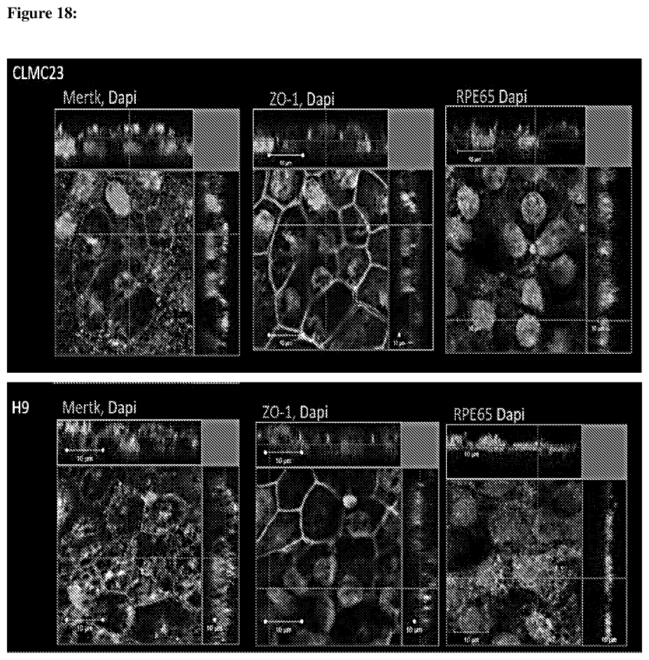

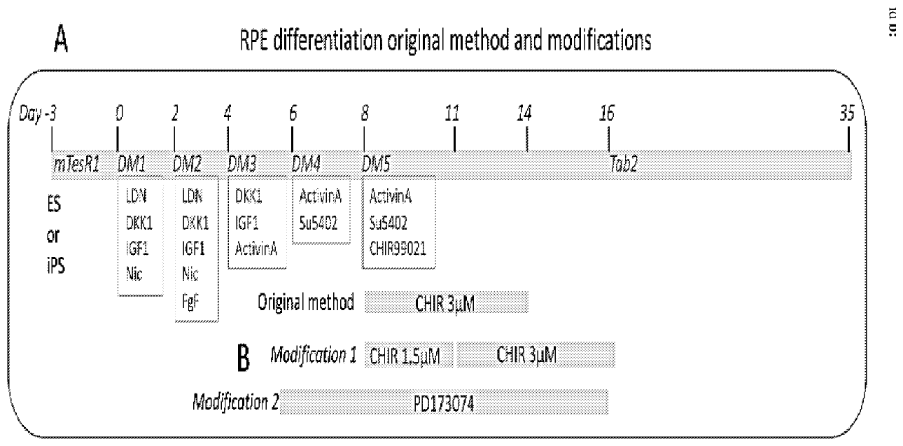

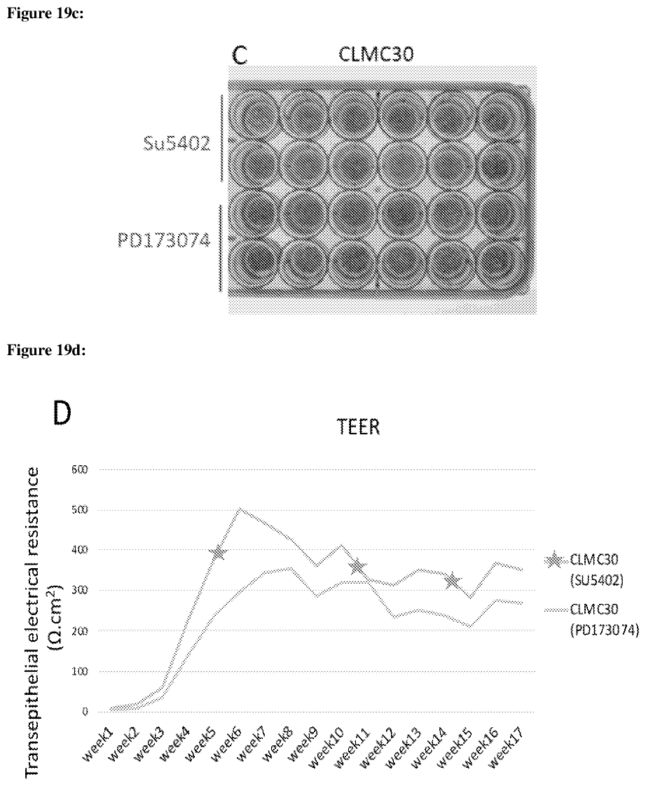

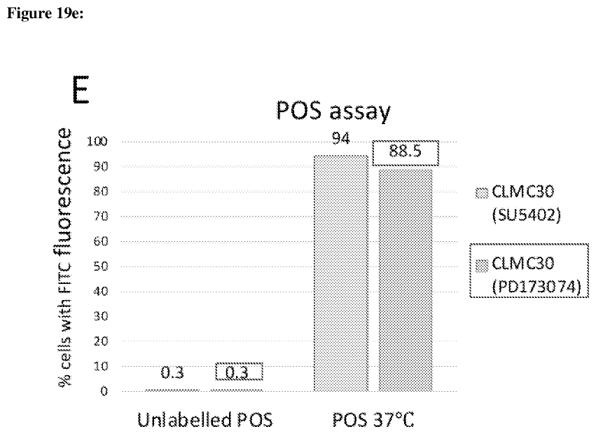

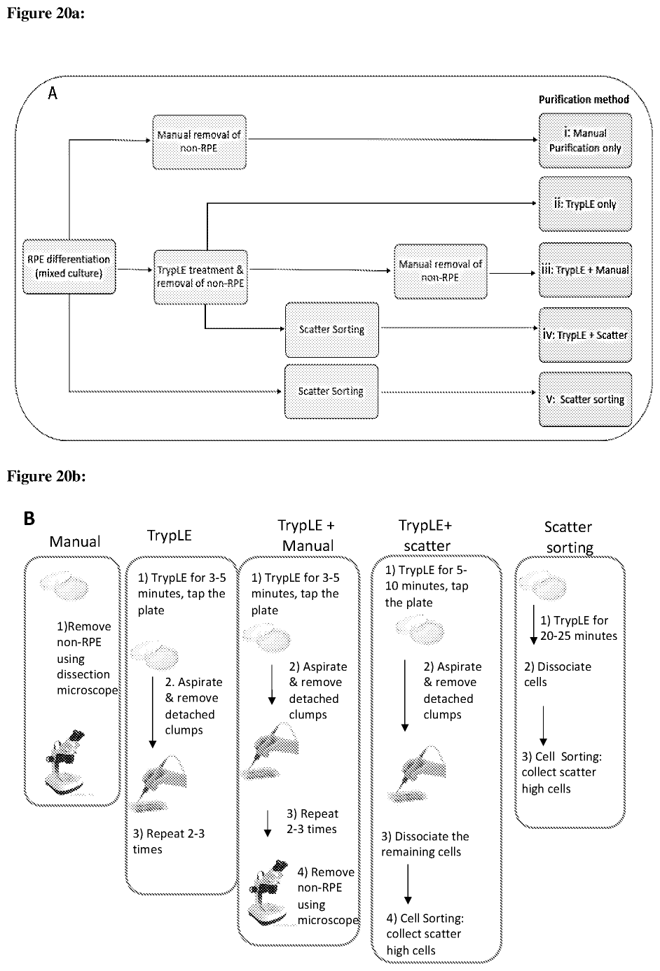

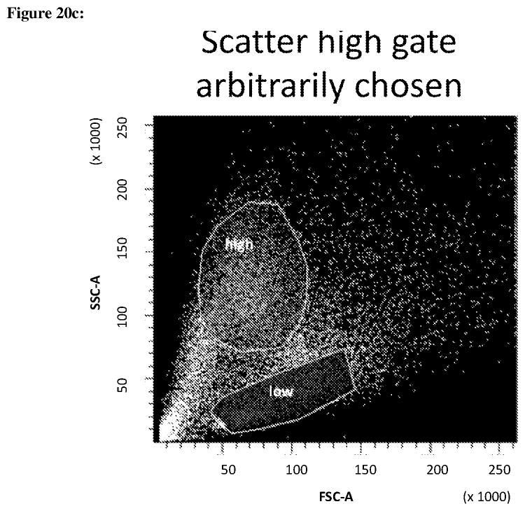

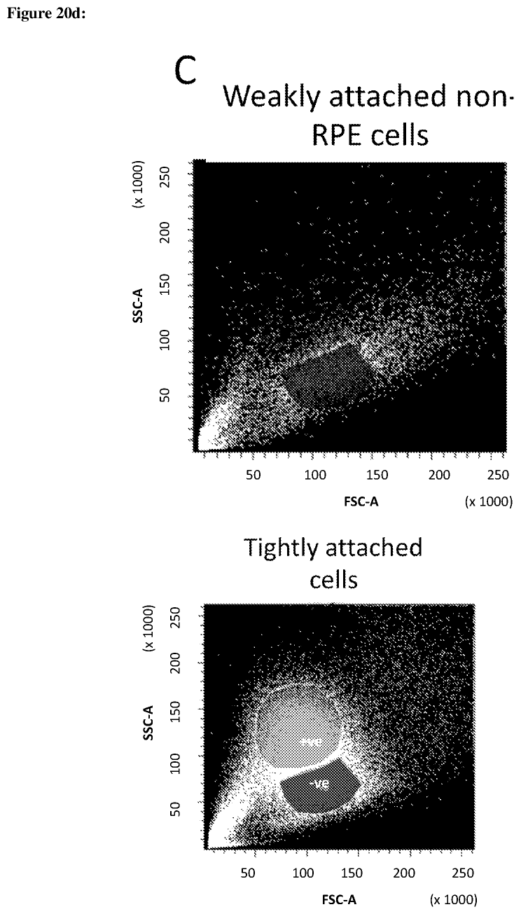

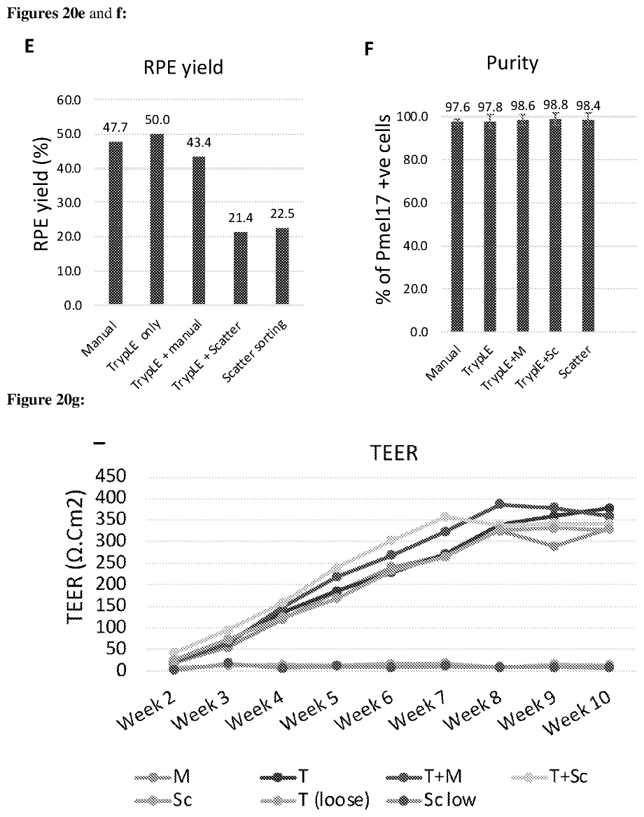

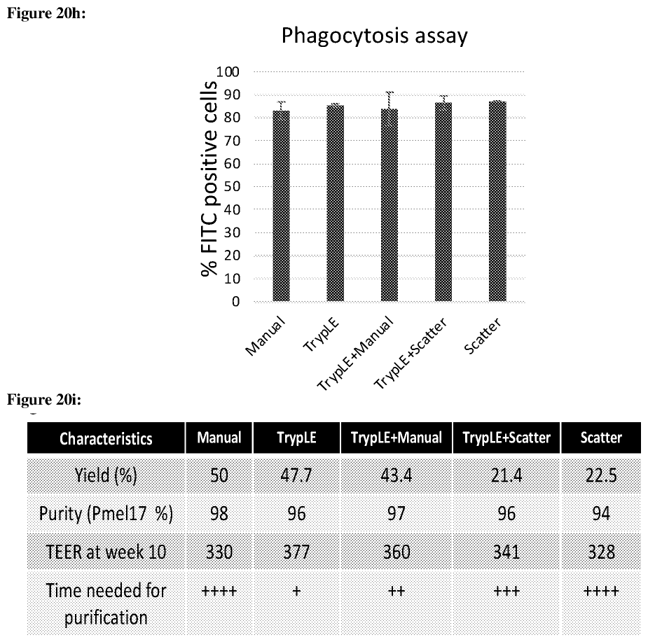

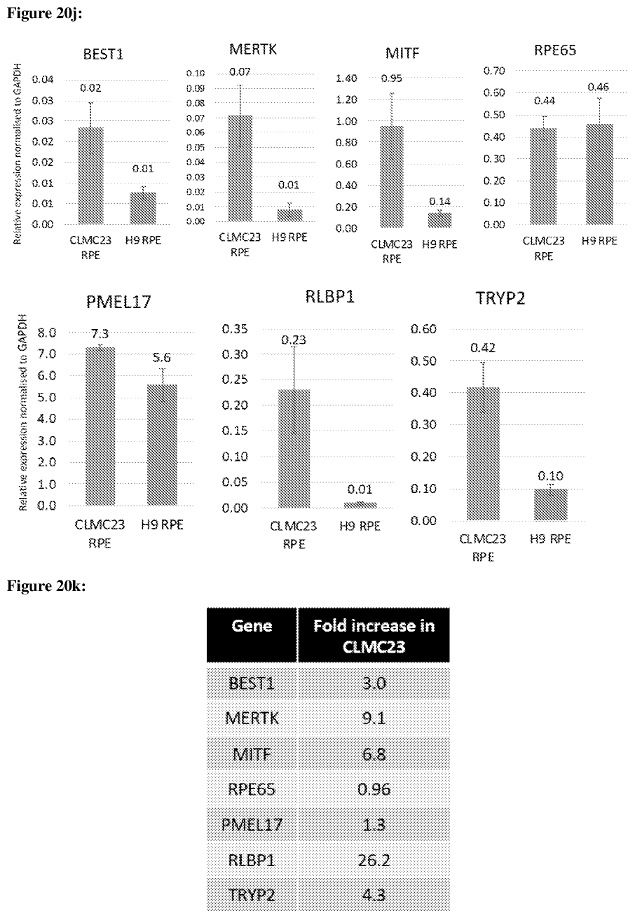

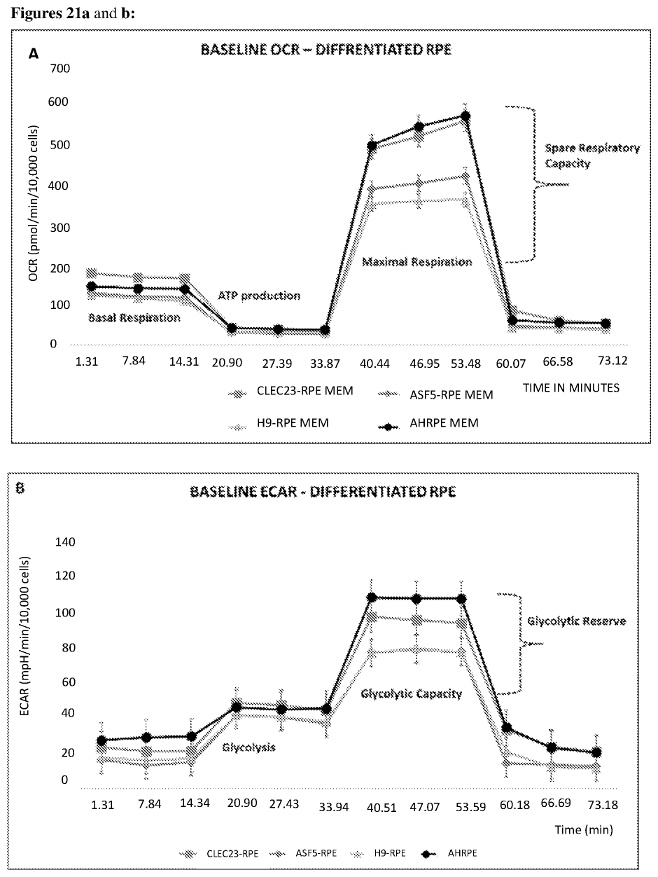

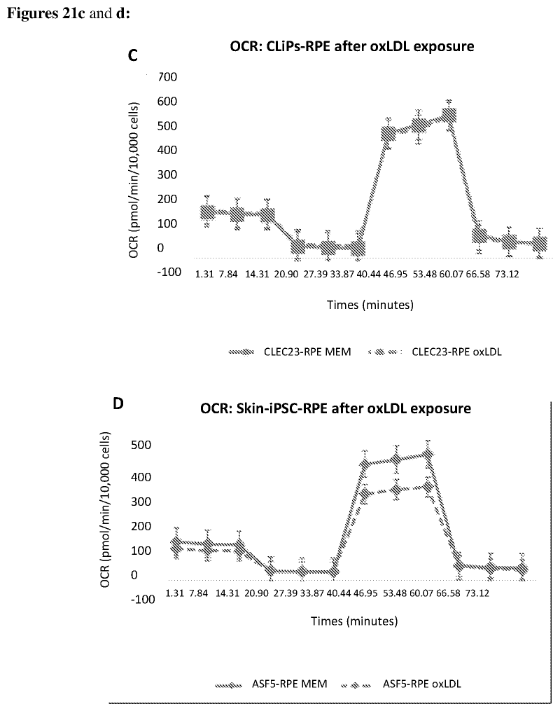

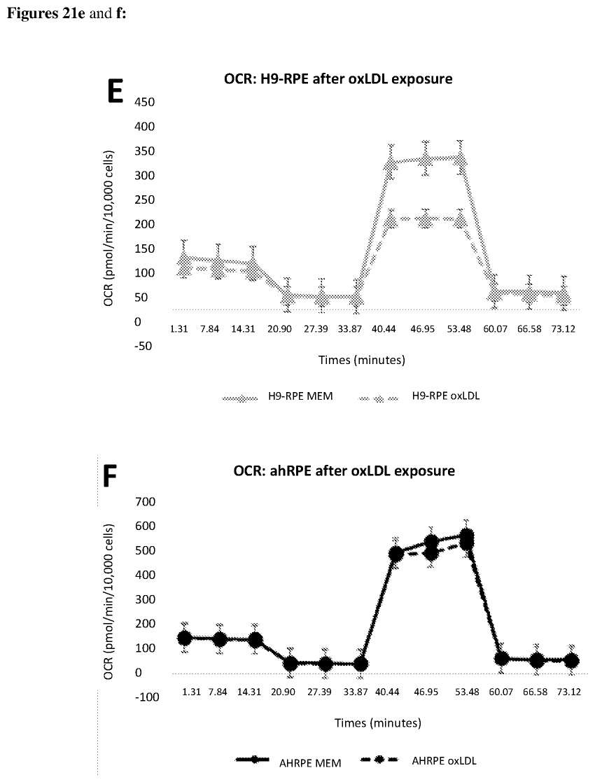

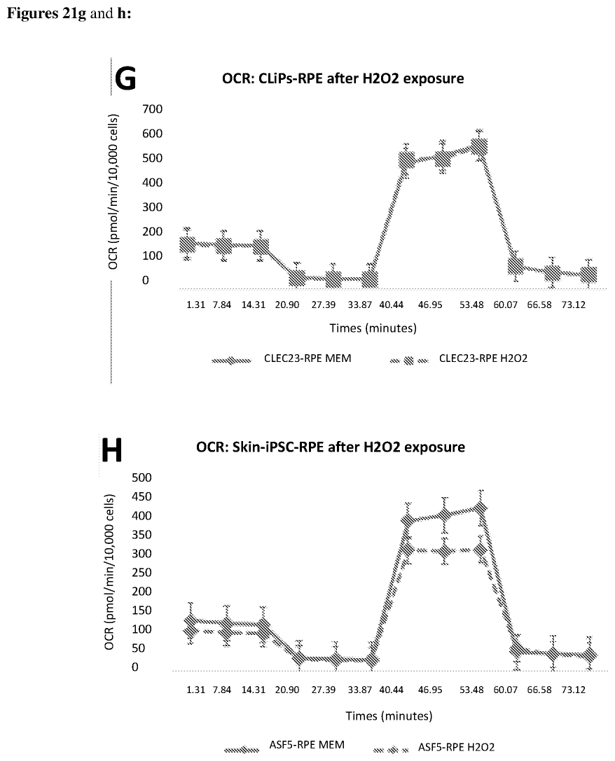

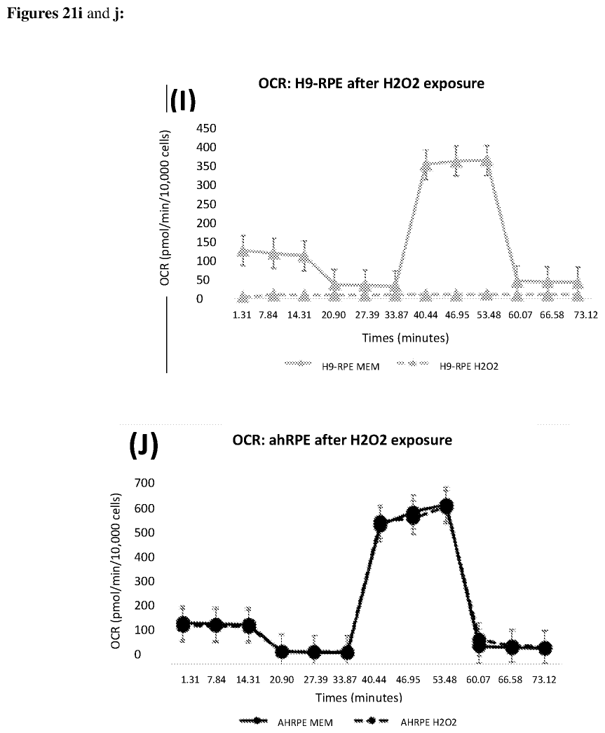

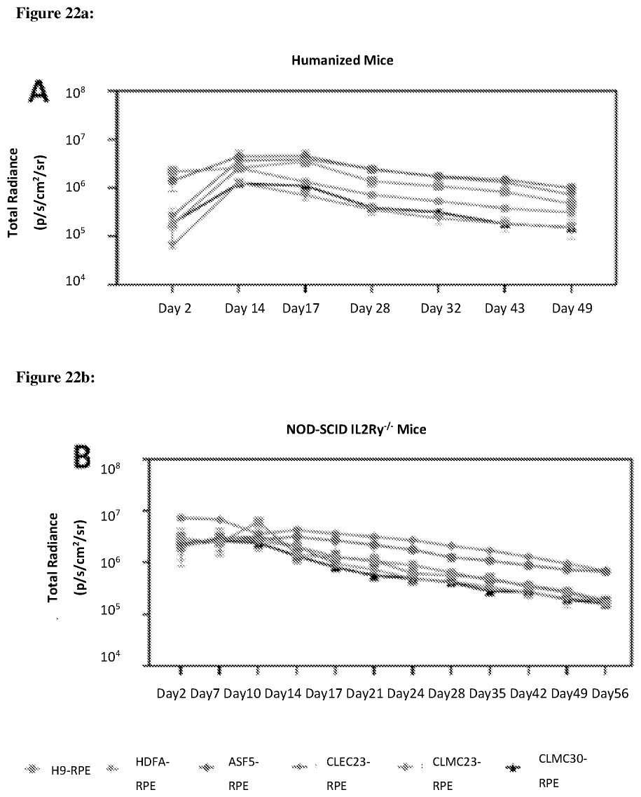

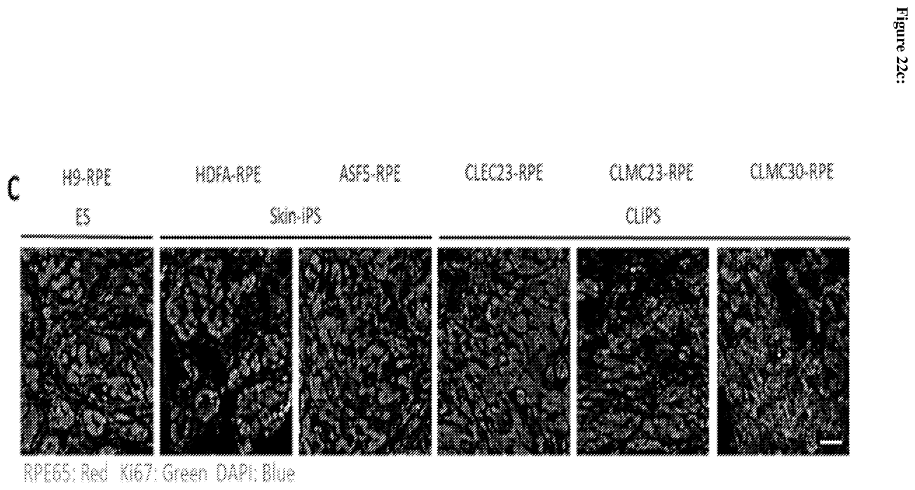

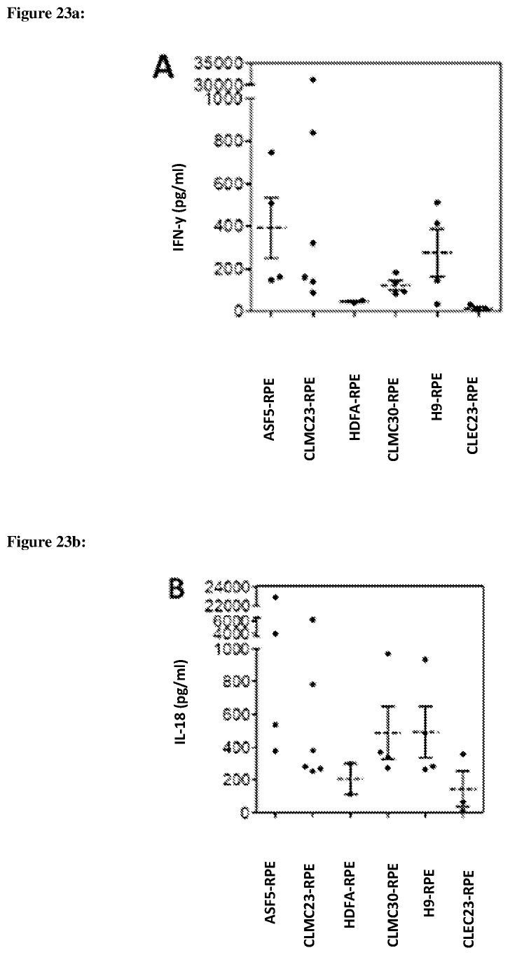

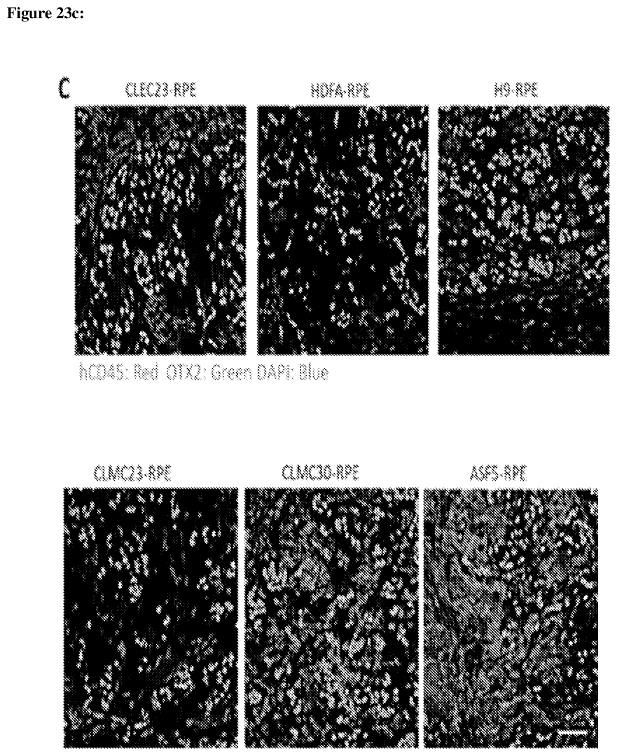

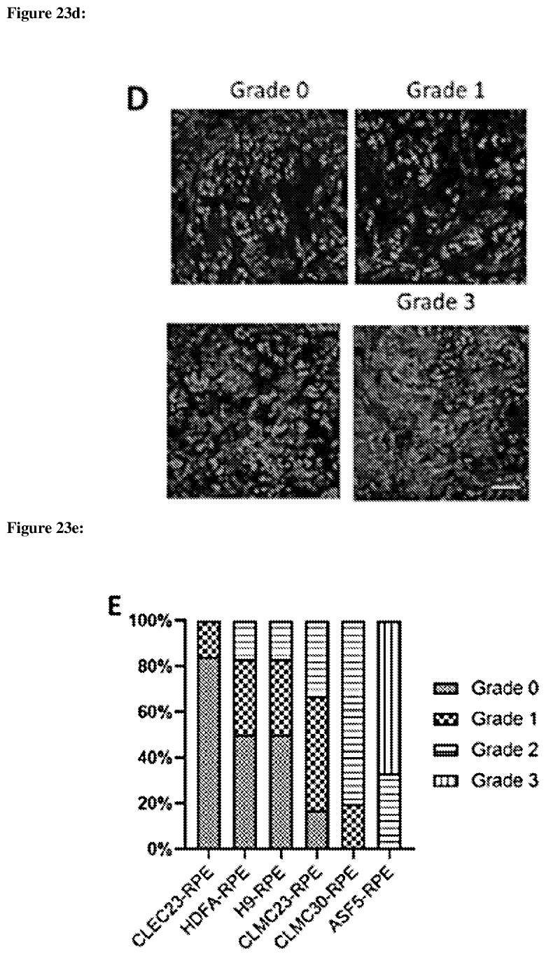

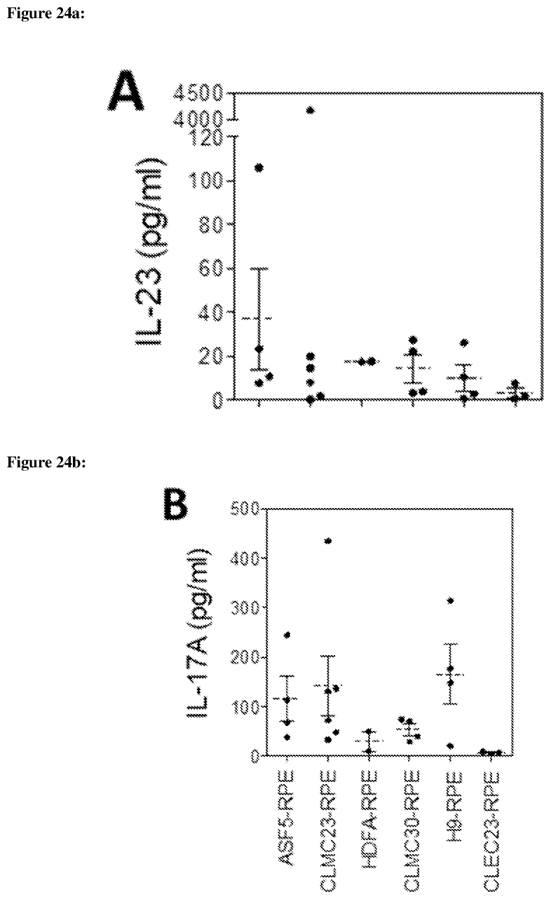

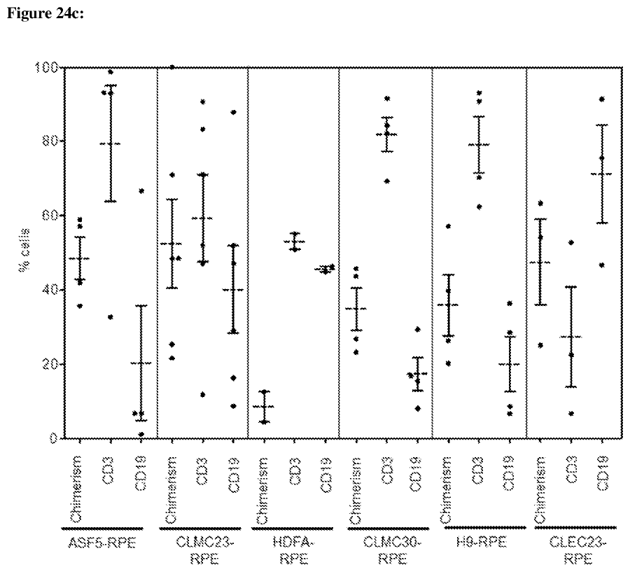

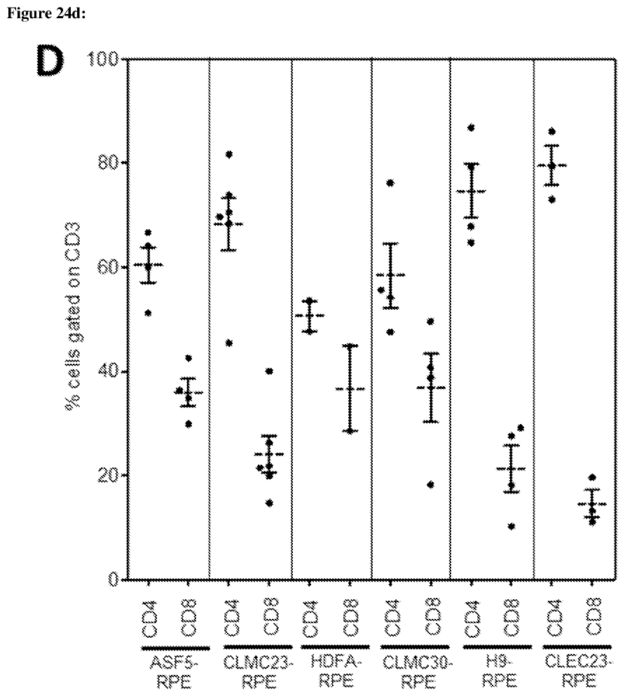

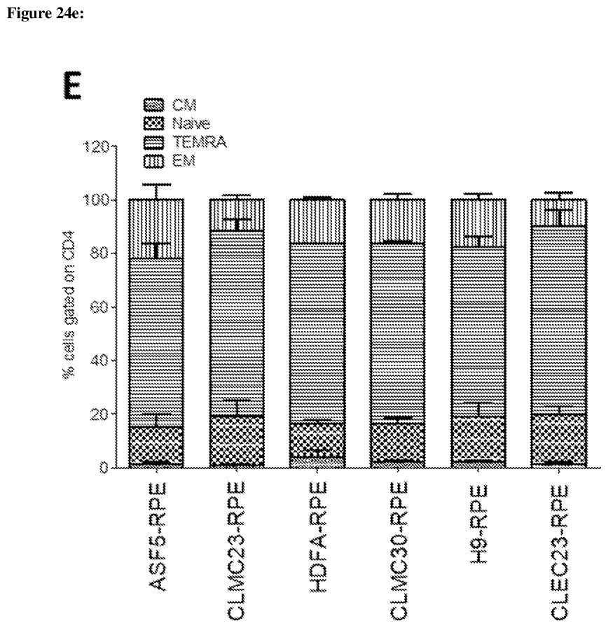

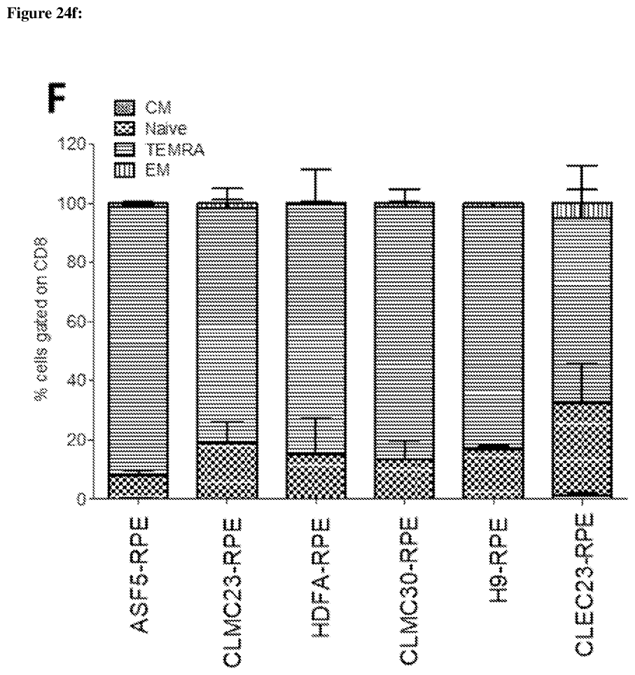

In FIG. 4f the arrowhead shows the presence of neural epithelium representing the ectoderm in the teratoma. Using directed differentiation protocols, CLiPS were induced to differentiate into specific tissues. FIG. 4g shows CLiPS differentiated into hepatocytes visualized with alpha-fetoprotein (AFP) and 4′,6-diamidino-2-phenylindole (DAPI). FIG. 4h shows CLiPS differentiated into hepatocytes visualized with human serum albumin (HAS), cytokeratin 18 (CK18) and DAPI. FIG. 4i shows CLiPS differentiated into hepatocytes visualized with Oil Red O. FIG. 4j shows CLiPS differentiated into cardiomyocytes visualized with alpha-actinin (αACT), cardiac troponin I (cTnl), myosin regulatory light chain 2a (MLC2a) and DAPI. FIG. 4k shows CLiPS differentiated into dopaminergic neurons visualized with the floor-plate marker FOXA2, the roof plate marker LMX1A and DAPI. FIG. 4l shows CLiPS differentiated into dopaminergic neurons visualized with neuron-specific class III beta-tubulin (TUJI) and tyrosine Hydroxylase (TH). FIG. 4m shows CLiPS differentiated into oligodendrocyte progenitor cells visualized with OLIG2 and DAPI. FIG. 4n shows CLiPS differentiated into oligodendrocyte progenitor cells visualized with 04 and DAPI. FIG. 4o shows an electrophysiological analysis of mature human CLiPS-derived dopaminergic neurons at Day 45 of differentiation. The human CLiPS-derived dopaminergic neurons fire trains of action potential with injected currents. Scale bars: 200 μm in FIG. 4a, FIG. 4c and FIG. 4d; 100 μm in FIG. 4b, FIG. 4e and FIG. 4f; 50 μm in FIG. 4g, FIG. 4h, FIG. 4i, FIG. 4k, FIG. 4l, FIG. 4m; 25 μm in FIG. 4j, FIG. 4n.

FIG. 5 shows an exemplary directed differentiation of human CLiPS into various different cell types, wherein FIG. 5a depicts human CLiPS-derived neurons visualized with TH, Tuik and DAPI, FIG. 5b depicts human CLiPS-derived hepatocytes visualized with CK18, HAS and DAPI, FIG. 5c depicts human CLiPS-derived cardiomyocytes visualized with cTnl, αAct and DAPI and FIG. 5d shows an electrophysiological analysis of contracting human CLiPS-derived cardiomyocytes illustrating the cells generating spontaneous action potentials.

FIG. 6 shows an exemplary flow cytometric analysis of major histocompatibility complex (MHC) Class I and II, and T-cell co-stimulatory protein expression on iPS and dopaminergic neuroprogenitors differentiated from them. FIG. 6a shows a flow cytometric profile of immune-related gene expression on undifferentiated iPS. FIG. 6b shows a flow cytometric analysis of neural cell adhesion molecule (NCAM)-positive populations. These populations were gated for an analysis of immune-related protein expression. FIG. 6c shows an analysis of immune-related protein expression on Day25 differentiated dopaminergic neuroprogenitors.

FIG. 7 shows an in vivo comparison of engraftment of dopaminergic neuronal progenitor cells (NPCs) derived from human CLiPS and human adult fibroblast-iPS (asF-iPS) in NOD-SCID mice. The day 25 dopaminergic NPCs were injected into the striatum of NOD-SCID mice to assess the engraftment and differentiation potential of the cells in an immune-deficient environment. TH-immunoreactive dopaminergic neurons are present among abundant human NCAM-positive engrafted neurons. FIG. 7a shows in vivo engraftment of day 25 dopaminergic NPCs derived from human asF-iPS. FIG. 7b shows in vivo engraftment of day 25 dopaminergic NPCs derived from human CLEC-iPS (EC23-CLiPS). FIG. 7c shows in vivo engraftment of day 25 dopaminergic NPCs derived from CLMC-iPS (MC23-CLiPS). FIG. 7d shows an antibody staining of the grafted hemisphere of a Parkinson's Disease (PD) mouse model created in an immunocompetent C57BL/6NTac mouse 1 month after transplantation with human CLEC-iPS-derived dopaminergic NPCs. Human NCAM (green) and TH (red) double positive neurons are present in abundance in the injected site. FIG. 7e shows long neuronal processes originating from the graft site projected along the forceps major of the corpus callosum to distal regions of the brain. Arrowheads in FIG. 7f indicate the human NCAM and TH double positive neurons, which are present in abundance in the injected site, as shown by the arrowheads. FIG. 7g shows the contralateral non-transplanted hemisphere of the same section as shown in FIG. 7d. FIG. 7h illustrates that no surviving cells are visible in striatum transplanted with human adult asF-iPS-derived NPCs suggesting immune rejection. FIG. 7i indicates abundant microglia/macrophage aggregation in the transplanted hemisphere. FIG. 7j shows absence of microglia/macrophage aggregation in the non-transplanted hemisphere. FIG. 7k shows a higher magnification of FIG. 7i It can be seen that microglia located proximal to and inside graft display a more amoeboid morphology characteristic of activated microglia. FIG. 7l shows a higher magnification of FIG. 7k indicating an expression of CD68, which is an activation marker for microglia. Scale bars: 100 μm in FIG. 7a-c and FIG. 7k; 200 μm in FIG. 7d, FIG. 7g and FIG. 7h; 50 μm in FIG. 7e, FIG. 7f and FIG. 7l.

FIG. 8 shows the survival of human CLEC derived (EC23-CLiPS) dopaminergic neurons in mouse PD model 9 months after transplantation. FIG. 8a indicates HuNu+/hNCAM+/TH+ neurons present in the transplanted hemisphere. FIG. 8b is an overlay of FIGS. 8c-f and shows a higher magnification of the boxed area in FIG. 8a. FIG. 8c indicates hNCAM+ neurons present in the transplanted hemisphere. FIG. 8d indicates HuNu+ neurons present in the transplanted hemisphere. FIG. 8e indicates TH+ neurons present in the transplanted hemisphere. FIG. 8f indicates nuclei of the neurons present in the transplanted hemisphere. FIG. 8g illustrates schematically the experimental steps starting from the induction of PD lesion by 6-hydroxydopamine (6-OHDA) injection into the striatum of C57BL/6NTac mice. Pre-transplantation rotation behavioral assays were performed one and two weeks prior to NPC transplantation. FIG. 8h shows the results of an Apomorphine-induced rotational asymmetry assay in mice transplanted with dopaminergic NPCs derived from human EC23-CLiPS and asF-iPS, and sham control. The assays were performed every two weeks up to 22 weeks after transplantation. Animals in the human EC23-CLiPS group showed statistically significant rotational recovery compared to the asF-iPS group beginning from week 20 post-transplantation (n=5, p<0.05). No recovery was observed in sham group. FIG. 8h shows a representative in vivo Positron Emission Tomography (PET) imaging of the uptake of [18F]PE-P21 ligand to evaluate recovery of dopamine transporter (DAT) function in striatal dopaminergic neurons 6 months following transplantation. Mice transplanted with human EC23-iPS NPCs showed recovery of DAT activity compared to those transplanted with human asF-iPS NPCs or sham controls. Scale bars: 200 μm in FIG. 8a; 100 μm in FIG. 8b-f.

FIG. 9 shows an exemplary in vivo PET imaging of striatal dopamine production in engrafted mice. The PET illustrates the uptake of [18F]PE-P21 ligand to evaluate recovery of dopamine transporter (DAT) function in striatal dopaminergic neurons 6 months after iPS-derived NPCs transplantation. Mice transplanted with human CLEC-iPS-derived NPCs show apparent recovery of DAT activity compared to those transplanted with human adult iPS-derived NPCs or sham transplanted controls.

FIG. 10 illustrates the in vivo maintenance of graft derived from human CLiPS 6 and 9 months after implantation into mice brains. The graft is stained positive for human antigen NCAM and TH dopaminergic marker. A formation of tumors has not been recorded. Scale bars: 50 μm.

FIG. 11 shows the results of a histological and functional analysis of transplanted human EC23-CLiPS dopaminergic NPCs in a Medial Forebrain Bundle (MFB) lesion model of PD created in fully-immunocompetent Wistar Hannover rats. FIG. 11a shows engraftment of human EC23-CLiPS neurons in striatal region of a rat brain 3 months after transplantation demonstrated by positive double-staining for human cytoplasm (STEM 121) and human nuclear antigen (HuNu) antibodies. The staining indicates functional recovery. FIG. 11b indicates colocalization of Synapsin 1 immunoreactivity with hNCAM+/TH+ neurons suggesting possible integration of transplanted human CLiPS-derived cells with host tissues 3 months after transplantation. FIG. 11c shows a retrograde lesioning of the dopaminergic system in the substantia nigra in a rat brain. FIG. 11d shows an unlesioned rat brain confirming the retrograde lesioning of the dopaminergic system in the substantia nigra of FIG. 11c by tyrosine hydroxylase (TH) immunostaining. FIG. 11e shows the result of an Apomorphine-induced rotational asymmetry assay in rats transplanted with dopaminergic NPCs derived from human CLEC23-iPS. The results indicate that transplantation of CLiPS-NPCs mediated recovery of functional motor deficits in a rat MFB model of PD over a 6 months study period. Scale bars: 100 μm in FIG. 11a and FIG. 11b; 200 μm in FIG. 11c and FIG. 11d.

FIGS. 12a, b and c each show exemplary colonies of induced pluripotent stem cells derived from human CLEC that have been generated by using the medium PTTe-3 as recovery medium.

FIG. 13 shows CLiPS differentiate to RPEs: Images of differentiation cultures from different stem cells, human ES cell (H9), iPS cell lines derived from skin (Asf5, AGO, HDFA), umbilical cord-lining mesenchymal cells (CLMC23, CLMC30, CLMC44) and umbilical cord-lining ectodermal cells (CLEC23). Darker patches on the cell culture plates correspond to presence of pigmented RPE cells.

FIG. 14 shows CLMCs have consistently high differentiation efficiency: FIG. 14a shows that a visual grading system for estimating RPE differentiation efficiency based on percentage area of the well occupied by pigmented cells, RPE differentiation efficiency is graded as 0, 1, 2 or 3 for no pigmentation, <30%, 30-60% or >60% pigmentation, respectively. FIG. 14b shows RPE differentiation efficiency estimated by visual grading of pigmented cell area of differentiation plates. Each bar represents grading of one differentiation plate, numbers on the bars denotes percentage of wells on the plate with different grades of pigmentation indicated by different shades of brown. Numbers 1-3 represent biological replicates. FIG. 14c shows RPE differentiation efficiency of different stem cells estimated by Pmel17 by flow cytometry; cells from 3 wells were pooled for FACS analysis.

FIG. 15 shows CLiPS-derived RPEs have more pigmentation compared to ES-derived: FIG. 15a shows images of differentiation plates taken at identical conditions on day 30 of differentiation, using ChemiDoc Touch gel imaging system (Bio-Rad laboratories). FIG. 15b shows phase-contrast Images of RPE from different stem cells exhibiting weak pigmentation of H9. H9: (human ES cell-derived RPE), CLMC23, CLMC30, CLMC44, CLEC23 (RPE derived from CLiPS), AGO, HDFA, Asf5 (RPE derived from skin-iPS cells). FIG. 15c shows CLiPS-derived RPEs have more pigmentation compared to ES-derived. Graph showing darkness of pigmentation analyzed from images of differentiation plates taken using Cmemidoc Touch system from Biorad at different points along differentiation H9: (human ES cell-derived RPE), CLMC23, CLMC30 and CLEC23 (RPE derived from CLiPS). FIG. 15d shows pigmentation-related and RPE specific genes are higher in CLiPs: RT-qCPR analysis of genes involved in pigmentation at day 18 and day 35 of differentiation: MITF, PMEL17, TYROSINASE, TRYP2.

FIG. 16 shows CLiPS expressing RPE specific genes at day 18 and day 35 of differentiation: RT-qCPR analysis of RPE-specific RPE65 and MERTK.

FIG. 17 shows CLiPS derived-RPEs are functional. FIG. 17a shows tight junctions are formed by in vitro generated RPEs, similar to native RPEs: Trans-epithelial electrical resistance (TEER), a measure of tight-junction integrity, in RPEs derived from different stem cells measured over a period of 4 months using Epithelial Volt Ohm meter, EVOM2™. FIG. 17b shows in vitro generated RPEs are highly phagocytic: Percentage phagocytosis of FITC-labelled photoreceptor outer segments (POS) by RPEs derived from different stem cells.

FIG. 18 shows CLiPS derived-RPEs showing protein expression similar to ES-derived. CLiPS-RPEs showing apical expression of Mertk, junctional expression of ZO-1 and cytoplasmic expression of RPE65.

FIG. 19 shows the original method of RPE differentiation and the modifications: FIG. 19a shows the schematic of the original method showing the differentiation mediums used at different stages and their composition. FIG. 19b shows the modifications introduced to the differentiation protocol, showing gradual increase in CHIR99021 concentration and replacement of FGF inhibitor, SU5402 with PD173074. FIG. 19c shows a photo of CLMC30 plate differentiated using the published protocol using SU5402 or modified protocol using PD173074 showing similar degree of RPE differentiation and pigmentation. DM1-DM5: differentiation mediums 1-5. Modified RPE differentiation protocol using PD173074 yields functional RPEs. Functionality of RPEs derived from differentiation methods using SU5405 or PD173074 tested for TEER (FIG. 19d) and phagocytosis of FITC-labelled POS particles (FIG. 19e).

FIGS. 20a and b show comparison of RPE yield by different purification methods. In particular, FIGS. 20a and b show the schematic representation of different methods of RPE purification: Differentiation cultures containing RPE and non-RPE were purified (i) Manual purification: identification of non-RPE cells based on their morphology and lack of pigmentation and manual removal of them by scraping by observing under a dissection microscope, (ii) TrypLE purification: removal of majority of weakly attached non-RPE clusters by partial TrypLE treatment, (iii) TrypLE+ Manual: elimination of majority of weakly attached non-RPE clusters by partial TrypLE treatment followed by manual removal of few non-RPE clusters that escaped TrypLE treatment by observing under a dissection microscope (iv) TrypLE+ scatter sorting: removal of weakly attached non-RPE clusters by partial TrypLE treatment followed by scatter sorting, (v) Scatter sorting: separation of all cells from mixed differentiation culture based on their relative light scatter, as scatter-high (pigmented RPE cells) and scatter-low (non-pigmented non-RPE cells) populations. FIGS. 20c and d show the original and modified scatter soring method to more accurately select the scatter high RPE cells. FIG. 20c shows arbitrarily chosen gates for scatter high (cyan) and low gate (magenta) as in the original protocol. FIG. 20d shows the modified gate selection using weakly attached non-RPE cells dissociated by partial TryPLE treatment to set scatter low gate (magenta) to more accurately select scatter high gate (cyan). FIG. 20e shows yield of RPE obtained from different methods of purification. FIG. 20f shows purity of RPE from different methods of purification assessed by Pmel17 flow cytometry. FIG. 20g shows TEER of RPE obtained from different methods of purification; M: Manual purification, T: TrypLE purification, T+M: TrypLE+manual purification, T+Sc: TrypLE+Scatter sorting, Sc: Scatter sorting, T (loose): weakly attached non-RPE cells easily detached by TrypLE treatment, Sc low: scatter low non-RPE cells from scatter sorting. FIG. 20h shows phagocytic capacity of RPE from different methods of purification assessed by photoreceptor outer segment (POS) phagocytosis assay. FIG. 20i shows table comparing different methods of RPE purification. FIG. 20j shows quantitative PCR comparison of RPE specific gene expression in CLMC23 and H9. qPCR results showing relative expression of RPE-specific genes such as BEST1, RPE65, RLBP1, MERTK, MITF, PMEL17 and TRYP2 normalized to GAPDH. FIG. 20k shows comparison of gene expression in CLMC23 and H9, expressed as fold change of CLMC23 over H9.

FIG. 21 shows that CLiPs-RPE (CLEC23-RPE) has bioenergetic profile similar to native RPE (AHRPE). CLiPsRPE (CLEC23-RPE) also show higher glycolysis and oxidative phosphorylation compared to both skiniPSC-RPE (ASF5-RPE) and hESC-RPE (H9-RPE). FIG. 21a shows that for OCR curve basal respiration, ATP production, maximal capacity and spare respiratory capacity are higher in CLiPs-RPE by 38%, 40%, 35% and 36% respectively compared to H9-RPE. FIG. 21b shows that for ECAR curve glycolysis, glycolytic capacity and glycolytic reserve are higher in CLiPs-RPE by 25%, 37% and 50% respectively compared to H9-RPE. FIGS. 21c-f show that CLiPs-RPE show increased resistance to oxidized low-density lipoprotein (oxLDL) as evidenced by no decrease in maximal capacity after exposure to oxLDL dotted curve) for CLEC23-RPE (c) vs. 27% reduction in ASF5-RPE (d) and 43% reduction in H9-RPE (e). (CLiPs-RPE cells' response to oxidative stress is similar to that seen in native RPE (AHRPE—f) making them functionally closer to primary RPE compared to other differentiated RPE. FIGS. 21g-j show that CLiPs-RPE show increased resistance to hydrogen peroxide (H2O2) as evidenced by no decrease in maximal capacity after exposure to H2O2 (dotted curve) for CLEC23-RPE (g) vs. 27% reduction in ASF5-RPE (h) and 99% reduction in H9-RPE (i). CLiPs-RPE cells' response to oxidative stress is similar to that seen in native RPE (AHRPE) (f & j) making them functionally closer to primary RPE compared to other differentiated RPE.

FIG. 22 shows absence of immune system clearance of all stem cell-derived retinal pigment epithelial (SC-RPE) cell lines. FIGS. 22a and b show in-vivo bioluminescence measurements (total radiance) of injected luciferase-expressing SC-RPEs embedded in matrigel plugs at indicated time points in both humanized and NOD-SCID IL2Rγ−/− (immunodeficient) mice. FIG. 22c shows representative images of all SC-RPE lines showing RPE65, Ki67 and Hoechst staining from the matrigel plugs engrafted in humanized mice at the endpoint of 2 months. Scale bar, 50 μm.

FIG. 23 shows that CLEC23-RPE group has reduced levels of pro-inflammatory cytokines involved in induction of cellular immune response. FIGS. 23a and b show serum cytokines (IFN-γ and IL-18) at the end-point being analyzed. FIG. 23c shows representative images showing OTX2, human CD45 (hCD45) and Hoechst staining of the RPE-matrigel plugs indicating immune cell infiltration. FIGS. 23d and e show cellular immune response grading (0 to 3) based on hCD45 positive cells in the RPE-matrigel plugs. Scale bar, 50 μm.

FIG. 24 shows CLEC23-RPE may suppress CD8 T cell activation. FIGS. 24a and b show serum cytokines (IL-23 and IL-17A) at the end-point being analyzed. FIG. 24c shows that T cell (CD3) to B cell (CD19) ratio was calculated after flow cytometry analysis. FIG. 24d shows that CD3-positive cells were further gated into Helper T (CD4) and Cytotoxic T (CD8) cells to analyze T cell differentiation. FIGS. 24e and f show that CD4-positive and CD8-positive cells were gated into four groups of differing T cell activation status based on specific surface markers.

### DETAILED DESCRIPTION OF THE INVENTION

The present invention is inter alia directed to a method of generating an induced pluripotent stem cell, from a stem cell of the amniotic membrane of the umbilical cord under conditions suitable to reprogram the stem cell, thereby generating the induced pluripotent stem cell (iPS).

In the present invention, both mesenchymal and epithelial stem cells of the amniotic membrane of the umbilical cord—also jointly referred to herein as cord lining stem cells (CLSC) are used to generate iPS—also referred to herein as cord lining-derived induced pluripotent stem cells or “CLiPS”. It has been surprisingly found that cord lining-derived induced pluripotent stem cells of the present invention are robust and homogenous stem cells capable to differentiate into functional target cells of different lineages (cf., Examples 3 and 4). For example, cord lining-derived induced pluripotent stem cells have the capacity to differentiate in multiple cell types and can, for example, be differentiated into various cells types such as hepatocytes representing endodermal tissue (cf., Example 8), cardiomyocytes representing mesodermal tissue (cf., Example 9), and dopaminergic neurons (cf., Example 7) and oligodendrocytes (cf., Example 10) representing ectodermal tissue. Even more surprising and important is the finding that, for example, human CLiPS-derived dopaminergic neurons are able to engraft functionally in different species and survived for up to 9 months in mice Parkinson's disease (PD) models in the absence of immunosuppression and 6 months in rat PD models in the absence of immunosuppression (cf. Examples 12 and 13). Therefore, in summary, the present inventors have generated a hypo-immunogenic cell source that is capable of engrafting, integrating and mediating therapeutic recovery in a fully immunocompetent host. The cord lining-derived induced pluripotent stem cells of the present invention can potentially be used as a universal source of cell for allogeneic cell transplantation in humans without the need for immunosuppression, and this making them ideal candidates for such cell based therapies. As a further advantage it was found here that the cord lining-derived induced pluripotent stem cells of the invention can be generated by an integration- and feeder free method, thereby allowing an iPS production under current good manufacturing practice (cGMP) conditions. Since a GMP process for the production of large amounts of mesenchymal stem cells of the amniotic membrane of the umbilical cord has recently been established (see International Application WO 2018/067071 or US patent application US2018127721), the present invention provides an ideal platform to produce iPS for subsequent cell-based therapy in humans or animals.

In sum, CLiPS derived from very young tissue are less likely to carry genetic, epigenetic and mitochondrial DNA mutations as they are derived from a young tissue. Because of these advantages, CLiPS are a potentially superior stem cell source for generating differentiated cells for regenerative medicine. Hence, they are superior over iPS cells derived from skin or blood which require invasive procedures of tissue collection. They are also free from ethical issues associated with ES cells. Thus, CLiPS are a better source of stem cell for regenerative medicine. The inventors found that such CLiPS robustly differentiate to retinal pigment epithelial (RPE) cells, also called RPEs, by the method of the present invention. For this invention, the inventors compared different stem cell resources: Human ES cells (ES), iPS cells derived from skins (skin-iPS) and cord-lining cells (CLiPS), for their ability to generate RPEs in-vitro. CLiPS can be of either mesenchymal (CLMC) or ectodermal (CLEC) origin. The inventors then compared RPE differentiation efficiency of CLiPS with ES and skin-iPS cells. Compared to skin-iPS, CLiPS gave consistently high RPE differentiation efficiency than skin-iPS cells by visual grading and flow cytometry estimation. Comparison of pigmentation of differentiation cultures visually and by image analysis also showed that CLiPS-derived RPEs have higher pigmentation that ES-derived RPEs. The RPE generated from CLiPS also displayed functional characteristics of RPE after maturation in-vitro suggesting they are a superior source of RPE cells. Additionally, the inventors found by comparing bioenergetics of RPE derived from different stem cells that CLiPS-RPE have higher glycolytic and mitochondrial respiration than ES-derived RPEs. The method of the present invention used of differentiating an induced pluripotent stem (iPS) cell derived from a stem cell of the amniotic membrane of the umbilical cord (CLiPS or CLSC) into a RPE cell has been particularly modified as described herein, which achieves maximum RPE yield.

Describing now first the method of generating an iPS of the present invention, this method may comprise expressing exogenous nucleic acid encoding the proteins OCT3/4, SOX2, KLF4, LIN28 and L-MYC and the p53-shRNA. The nucleic acid encoding OCT3/4 (Sequence ID No: 1), also sometimes referred to as POUSFL, OCT3 or OCT4, encodes for the octamer-binding transcription factor 4. OCT3/4 (Sequence ID No: 2) forms a heterodimer with SOX2 to regulate pluripotency factors in a cell. SOX2 (Sequence ID No: 3), also sometimes referred to as SEY, encodes for the sex determining region Y-box 2 transcription factor (Sequence ID No: 4). When bound to OCT3/4, SOX2 binds to a non-palindromic genomic sequence thus activating the transcription of pluripotent factors in a cell. KLF4 (Sequence ID No: 5), also sometimes referred to as GKLF, encodes for the Krueppel-like factor 4. KLF4 (Sequence ID No: 6) is a zinc finger transcription factor, which functions as a tumor suppressor controlling the GI-to-2 transition of the cell cycle by mediating the tumor suppressor p53. L-MYC (Sequence ID No: 7) encodes for a transcription factor (Sequence ID No: 8) activating the expression of proliferative genes. LIN28 (Sequence ID No: 9) encodes for the RNA-binding protein Lin-28 homolog A (Sequence ID No: 10), which regulates the self-renewal of stem cells. The p53-shRNA (Sequence ID No: 11) encodes for a small hairpin RNA directed to p53, a protein that may regulate the cell cycle by stopping it when the protein accumulates in the cell. To avoid a stopping of the cell cycle by p53, p53-shRNA may silence the expression of p53 posttranscriptional. To generate CLiPS, the exogenous nucleic acids encoding OCT3/4, SOX2, KLF4, LIN28, L MYC and p53-shRNA may be transferred into the CLSC for expression. Alternatively, the proteins OCT3/4, SOX2, KLF4, LIN28, L-MYC and the p53 shRNA may be transferred directly into a CLSC.

As explained above, an induced pluripotent stem cell population of the present invention is obtainable by reprogramming stem cells of the amniotic membrane of umbilical cord. The stem cell of the umbilical cord may be an (isolated) mesenchymal stem cell of the amniotic membrane of the umbilical cord, also referred to as cord lining mesenchymal stem cell (CLMC), or an (isolated) epithelial stem cell of the amniotic membrane of the umbilical cord, also referred to as cord lining epithelial stem cell (CLEC). The CLEC and CLMC used to generate the iPS of the present invention may be derived of any mammalian species, such as mouse, rat, guinea pig, rabbit, goat, horse, dog, cat, sheep, monkey or human, with stem cells of human origin being preferred in one embodiment. Accordingly, also the iPS of the present invention can be derived of any mammalian species, such as mouse, rat, guinea pig, rabbit, goat, horse, dog, cat, sheep, monkey or human, with stem cells of human origin being preferred in one embodiment. In a preferred embodiment CLEC is used to generate the iPS of the present invention.

In case epithelial stem cells of the amniotic membrane of the umbilical cord are used as starting material, these epithelial stem cells, can, for example, be obtained as described in US patent application 2006/0078993 (leading to granted U.S. Pat. Nos. 9,085,755 and 9,737,568) or the corresponding International patent application WO2006/019357. If mesenchymal stem cells of the amniotic membrane of the umbilical cord are used as starting material, they can also be obtained as described in US patent application 2006/0078993 (leading to U.S. Pat. Nos. 9,085,755 and 9,737,568) or the corresponding International patent application WO2006/019357.

It is also possible to use as starting material, a mesenchymal stem cell population as described in the published US application 2018/127721 or the corresponding International Application WO 2018/067071. The mesenchymal stem cell population of International Application WO 2018/067071 has the advantage that 99% or more of the stem cells of this population are positive for the three mesenchymal stem cell markers CD73, CD90 and at the same lack expression of CD34, CD45 and HLA-DR, meaning 99% or even more cells of the mesenchymal stem population International Application WO 2018/067071 express the stem cell markers CD73, CD90 and CD105 while not expressing the markers CD34, CD45 and HLA-DR. This extremely homogenous and well defined cell population is the ideal candidate for clinical trials and cell based therapies since, they for example, fully meet the criteria generally accepted for human mesenchymal stem cells to be used for cellular therapy as defined, for example, by Dominici et al, “Minimal criteria for defining multipotent mesenchymal stromal cells. The International Society for Cellular Therapy position statement”, Cytotherapy (2006) Vol. 8, No. 4, 315-317, Sensebe et al., “Production of mesenchymal stromal/stem cells according to good manufacturing practices: a, review”, Stem Cell Research & Therapy 2013, 4:66), Vonk et al., Stem Cell Research & Therapy (2015) 6:94, or Kundrotas Acta Medica Lituanica. 2012. Vol. 19. No. 2. P. 75-79. Thus, the mesenchymal stem population International Application WO 2018/067071 is the ideal starting material for producing the CLiPS of the present invention under GMP conditions.

It is noted in this context that CLMCs transfected with a transgene will maintain their stemness and stem cell characteristics but may show a decrease in the percentage of cells expressing mesenchymal stem cell markers such as CD73, CD90 and CD105 while at the same time may also show an increase in the percentage of cells expressing negative markers such as CD34, CD45 or HLA-DR. See, Yap et al., Malaysian J Pathol 2009; 31(2): 113-120); cf. also Madeira et al, Journal of Biomedicine and Biotechnology. Volume 2010, Article ID 735349, 12 pages. In light of this, it may be possible that a CLiPS of the present invention that has been generated by reprogramming of a CLMC described herein and isolated from the amniotic membrane of the umbilical cord, may be a stem cell population, wherein at least about 81% or more, about 82% or more, at least 83% or more, at least 84% or more, at least about 85% or, about 86% or more, about 87% or more, about 88% or more, about 89% or more, about 90% or more, about 91% or more, about 92% or more, about 93% or more, about 94% or more, about 95% or more, about 96% or more, about 97% or more, about 98% or more about 99% or more cells of the CLiPS population may express each of the following markers: CD73, CD90 and CD105. In addition, such a CLMC derived population of induced pluripotent stem cells of the invention may be a population, wherein at least about 81% or more, about 82% or more, at least 83% or more, at least 84% or more, at least about 85% or, about 86% or more, about 87% or more, about 88% or more, about 89% or more, about 90% or more, about 91% or more, about 92% or more, about 93% or more, about 94% or more, about 95% or more, about 96% or more, about 97% or more, about 98% or more about 99% may lack expression of each of CD34, CD45 and HLA-DR. One preferred example of such CLMC derived population of induced pluripotent stem cells of the invention may be a population, in which at least about 90% or more, about 91% or more, about 92% or more, about 93% or more, about 94% or more, about 95% or more, about 96% or more, about 97% or more, about 98% or more about 99% or more cells of the CLMC population express each of CD73, CD90 and CD105 and lack expression of each of CD34, CD45 and HLA-DR.

Turning again to the generation of an induced pluripotent stem cell (population) of the invention, it is again important to note that such an induced pluripotent stem cell is obtainable by any suitable method that reprograms a stem cell (population) of the amniotic membrane of umbilical cord into such an induced pluripotent stem cell (population). While one method of generating such an induced pluripotent stem cell comprises expressing exogenous nucleic acids encoding the proteins OCT3/4, SOX2, KLF4, LIN28 and L-MYC and the p53-shRNA in a stem cell of the amniotic membrane of the umbilical cord under conditions suitable to reprogram the stem cell, thereby generating the induced pluripotent stem cell, the invention is by no means restricted to CLiPS obtained by this method. Rather, the CLiPS can be obtained by any suitable method as, for example described in the review of Cieslar-Probuda et al “Transdifferentiation and reprogramming: Overview of the processes, their similarities and differences” BBA—Molecular Cell Research, Volume 1864, Issue 7, July 2017, Pages 1359-1369. For example, the reprogramming may be performed in the present invention also chemically by using small molecules or biologically by expressing exogenous nucleic acids encoding for reprogramming factors within a cell. Alternatively, the exogenous nucleic acids encoding the proteins OCT3/4, SOX2, KLF4, LIN28, L-MYC and the p53 shRNA may be provided as any suitable nucleic acid for expression. For example, the nucleic acid may be deoxyribonucleic acid (DNA), ribonucleic acid (RNA) comprising messenger RNA (mRNA) and microRNA (miRNA). The exogenous nucleic acids may be transferred as such or the exogenous nucleic acids may be incorporated into one or more vector(s) suitable to be transferred into a cell. In this context, any vector suitable to be transferred into CLSC can be used. An illustrative example for such a vector may be a plasmid. In the present invention, the exogenous nucleic acids may be provided by one, two, three or four vectors suitable to be transferred into a stem cell. In an illustrative example, three vectors may provide the exogenous nucleic acids for reprogramming CLSC into CLiPS, wherein the vectors may be pCXLE-hOCT3/4-shp53-F (Addgene plasmid #27077; Sequence ID No: 12), pCXLE-hSK (Addgene plasmid #27078, Sequence ID No: 13) and pCXLE-hUL (Addgene plasmid #27080; Sequence ID No: 14).

In accordance with the above, any method suitable for transferring exogenous nucleic acids or a protein into the CSLC can be used. In one example, a viral vector may be used to transfer the exogenous nucleic acid into the CSLC. An example for such a viral vector may be a retrovirus, a lentivirus, an inducible lentivirus, a sendai virus or an adeno virus. Alternatively, transfection may be performed to transfer the exogenous nucleic acids into CSLC. In the present invention, transfection may comprise electroporation, microinjection, liposome- and non-liposome-mediated transfection and sonoporation.

In a preferred example, the CLSC may be subjected to electroporation, wherein the electric parameters may be adjusted depending on the type of CLSC being used, as a CLMC may require different electroporation conditions than a CLEC. The electric parameters may comprise the number of electric pulses applied to the stem cell, the duration time of the applied electric pulse(s) and the voltage of the applied electric pulse(s). Each electric parameter may be adjustable to further optimize the electroporation of the present invention. When so doing, each electric parameter may be adjusted independently or in combination with one or more of the other electric parameter(s) (cf. Example 1). In the present invention, any parameter setting suitable for allowing the transfer of exogenous nucleic acid into CLSC may be applied. In one example of the present invention, a CLMC may be subjected to electroporation. In such a case, the electroporation may be carried out with 1 electric pulse which may have a duration time of about 15 milliseconds (ms) to about 25 ms and a voltage of about 1550 V to about 1650 V. Accordingly, in one example, a CLMC may be subjected to electroporation with 1 electric pulse, which may have a duration time of about 20 ms and a voltage of about 1600 V. In addition, it has been found herein that electroporation yielding usable amounts/numbers of CLiPS derived from CLMC depends on the ratio of each vector (plasmid) DNA transfected to the number of CLMC used for the transfection. This ratio is expressed herein by the amount of each vector (plasmid) DNA (in μg) that is used to the number of CLMC (in 1×10⁶ cell) subjected to electroporation. In illustrative examples, the ratio of the amount of vector (plasmid) DNA for each vector to the number of cells may be in the range of 1.5 μg DNA to about 1×10⁶ CLMC to about 2.5 μg DNA to about 1×10⁶ CLMC. Thus, this ratio may be about 2.5 μg DNA to about 1×10⁶ CLMC, about 2.25 μg DNA to about 1×10⁶ CLMC, about 1.8 μg DNA to about 1×10⁶ CLMC, about 1.7 μg DNA to about 1×10⁶ CLMC, about 1.67 μg DNA to about 1×10⁶ CLMC, about 1.6 μg DNA to about 1×10⁶ CLMC, or about 1.5 μg DNA to about 1×10⁶ CLMC (cf. Table 1 showing that using a ratio of the amount of vector (plasmid) DNA for each plasmid to the number of cells of about 1.67 μg DNA to about 1×10⁶ CLMC yielded an effective transformation yield). Thus, in one embodiment of generating CLiPS derived from CLMC, it is preferred that each of the vectors is used in the same amount in the electroporation of the CLMC. Also a CLEC may be subjected to electroporation to yield CLiPS of the invention. In case of CLiPS derived from CLEC, the electroporation may be carried out with 2 electric pulses, which may each have a duration time of about 25 ms to about 35 ms and a voltage of about 1300 V to about 1400 V each. Accordingly, in one example, a CLEC may be subjected to electroporation with 2 electric pulses, which may have a duration time of about 30 ms and a voltage of about 1350 V each. As for CLMC, it has also been found for CLiPS derived from CLEC that electroporation yielding usable amounts/numbers of CLiPS derived from CLEC depends on the ratio of the amount of each plasmid DNA transfected to the number of CLEC used for the transfection. Also this ratio is expressed herein by the amount of vector (plasmid) DNA (in μg) that is used for transfection to the number of CLEC (in 1×10⁶ cells) which is to be transfected. In illustrative examples, the ratio of the amount of vector (plasmid) DNA to the number of cells may be in the range of about 1.5 μg DNA to about 1×10⁶ CLEC to about 2.5 μg DNA to about 1×10⁶ CLEC. Thus, the ratio may be about 1.5 μg DNA to about 1×10⁶ CLEC, about 1.6 μg DNA to about 1×10⁶ CLEC, about 1.67 μg DNA to about 1×10⁶ CLEC, about 1.7 μg DNA to about 1×10⁶ CLEC, about 1.8 μg DNA to about 1×10⁶ CLEC, about 1.9 μg DNA to about 1×10⁶ CLEC, about 2.0 μg DNA to about 1×10⁶ CLEC, or about 2.5 μg DNA to about 1×10⁶ CLEC (cf. Table 1 showing that using a ratio of the amount of plasmid DNA for each vector to the number of cells of about 1.67 μg DNA to about 1×10⁶ CLEC provided an effective transformation yield). Thus, in one embodiment of generating CliPS derived from CLEC, it is preferred that each of the vectors is used in the same amount in the electroporation of the CLEC. The electroporation of both CLEC and CLMC may be performed in the method of the invention in a uniform electrical field. Thereby, critical consequences of the electroporation such as pH change, ion formation or heat generation may be minimized. The uniform electric field may be generated by maximizing the gap between the electrodes while minimizing the surface area of each electrode. An illustrative example for a system providing such a uniform electric field is the Neon™ Transfection System of ThermoFisher Scientific. Another example of a suitable commercial transfection system is The Gene Pulser MXcell electroporation system, available from Bio-Rad. As a final remark, the transfection can be carried using any suitable electroporation buffer. In case a commercial transfection system such as the Neon™ Transfection System is used, the respective electroporation buffer provided by the manufacturer of the transfection system is typically used for electroporation.

After transfection, the stem cells may be transferred into a medium suitable for cell recovery and cell cultivation. In the present invention, any cell culture medium suitable for cell recovery and/or proliferation can be used. Illustrative examples for such a suitable cell culture medium may be commonly used media for cultivation (propagation) of human induced pluripotent stem cell such as mTeSR1, StemMACS™ iPS-Brew XF, TeSR™-E8, mTeSR™Plus, TeSR™2, mTeSR™1. It is also possible to use for the cell recovery cultivation any medium that capable of supporting proliferation (without differentiation)/healthy growth of CLEC or CLMC. Examples of suitable media for this cultivation of CLEC are, for example, described in US patent application 2006/0078993 and include EpiLife medium, Medium 171, MEGM-Mammary Epithelial Cell Medium or mixtures of such media such as the medium PTT-e3 (that has been used herein for the generation of CLiPS derived from CLEC and that is described herein in detail below). Examples of suitable media for this cultivation of CLMC are, for instance, described in US patent applications 2006/0078993 and 2018/127721 as well as in International Patent Application WO2007/046775 and include DMEM/10% FBS, DMEM:F12 culture medium (a 1:1 mixture of DMEM and Ham's F-12 medium), or a media such as PPT-6 (a culture medium comprising DMEM, F12-medium, Medium 171 and FBS, see US application 2018/127721) or PTT4 (wherein the latter has been used in the Example Section herein for the generation of CLiPS derived from CLMC). It is also possible to use for this cell recovery cultivation mixtures of these media (for example a mixture of mTeSR1 with the medium PTTe-3 or the medium PTT-4). The medium suitable for cell recovery of transfected CLEC or CLMC as described herein may further contain growth factors, which may stimulate cellular growth and proliferation. The growth factors may be added to the cell culture medium as such. In addition, the recovery medium may contain serum such as, for example, fetal bovine serum (FBS). Thus, the medium suitable for cell recovery after transfection may be a serum-free or a serum-containing medium.

In line with the above disclosure, the composition of the medium suitable for cell recovery may differ, depending on the CLSC being used.

For example, the medium suitable for the recovery of a transfected CLMC may consist of a (chemically) defined medium and FBS. Accordingly, the medium suitable for the recovery of a transfected CLMC may consist of about 80% (v/v), about 85% (v/v), about 90% (v/v) or about 95% (v/v) chemically defined medium and about 20% (v/v), about 15% (v/v), about 10% (v/v) or about 5% (v/v) FBS, respectively. In a preferred example, CLMC are cultivated in medium PTT-4 for cell recovery after transfection, wherein medium PTT-4, as described in International Patent Application WO2007/046775, consists of 90% (v/v) CMRL-1066 and 10% (v/v) FBS. A medium suitable for the recovery of a transfected CLEC may be a serum-free medium, wherein the medium may contain cytokines and growth factors.

Also the medium suitable for the recovery of a transfected CLEC may be a defined medium. Such a recovery medium may comprise Mammary Epithelial Basal Medium MCDB 170, EpiLife medium, DMEM (Dulbecco's modified eagle medium), F12 (Ham's F12 Medium) and FBS (Fetal Bovine Serum).

In illustrative examples, such a medium comprises Mammary Epithelial Basal Medium MCDB 170 in a final concentration of about 10 to about 30% (v/v), EpiLife medium in a final concentration of about 20 to about 40% (v/v), F12 in a final concentration of about 5 to about 15% (v/v), DMEM in a final concentration of about 30 to about 45% (v/v) and FBS in a final concentration of about 0.1 to 2% (v/v). One such medium may comprise Mammary Epithelial Basal Medium MCDB 170 in a final concentration of about 15 to about 25% (v/v), EpiLife medium in a final concentration of about 25 to about 35% (v/v), F12 in a final concentration of about 7.5 to about 13% (v/v), DMEM in a final concentration of about 35 to about 40% (v/v) and FBS in a final concentration of about 0.5 to 1.5% (v/v). Another such medium may comprise Mammary Epithelial Basal Medium MCDB 170 in a final concentration of about 20% (v/v), EpiLife medium in a final concentration of about 30% (v/v), F12 in a final concentration of about 12.5 (v/v), DMEM in a final concentration of about 37.5% (v/v) and FBS in a final concentration of about 1.0% (v/v). The value of “% (v/v)” as used herein refers to the volume of the individual component relative to the final volume of the culture medium. This means, if DMEM is, for example, present in the culture medium a final concentration of about 35 to about 40% (v/v), 1 liter of culture medium contains about 350 ml to 400 ml DMEM. In one embodiment, the medium suitable for the recovery of a transfected CLEC cell is obtained by mixing to obtain a final volume of 1000 ml culture medium:
200 ml Mammary Epithelial Basal Medium MCDB 170,
300 ml EpiLife medium,
250 ml DMEM,
250 ml DMEM/F12, and
1% Fetal Bovine Serum.

The growth factors in the medium suitable for the recovery of a transfected CLEC may an insulin like growth factor (IGF) such as IGF-1 or IGF-2, an epidermal growth factor (EGF) such as HB-EGF or EPR, a transforming growth factor (TGF) such as TGF-α or TGF-β 1, an activin, a bone morphogenic protein (BMP), a platelet derived growth factor (PDGF), transferrin and insulin. In one example, CLEC are cultivated in medium PTTe-3 for cell recovery after transfection, wherein medium PTTe-3 contains human epidermal growth factor (EGF), one or more transforming Growth Factors such as TGF-alpha and/or TGF-beta (TGF-beta 1, TGF-beta 2 and/or TGF-beta 3), or insulin.

In accordance with the above, the medium suitable for the recovery of a transfected CLEC may comprise human epidermal growth factor (EGF) in a final concentration of about 1 to about 15 ng/ml. The recovery medium may also comprise insulin in a final concentration of about 1 to about 7.5 μg/ml. This recovery medium may further comprise at least one of the following supplements: adenine, hydrocortisone, and 3,3′,5-Triiodo-L-thyronine sodium salt (T3). In one embodiment the medium comprises all three of adenine, hydrocortisone, and 3,3′,5-Triiodo-L-thyronine sodium salt (T3). In this case, the medium may comprise adenine in a final concentration of about 0.05 to about 0.1 mM adenine, hydrocortisone in a final concentration of about 0.1 to 0.5 μM hydrocortisone and 3,3′,5-Triiodo-L-thyronine sodium salt (T3) in a final concentration of about 0.1 to about 5 ng/ml. The recovery medium may comprise one of more transforming growth factors (TGF), for example transforming growth factor beta 1 (TGF-beta 1) and/or transforming growth factor alpha (TGF-alpha. In such a medium, TGF-beta 1 may be present in a final concentration of about 0.1 to about 5 ng/ml and TGF-alpha may be present in a final concentration of about 1.0 to about 10 ng/ml. In addition, the medium of recovery of CLEC may comprise Cholera Toxin from Vibrio cholerae (which is commercially available, for example, from Sigma Aldrich under catalogue number C8052. If cholera toxin from Vibrio cholerae is used, it may be present in a final concentration of about 1×10⁻¹¹M to about 1×10⁻¹⁰M.

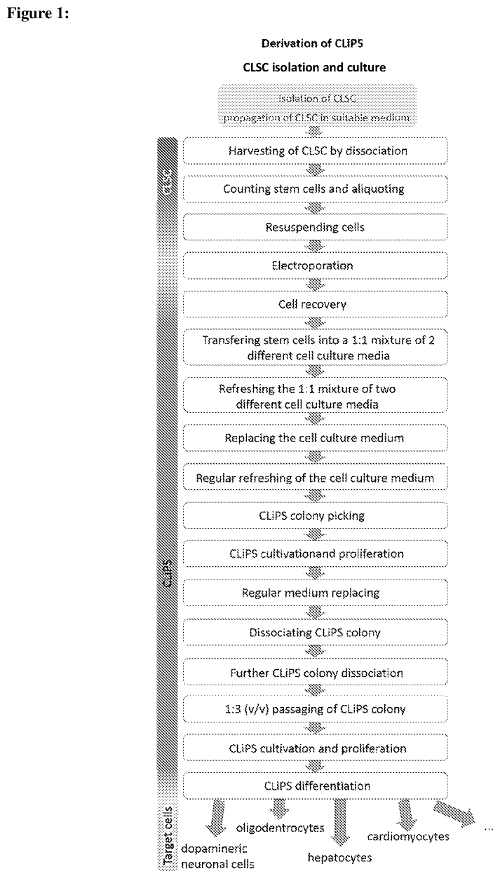

By “DMEM” is meant Dulbecco's modified eagle medium which was developed in 1969 and is a modification of basal medium eagle (BME) (cf. FIG. 1 showing the data sheet of DMEM available from Lonza). The original DMEM formula contains 1000 mg/L of glucose and was first reported for culturing embryonic mouse cells. DMEM has since then become a standard medium for cell culture that is commercially available from various sources such as ThermoFisher Scientific (catalogue number 11965-084), Sigma Aldrich (catalogue number D5546) or Lonza, to name only a few suppliers. Thus, any commercially available DMEM can be used in the present invention. In preferred embodiments, the DMEM used herein is the DMEM medium available from Lonza under catalog number 12-604F. This medium is DMEM supplemented with 4.5 g/L glucose and L-glutamine. In another preferred embodiment the DMEM used herein is the DMEM medium of Sigma Aldrich catalogue number D5546 that contains 1000 mg/L glucose, and sodium bicarbonate but is without L-glutamine.

By “F12” medium is meant Ham's F12 medium. This medium is also a standard cell culture medium and is a nutrient mixture initially designed to cultivate a wide variety of mammalian and hybridoma cells when used with serum in combination with hormones and transferrin. Any commercially available Ham's F12 medium (for example, from ThermoFisher Scientific (catalogue number 11765-054), Sigma Aldrich (catalogue number N4888) or Lonza, to name only a few suppliers) can be used in the present invention. In preferred embodiments, Ham's F12 medium from Lonza is used. By “DMEM/F12” or “DMEM:F12” is meant a 1:1 mixture of DMEM with Ham's F12 culture medium. Also DMEM/F12 (1:1) medium is a widely used basal medium for supporting the growth of many different mammalian cells and is commercially available from various supplier such as ThermoFisher Scientific (catalogue number 11330057), Sigma Aldrich (catalogue number D6421) or Lonza. Any commercially available DMEM:F12 medium can be used in the present invention. In preferred embodiments, the DMEM:F12 medium used herein is the DMEM/F12 (1:1) medium available from Lonza under catalog number 12-719F (which is DMEM: F12 with L-glutamine, 15 mM HEPES, and 3.151 g/L glucose).

By “M171” is meant culture medium 171, which has been developed as basal medium for the culture of for the growth of normal human mammary epithelial cells. Also this basal medium is widely used and is commercially available from supplier such as ThermoFisher Scientific or Life Technologies Corporation (catalogue number M171500), for example. Any commercially available M171 medium can be used in the present invention. In preferred embodiments, the M171 medium used herein is the M171 medium available from Life Technologies Corporation under catalogue number M171500.

By “Mammary Epithelial Basal Medium MCDB 170” is meant a basal nutrient medium that is used for the growth of mammary epithelial cells and that is commercially available in powder form, for example, from United States Biological, Salem Massachusetts USA under catalogue number M2162 or from Bio-Connect B.V., Huissen, The Netherlands, under catalogue number (MBS652676_10l)

By EpiLife medium is meant a HEPES and bicarbonate buffered liquid medium that is prepared without calcium chloride and that is commonly used for the long-term, serum-free culture of human epidermal keratinocytes and human corneal epithelial cells and is designed for use in an incubator with an atmosphere of 5% CO2 and 95% air. Available from ThermoFisher Scientific, catalogue number MEPICF500 or from Sigma Aldrich under Product Code E 0151.

By “CMRL medium” is meant the medium that was originally developed by Connaught Medical Research Laboratories for the growth of Earle's ‘L’ cells under serum-free conditions. CMRL medium is known to be also especially useful for cloning monkey kidney cells and for growth of other mammalian cell lines when supplemented with horse or calf serum. CMRL medium is commercially available, for example, from ThermoFisher Scientific (catalogue number 11530037)

By “FBS” is meant fetal bovine serum (that is also referred to as “fetal calf serum”), i.e. the blood fraction that remains after the natural coagulation of blood, followed by centrifugation to remove any remaining red blood cells. Fetal bovine serum is the most widely used serum-supplement for in vitro cell culture of eukaryotic cells because it has a very low level of antibodies and contains more growth factors, allowing for versatility in many different cell culture applications. The FBS is preferably obtained from a member of the International Serum Industry Association (ISIA) whose primary focus is the safety and safe use of serum and animal derived products through proper origin traceability, truth in labeling, and appropriate standardization and oversight. Suppliers of FBS that are ISIA members include Abattoir Basics Company, Animal Technologies Inc., Biomin Biotechnologia LTDA, GE Healthcare, Gibco by Thermo Fisher Scientific and Life Science Production, to mention only a few. In currently preferred embodiments, the FBS is obtained from GE Healthcare under catalogue number A15-151.

The medium suitable for cell recovery may also contain a compound, which may suppress an inflammatory response and/or may also enhance cell survival and proliferation after transfection. An illustrative example for such a compound may be a glucocorticoid. Glucocorticoids are steroid hormones, which are able to up-regulate the expression of anti-inflammatory proteins in the nucleus and repress the expression of pro-inflammatory proteins in the cytosol. The glucocorticoid used herein may be prednisolone, methylprednisolone, dexamethasone, betamethasone, corticosterone or hydrocortisone, to name only a few illustrative examples of suitable glucocorticoids. It is also possible to use two or more such of glucocorticoids together, for example, a mixture of corticosterone and hydrocortisone. The glucocorticoid can be used in any suitable concentration, for example, in a concentration of about 0.1 μM to about 2.5 μM or to about 5 μM. In one illustrative example, the glucocorticoid in the medium suitable for the recovery of transfected CLSC may be hydrocortisone used in a concentration of about 0.1 μM to about 2.5 μM. In one example, the hydrocortisone concentration in the medium suitable for the recovery of transfected CLSC is about 0.5 μM to about 2 μM. In one such illustrative example, the hydrocortisone concentration is about 1 μM.

The recovery of the transfected CLSC may be carried out in a cell culture device such as a cell culture vessel. The cell culture vessel may be, but is not limited to, a cultivation flask, a petri dish, a roller bottle and a multiwall plate. Further, the cell culture vessel may be coated to provide a layer, which may facilitate the cell growth by supplying the cells with metabolites. The coating of the cell culture vessel may be serum-derived or serum-free. An example for a serum-derived coating may be a coating with gelatinous proteins from the basement membrane-like matrix such as Matrigel. A serum-free coating of the cell culture vessel may instead be characterized by being animal and xeno-free thus allowing a cell cultivation under cGMP conditions. An example for a serum-free coating of the cell culture vessel may be a coating with recombinant proteins or parts thereof such as, for example, a coating with a extracellular matrix protein such as collagen, fibronectin, elastin, laminin, including, for instance, the laminin-511 E8 fragment, or laminin 521, vitronectin, for example, in the form of commercially available citronectin XF™, CELLstart or the Synthemax™ vitronectin substrate. In one example of the present invention transfected CLEC may be preferably cultivated in a cell culture vessel with a serum-derived coating, whereas CLMC may be preferably cultivated in a cell culture vessel with a serum-free coating.

The medium suitable for the recovery of the transfected CLSC may be replaced with another cell culture medium after a suitable period of time. The suitable period of time may, for example, be about 1, about 2 or about 3 days after transfection. Thus, in one example, the medium replacement may be carried out about 2 days after transfection. Another cell culture medium used for the medium replacement may also be a mixture of different cell culture media. In the present invention, any cell culture medium or cell culture medium mixture suitable for yielding of iPS can be used. Furthermore, the suitable cell culture medium or cell culture media mixture may contain a compound, which may suppress inflammatory response and enhance cell survival. In the present invention the medium suitable for cell recovery after transfection may be replaced with a mixture of two different cell culture media after a suitable period of time to ensure a proper supply of nutrients and a suitable blend of growth factors to the cells as they transition from their native state into a more pluripotent state when undergoing somatic reprogramming. Accordingly, the cell culture media mixture of the present invention may consist of the medium suitable for cell recovery, which may contain hydrocortisone, and a second cell culture medium. In a preferred example, the two different cell culture media are mixed in a ratio of about 1:1 (v/v), wherein the mixture may be prepared by contacting 1 volume medium suitable for cell recovery to 1 volume second cell culture medium. In a another preferred example, the two different cell culture media are mixed in a ratio of about 1:2 (v/v) or 2:1, wherein the mixture may be prepared by contacting 1 volume medium suitable for cell recovery to 2 volumes second cell culture medium (or 2 volumes medium suitable for cell recovery to 1 volumes second cell culture medium) The second cell culture medium used for generating the cell culture mixture may be any cell culture medium suitable to enhance or maintain iPS proliferation (such medium is also termed “maintenance medium” herein). Using a mixture such a 1:1 mixture of the medium used for cell recovery and the maintenance medium provides the advantage of allowing the CLiPS cells to transition gradually from their cognate culture medium to the ES/iPSC medium, instead of a sudden switch that might compromise their viability. Without wishing to be bound by theory, it is assumes that about two days after transfection, some successfully transfected cord lining stem cells will start to acquire pluripotent stem cell characteristics and concomitantly, acquire nutrient requirements of PSCs. Illustrative examples for such a suitable cell culture medium include, but are not limited to, commercially maintenance media such as mTeSR1, StemMACS™ iPS-Brew XF, TeSR™-E8, mTeSR™Plus, TeSR™2 or mTeSR™1, Corning@NutriStem® hPSC XF Medium, Essential 8 Medium (ThermoFisher Scientific), StemFlex (ThermoFisher Scientific), StemFit Basic02 (Ajinomoto Co. Inc), or PluriSTEM (Merck Millipore). The medium mTeSR™1, since being manufactured under GMP conditions may be preferably used, if the iPS colonies are cultivated under animal- and xeno-free GMP conditions. Thus, in one preferred example, mTeSR1 may be the second cell culture medium used for generating the cell culture mixture. In the present invention, the 1:1 (v/v) cell culture media mixture may be replaced with the same mixture of cell culture media within a suitable period of time. This suitable period of time may be about 3, about 4, about 5 or about 6 days after transfection. Thus, in one example, the 1:1 (v/v) cell culture media mixture may be replaced with the same mixture 4 days after transfection. After a suitable period of time, the 1:1 (v/v) cell culture media mixture may be further replaced with the second cell culture medium used for generating the cell culture mixture only. In this context, a suitable period of time may be about 4, about 5, about 6 or about 7 days after transfection. In one example, the 1:1 (v/v) cell culture media mixture may be replaced with the second cell culture medium 6 days after transfection. In a preferred example, the 1:1 (v/v) cell culture media mixture may be replaced with mTeSR1 and mTeSR™1, respectively, 6 days after transfection. The regular cell culture media changes and replacements may contribute to an increase of surviving CLiPS. Thus, CLiPS colonies may grow and proliferate.

After changing the cell culture media mixture to one cell culture medium, the CLiPS may be further cultivated. For this purpose, the cell culture medium may also be replaced regularly with the same medium to ensure a proper supply of nutrients and a suitable blend of growth factors to the cells. For example, the cell culture medium may be replaced daily or every second day, every third day or every fourth day. In one example of the present invention, the cell culture medium may be replaced every second day. Consequently, CLiPS colonies may further grow and proliferate.

CLiPS colonies may become visible to the naked eye about 10, 11, 12, 13, 14, 15, or 16 days after transfection (cf., Example 2). When reaching a suitable size, CLiPS may be selected and transferred to another coated culture vessel for further cultivation and proliferation. In this context, a suitable colony size may comprise a length of about 0.1 mm to about 2 mm in diameter. In one example of the present invention, the CLiPS colony may be selected when reaching a length of about 0.5 mm to about 1.5 mm in diameter, wherein the CLiPS colonies may reach this size about 20 days after transfection. To transfer a CLiPS colony with a suitable size to another cultivation vessel, the CLiPS colony may be picked. This may be carried out manually, if wanted. To facilitate the colony picking, a device allowing an enlarged view of the colonies may be used. Examples for such a device may be a magnifier or a microscope. In the present invention, the CLiPS may be selected and picked under bright field microscopy. Turning to the cell culture vessel, the picked CLiPS colonies may be transferred to another cell culture vessel, wherein the coating of the cell culture vessel may vary from the coating of the cell culture vessel used for the recovery of the transfected CLSC or it may be the same. In a preferred example, the coating of the culture vessel is the same, since CLMC-derived CLiPS thus far cultivated under cGMP suitable conditions may be maintained animal- and xeno-free, thereby preserving cGMP conditions. Consequently, a CLMC-derived CLiPS colony, for example, may be transferred in a cell culture vessel coated with a serum-free substance such as the laminin-511 E8 fragment for further cultivation (cf., Example 3). Alternatively, a CLEC- and/or CLMC-derived CLiPS colony, for example, may be transferred in a cell culture vessel coated with a serum-derived substance such as Matrigel for further cultivation. The cell culture medium may preferably be the same as used before the colony picking. In the examples of the present invention, the cell culture medium may be also replaced regularly after colony picking. For example, medium may be replaced daily, every second day or every third day. In a preferred example of the present invention, the cell culture medium may be replaced daily after colony picking.

When reaching a suitable confluence, the CLiPS colonies or a cell population formed from the colonies are typically detached from the coated cell culture vessel and transferred to a larger cell culture vessel for further cultivation under the same cultivation conditions used directly after the colony picking. A suitable confluence may be at least about 40%, at least about 45%, at least about 50%, at least about 55%, at least about 60% and at least about 65% confluence. It is noted in this context that the term “cell population” when used in relation to the propagation of CLiPS forming colonies is more suitable as the CLiPS cells do not take on a colony-like appearance when they reach a confluence of about 70% to about 80%. For detaching the CLiPS colonies or a cell population formed from the colonies from the coated cell culture vessel, any dissociation agent suitable to disrupt cell adhesion or hydrolyze peptide bonds can be used. An example for such a suitable dissociation agent may be a solution containing a chelating agent such as ethylenediaminetetraacetic acid (EDTA) or a solution containing an enzyme such as trypsin or dispase (see the experimental section of the present application, in which dispase has been used to detach a CLiPS colony from the coated cell culture vessel). The cell culture medium may also be replaced regularly, for example, daily, every second day or every third day. In a preferred example of the present invention, the cell culture medium may be replaced daily. This way, the CLiPS may further grow and proliferate.

In the present invention, a CLiPS colony or a cell population formed from a colony may be passaged when reaching a suitable size. The suitable size may correspond to about 50%, about 55%, about 60%, about 65%, about 70%, about 75%, about 80%, about 85%, about 90% and about 95% confluence. In an example of the present invention, the CLiPS colony or the cell population formed therefrom may be passaged when the culture reaches about 60-90% confluence. Thus, in a preferred example, the CLiPS colony or the cell population formed therefrom may be passaged when reaching about 70-80% confluence. For passaging, CLiPS may be passaged in a suitable ratio, wherein one volume CLiPS may be contacted with multiple volumes of cell culture medium. In the present invention, CLiPS may be passaged in a ratio of about 1:3 (v/v), or about 1:4 (v/v), or about 1:5 (v/v) or about 1:6 (v/v), wherein the passaging may be performed by dividing 1 volume dissociated CLiPS into about 2, or about 3, or about 4 or about 5 volumes of dissociated CLiPS, respectively. In a preferred example, CLiPS may be passaged in a ratio of about 1:3 (v/v). To allow passaging of cultivated CLiPS of the present invention, again any enzyme suitable to detach the cells from the culture vessel can be used. For example, dispase may be used for this purpose. Further, any chemical suitable to remove cell-to-cell adhesion can be used for CLiPS passaging in the context of the present invention, wherein the concentration of the chemical may be suitable to remove cell-to-cell adhesion without harming the cells. An illustrative example for such a chemical may be EDTA. Since EDTA may kill cells at higher concentrations, a suitable EDTA concentration of the present invention may be about 0.5 mM. In the present invention, the cell culture medium used for passaging may be supplemented with a substance suitable for enhancing the survival of the CLiPS when dissociated. For this purpose, any substance suitable for enhancing the survival of the CLiPS when dissociated may be used. An example of such a suitable substance may be an inhibitor of a signaling pathway such as the rho-associated protein kinase (ROCK) signalling pathway. Thus, the RHO/ROCK pathway inhibitor Y-27632 may be an illustrative example for a substance suitable for enhancing the survival of dissociated CLiPS. Alternatively, a defined supplement for single-cell cloning of human iPS cells such as CloneR™ (available from StemCell Technologies) may also be used for enhancing the survival of the dissociated cells. In the present invention, the passaged CLiPS may be cultivated in a medium supplemented with the substance suitable for enhancing the survival of the dissociated CLiPS for a suitable period of time before getting differentiated into a target cell.

By cultivating CLiPS after passaging, a master cell bank containing (primary) isolated CLiPS can be obtained. For generating a master cell bank of CLiPs, CLiPS cells obtained by the process as described herein can be seeded in a cultivation vessel such as a cell culture plate. CLiPS can, for this purpose, be suspended and cultured in any suitable medium, typically a maintenance medium for iPS cells such as commercially media mentioned above such as mTeSR1, StemMACS™ iPS-Brew XF, TeSR™ E8, mTeSRTMPlus, TeSRTM2 or mTeSRTM1, Corning® NutriStem® hPSC XF Medium, Essential 8 Medium (ThermoFisher Scientific), StemFlex (ThermoFisher Scientific), StemFit Basic02 (Ajinomoto Co. Inc), or PluriSTEM (Merck Millipore). Both CLiPS derived from CLMC and CliPS derived from CLEC can be cultivated in such a iPS maintenance medium. For subculturing, the CLiPS cells (of both CLMC- and CLEC derived CLiPS) can be seeded at any suitable concentration, for example, or a concentration of about 0.5×10⁶ cells/ml to about 5.0×10⁶ cells/ml. In one example, the cells are suspended for subcultivation at a concentration of about 1.0×10⁶ cells/ml. The subculturing can be carried by cultivation either in simple culture flasks but also, for example, in a multilayer system such as CellSTACK (Corning, NY, USA) or Cell Factory (Nunc, part of Thermo Fisher Scientific Inc., Waltham, MA, USA) that can be stacked in incubators. Alternatively, the subculturing can also be carried out in a closed self-contained system such as a bioreactor. Different designs of bioreactors are known to the person skilled in the art, for example, parallel-plate, hollow-fiber, or micro-fluidic bioreactors. See, for example, Sensebe et al. “Production of mesenchymal stromal/stem cells according to good manufacturing practices: a review”, supra. An illustrative example of a commercially hollow-fiber bioreactor is the Quantum® Cell Expansion System (Terumo BCT, Inc). that has, for example, been used for the expansion of bone marrow mesenchymal stem cells for clinical trials (cf., Hanley et al, Efficient Manufacturing of Therapeutic Mesenchymal Stromal Cells Using the Quantum Cell Expansion System, Cytotherapy. 2014 August; 16(8): 1048-1058) and for the expansion of the highly pure cord ling mesenchymal stem cell population described in International Patent Application WO 2018/067071. Another example of commercially available bioreactors that can be used for the subculturing of the CLiPS population of the present invention is the Xuri Cell Expansion System available from GE Healthcare. The cultivation of the CLiPS population in an automated system such as the Quantum® Cell Expansion System is of particular benefit if a working cell bank for therapeutic application is to be produced under GMP conditions and a high number of cells is wanted. Also for the subcultivation, CLiPS can be cultured till a suitable amount of cells have grown. In illustrative examples CLiPS are subcultivated till the CLiPS reach about 70% to about 80% confluency. The isolation/cultivation of the population of CLiPS can be carried out under standard condition for the cultivation of mammalian cells. Once a desired/suitable number of CLiPS have been obtained from the subculture, the cells are harvested by removing them from the cultivation vessel used for the subcultivation. The CLiPS harvesting is typically carried out by enzymatic treatment. The isolated CLiPS are subsequently collected and are either be directly used or preserved for further use. Typically, preserving is carried out by cryo-preservation. The term “cryo-preservation” is used herein in its regular meaning to describe a process where here CLiPS are preserved by cooling to low sub-zero temperatures, such as (typically) −80° C. or −196° C. (the boiling point of liquid nitrogen). Cryopreservation can be carried out as known to the person skilled in the art and can include the use of cryo-protectors such as dimethylsulfoxide (DMSO) or glycerol, which slow down the formation of ice-crystals in the CLiPS cells.

The present invention is also directed to CLiPS obtainable by the method as described herein and to CLiPS obtained by the method as described herein. CLiPS obtainable/obtained by the present invention may grow and proliferate robustly (cf. Example 2 and Example 3). Thereby, CLiPS cultivation may be more efficient in comparison to a cultivation of iPS derived from, for example, the bone marrow stroma, fat tissue, the dermis or the Wharton's jelly. Analysis of CLiPS functionality reveals expression of human embryonic stem cell markers indicating self-renewal properties and a normal karyotype (cf. Example 4 and Example 5). Further, CLiPS are capable to differentiate into multiple cell types (functional target cells) in vitro and in vivo indicating pluripotency (cf. Example 6). Therefore, CLiPS are highly suitable for medical and therapeutic applications. Consequently, the present invention is also directed to a pharmaceutical composition comprising an iPS obtainable/obtained by the method described herein.

The present invention is further directed to a method of differentiating a CLiPS into a target cell under conditions suitable for differentiation. Examples of a suitable target cell include, but are by no means limited to, a neuronal cell, dopaminergic neuronal cell, an oligodentrocyte, an astrocyte, a cortical neuron, a hepatocyte, a cartilage cell, a muscle cell, a bone cell, a dental cell, a hair follicle cell, an inner ear hair cell, a skin cell, a melanocyte, a cardiomyocyte, a hematopoietic progenitor cell, a blood cell, an immune cell, a T- or B-lymphocyte, a microglia, a natural killer cell or a motor neuron, to mention only a few. To facilitate the directed differentiation into a target cell, the CLiPS may be exposed to a priming substance, typically under conditions that are known to the skilled artesian from the differentiation of iPS derived from other sources into the target cell. The exposure may be carried out under suitable conditions, which may comprise a cultivation in a cell culture vessel filled with a cell culture medium suitable for priming the CLiPS differentiation and for subsequent cultivation. In the present invention any cell culture medium suitable for priming, proliferating and differentiating iPS can be used, wherein the medium composition and thus the method of differentiation may depend on the target cell and may be taken from known protocols for the differentiation of iPS into the desired target cell (see in this respect, the reviews of Hirschi et al “Induced Pluripotent Stem Cells for Regenerative Medicine” Annu Rev Biomed Eng. 2014 Jul. 11; 16: 277-294) or Shi et al “Induced pluripotent stem cell technology: a decade of progress” Nat Rev Drug Discov. 2017 February; 16(2): 115-130). For example, CLiPS may be cultivated in a medium adapted for proliferation and differentiation of the CLiPS into a dopaminergic neuronal cell. In such a case, the medium may be a Neurobasal medium supplemented with a growth factor such as B-27 minus vitamin A, transforming growth factor 3-β (TGFβ3), a glial cell line-derived neurotrophic factor (GDNF), a brain-derived neurotrophic factor (BDNF), ascorbic acid, dibutyl cAMP, an inhibitor for glycogen synthase kinase 3 such as CHIR99021 and a γ-secretase inhibitor such as (2S)-N-[(3,5-Difluorophenyl)acetyl]-L-alanyl-2-phenyl]glycine 1,1-dimethylethyl ester (DAPT), which induces neuronal differentiation. An illustrative example for such a medium is NB27. A CLiPS differentiation into a dopaminergic neuronal cell is exemplary shown in Example 7. As another example, CLiPS may be cultivated in a medium adapted for proliferation and differentiation of the CLiPS into a hepatocyte. In this case, the medium may be a protein, lipid and growth factor-free medium supplemented with a compound inducing differentiation into a mesoendodermal fate. RPMI 1640-B27 supplemented with Activin A may be an illustrative example for a suitable medium for CLiPS differentiation into a hepatocyte. A CLiPS differentiation into a hepatocyte is exemplary shown in Example 8. As another illustrative example, CLiPS may be cultivated in a medium adapted for proliferation and differentiation of the CLiPS into a cardiomyocyte. In such a case, the medium may be a protein, lipid and growth factor-free medium supplemented with an inhibitor for glycogen synthase kinase 3 such as CHIR99021. RPMI/2%-B27 minus insulin may be an example for a suitable medium for CLiPS differentiation into a hepatocyte. A CLiPS differentiation into a cardiomyocyte is exemplary shown in Example 9. As yet a further illustrative example, CLiPS may be differentiated into an oligodendrocyte using a chemically defined, growth factor-rich medium allowing a differentiation into paired box 6-positive (PAX6+) neural stem cells, which then give rise to oligodendrocyte transcription factor positive (OLIG2+) progenitors (cf. Example 10). In this context, it may be noted that the differentiation of CLiPS into target cells may also be carried out under conditions suitable for cGMP production.

The present invention also includes a pharmaceutical composition comprising a differentiated CLiPS obtained by the method as described herein. The analysis of the immunogenicity of CLiPS and their neural derivates revealed reduced immunogenicity (Example 11). An example for a pharmaceutical composition comprising differentiated CLiPS is an injection solution or any kind of graft suitable for implanting the differentiated CLiPS. In one example, such a graft may comprise differentiated CLiPS-derived multilayered tissue such as an organ or parts thereof. In one example, the graft suitable for implanting the differentiated CLiPS may comprise an implantable matrix coated with differentiated CLiPS. The pharmaceutical composition may be formulated/adapted for parenteral application. In such case, the parenteral application may comprise a sterile preparation intended for injection, infusion or implantation in the human or animal body. Transplantation of CLiPS-derived dopaminergic neurons in fully immunocompetent mice and rat Parkinson's Disease models exhibited functional engraftment and even significant restoration of dopamine reuptake function (cf. Example 12 and Example 13).

The present invention further includes a method of treating a congenital or acquired degenerative disorder in a subject, wherein the subject may be selected from the group comprising a mouse, a rat, a rabbit, a pig, a dog, a cat, a non-human primate or a human. In a preferred example, the subject is human. In this context, treating may comprise administering to a subject a target cell differentiated from CLiPS by the method as described herein. The disease may any known disease which has been considered to be treated by means of cell-based therapy, see in this context, for example, Shi et al “Induced pluripotent stem cell technology: a decade of progress” supra. The congenital or acquired degenerative disorder may have different origins. For example, such a congenital or acquired degenerative disorder may be a neural disorder such as, for example, Parkinson's disease, Alzheimer's disease, Huntington's disease, Amyotrophic lateral sclerosis (ALS), Spinocerebellar ataxia (SCA) and batten disease. Examples for a hepatic degenerative disorder may be inter alia liver failure, liver cirrhosis and viral hepatitis. The congenital or acquired degenerative disorder may also be a cardiac disorder, inter alia comprising acute Danon disease, short-QT syndrome, Brugada syndrome, myocardial infarction, Jervell and Lange-Nielsen syndrome. The disorder may also be an auto-immune disease such as multiple sclerosis.

The present invention is also directed to extracellular membranous vesicles that may be produced by CLiPS or the differentiated derivatives of CLiPS. Such vesicles may include but not exclusively, vesicles ranging from 30 to 150 nanometres (nm) in diameter, also known as exosomes. Originally thought to be primarily responsible for excretory functions, exosomes are now known to be involved in various important biological processes such as cell-cell communication, cellular senescence, proliferation, and differentiation, tissue homeostasis, tissue repair and regeneration, antigen presentation and immune modulation (see, for example, Pegtel, D. M. and S. J. Gould, Exosomes. Annu Rev Biochem, 2019. 88: p. 487-514 or Kalluri, R. and V. S. LeBleu, The biology, function, and biomedical applications of exosomes. Science, 2020. 367(6478). Exosomes have been implicated in a broad range of diseases including cancers (see, for example, Visan, K. S., R. J. Lobb, and A. Moller, The role of exosomes in the promotion of epithelial-to-mesenchymal transition and metastasis. Front Biosci (Landmark Ed), 2020. 25: p. 1022-1057, or Zhang, L. and D. Yu, Exosomes in cancer development, metastasis, and immunity. Biochim Biophys Acta Rev Cancer, 2019. 1871(2): p. 455-468) osteoarthritis (Asghar, S., et al., Exosomes in intercellular communication and implications for osteoarthritis. Rheumatology (Oxford), 2020. 59(1): p. 57-68), diseases of the central nervous system such as such as stroke, Alzheimer's disease (AD), Parkinson's disease (PD), prion disease, and amyotrophic lateral sclerosis (ALS) (see, for example, Liu, W., et al., Role of Exosomes in Central Nervous System Diseases. Front Mol Neurosci, 2019. 12: p. 240 or Quek, C. and A. F. Hill, The role of extracellular vesicles in neurodegenerative diseases. Biochem Biophys Res Commun, 2017. 483(4): p. 1178-1186), mental disorders (Saeedi, S., et al., The emerging role of exosomes in mental disorders. Transl Psychiatry, 2019. 9(1): p. 122), cardiovascular diseases (Wang, Y., et al., Exosomes: An emerging factor in atherosclerosis. Biomed Pharmacother, 2019. 115: p. 108951), metabolic diseases (see, for example, Dini, L., et al., Microvesicles and exosomes in metabolic diseases and inflammation. Cytokine Growth Factor Rev, 2020. 51: p. 27-39 or Soazig, L. L., A. Ramaroson, and M. M. Carmen, Exosomes in metabolic syndrome, in Exosomes: A Clinical Compendium, L. R. Edelstein, et al., Editors. 2020, Academic Press. p. 343-356) and many more.

Exosome cargoes have been shown to consist of various biomolecules including proteins, lipids and nucleic acids. RNA species such as tRNA, mRNA, lncRNA, circular RNA and miRNA can potentially regulate gene expression in target cells and tissue. Exosomes produced by certain cell types have been shown to possess therapeutic properties. In this respect, mesenchymal stem cells (MSCs) isolated from different sources such as bone marrow, adipose tissue, and umbilical cord have emerged as particularly favourable. MSC-derived exosomes to have shown potential therapeutic effects in animal models of cornea, cardiovascular, Alzheimer's, Parkinson's and inflammatory bowel diseases, among others. In addition to endogenous cells, in vitro cultured pluripotent stem cells (PSCs) such as embryonic stem cells (ESCs) and induced pluripotent stem cells (iPS) have been shown to produce exosomes (Song, Y. H., et al., Exosomes Derived from Embryonic Stem Cells as Potential Treatment for Cardiovascular Diseases. Adv Exp Med Biol, 2017. 998: p. 187-206. or Jeske, R., et al., Human Pluripotent Stem Cell-Derived Extracellular Vesicles: Characteristics and Applications. Tissue Eng Part B Rev, 2020. 26(2): p. 129-144. Administration of cell-free iPS-derived exosomes is considered to be safer than iPS-derived cells due to the risk of tumour formation from residual undifferentiated cells (Riazifar, M., et al., Stem Cell Extracellular Vesicles: Extended Messages of Regeneration. Annu Rev Pharmacol Toxicol, 2017. 57: p. 125-154). Notably, therapeutic properties have also been demonstrated for exosomes isolated from differentiated derivatives of iPS. For example, treatment with exosomes purified from iPS-derived cardiomyocytes enhanced cardiac recovery in mouse model of myocardial infarction, with significant reduction in apoptosis and fibrosis compared to untreated animals. The exosomes also rescued in vitro cultures of iPS-cardiomyocytes from hypoxia and exosome biogenesis inhibition (Liu, B., et al., Cardiac recovery via extended cell-free delivery of extracellular vesicles secreted by cardiomyocytes derived from induced pluripotent stem cells. Nat Biomed Eng, 2018. 2(5): p. 293-303). In another study, exosomes from iPS-derived MSCs exosomes isolated from iPS-derived MSCs accelerated the proliferation of human dermal fibroblasts and human keratinocytes, and enhanced wound healing in in vitro scratch assays. There was no significant difference in the effects of these exosomes compared to those isolated from primary MSCs (Kim, S., et al., Exosomes Secreted from Induced Pluripotent Stem Cell-Derived Mesenchymal Stem Cells Accelerate Skin Cell Proliferation. Int J Mol Sci, 2018. 19(10).

Thus, in accordance with these reports, extracellular membranous vesicles or exosomes produced by CLiPS (either derived from CLMC or CLEC) or the differentiated derivatives of CLiPS of the present invention are considered useful for the treatment of diseases including the above-mentioned exemplary disease such as cancer, osteoarthritis, diseases of the central nervous system such as such as stroke, Alzheimer's disease (AD), Parkinson's disease (PD), prion disease, and amyotrophic lateral sclerosis (ALS), mental disorders or metabolic diseases.

In addition, taking advantage of their efficient cargo delivery ability, exosomes are actively pursued as delivery carriers for facilitating cellular uptake of various therapeutic agents such as microRNA, drugs, and peptides (see Antimisiaris, S. G., S. Mourtas, and A. Marazioti, Exosomes and Exosome-Inspired Vesicles for Targeted Drug Delivery. Pharmaceutics, 2018. 10(4), Liao, W., et al., Exosomes: The next generation of endogenous nanomaterials for advanced drug delivery and therapy. Acta Biomater, 2019. 86: p. 1-14 or Wang, X., et al., Cell-derived Exosomes as Promising Carriers for Drug Delivery and Targeted Therapy. Curr Cancer Drug Targets, 2018. 18(4): p. 347-354. In line with this, extracellular membranous vesicles or exosomes produced by CLiPS (either derived from CLMC or CLEC) or the differentiated derivatives of CLiPS of the present invention are considered can also be uses as delivery carriers for facilitating cellular uptake of therapeutic agents. Accordingly, the invention also encompasses the use of CLiPS or the differentiated derivatives of CLiPS for the purpose of delivery of exogenously loaded or transgenically expressed molecules.

Extracellular membranous vesicles and exosomes produced by CLiPS (either derived from CLMC or CLEC) or the differentiated derivatives of CLiPS can be isolated using respective methods described in the literature. Typically, exosomes are purified from the extracellular milieu into which they are secreted. Known methods for the isolation of exosomes include ultracentrifugation, ultrafiltration, size-exclusion chromatography, field-flow fractionation, polymer coprecipitation, immunoaffinity, microfluidics, or acoustic nanofilter. All these methods can be used for the isolation of exosomes produced by CLiPS or the differentiated derivatives of CLiPS described here.

The present invention further relates a particular method of differentiating an iPS cell, which is derived from a stem cell of the amniotic membrane of the umbilical cord as defined elsewhere herein and which refers to CLiPS as also defined herein, into a RPE cell. Said differentiation method comprises culturing said CLiPs in a differentiation medium under conditions suitable for the differentiation into a RPE cell.

A “retinal pigment epithelial (short: RPE) cell” refers to a cell derived from/from/taken from the retinal pigment epithelium. In other words, such cell is comprised by said retinal pigment epithelium and will be defined in more detail below. RPE differentiation from CLiPs was achieved using the rapid, directed and modified differentiation method according to the present invention. The CLiPS as used herein for differentiating into a RPE cell may be derived from umbilical cord-lining mesenchymal cells (such as CLMC23, CLMC30, CLMC44) and/or from umbilical cord-lining ectodermal cells (such as CLEC23). In a preferred embodiment, the CLiPS as used herein for differentiating into a RPE cell is any one of CLMC23, CLMC30, CLMC44 or CLEC23. In a preferred embodiment, the CLiPS as used herein for differentiating into a RPE cell is CLMC23. In another preferred embodiment, the CLiPS as used herein for differentiating into a RPE cell is CLMC30. In another preferred embodiment, the CLiPS as used herein for differentiating into a RPE cell is CLMC44. In another preferred embodiment, the CLiPS as used herein for differentiating into a RPE cell is CLEC23. A RPE cell differentiated from a CLiPS as defined herein by the differentiation method as described herein may refer to a CLiPS-derived RPE cell or to a CLiPS-RPE. The differentiation of a RPE cell derived from CLiPs may be compared to the differentiation of a RPE cell derived from a ES cell such as H9 ES cell (also called ES derived RPE when referring to such RPE cell) and/or to the differentiation of a RPE cell derived from an iPS cell derived from the skin (also called skin iPS; thus skin iPS derived RPE when referring to such RPE cell) such as Asf5, AGO, or HDFA cell using the differentitation method according to the present invention (see the Example section).

The differentiation medium as used in the differentiation method of differentiating an iPS cell into a RPE cell comprising culturing iPS cells derived from a stem cell of the amniotic membrane of the umbilical cord is preferably a DMEM (Dulbecco's modified eagle medium) medium as defined herein comprising N2 supplement, B27 supplement and non-essential amino acid (NEAA), even more preferably a DMEM (Dulbecco's modified eagle medium)/F12 (Ham's F12 medium) medium as defined elsewhere herein comprising N2 supplement, B27 supplement and non-essential amino acid (NEAA). In a preferred embodiment, the DMEM/F12 medium used in the differentiation method of differentiating an iPS cell into a RPE cell comprises 1× N2 supplement, 1× B27 supplement, and 1× NEAA. Comprising 1× N2 supplement, 1× B27 supplement and 1× NEAA in said medium means that the final concentration is 1× as can also be seen in the embodiment below. In said embodiment, the differentiation medium, preferably DMEM medium, even more preferably DMEM/F12 medium as defined herein, is obtained by mixing to obtain a final volume of 1000 ml culture medium:
10 ml of 100× N2 supplement;
20 mL of 50× B27 supplement;
10 mL of 100× NEAA;
960 mL of DMEM, preferably of DMEM/F12.

The differentiation medium as defined herein may further comprise/being supplemented with various growth factors and/or cytokines as defined elsewhere herein. Such differentiation medium as defined above may refer to a base medium for iPS culturing. Such base differentiation medium may then further be modified/supplemented for culturing iPS cells as defined herein in order for said cells to differentiate into RPE cells by using the method of differentiating an iPS cell into a RPE cell according to the present invention.

Particularly, the differentiation medium used in the method of differentiating an iPS cell into a RPE cell according to the present invention for culturing iPS cells so that said cells differentiate into RPE cells, may comprise a first differentiation medium additionally comprising at least any one of IGF1, DKK1, nicotinamide or LDN-193189. Said first differentiation medium is based on said base medium comprising DMEM medium, preferably DMEM/F12 medium comprising N2 supplement, B27 supplement and NEAA, even more preferably DMEM/F12 medium comprising 1× N2 supplement, 1× B27 supplement and 1× NEAA. In a preferred embodiment, the first differentiation medium as defined herein additionally comprises IGF1, DKK1, nicotinamide and LDN-193189.

Further, the differentiation medium used in the method of differentiating an iPS cell into a RPE cell according to the present invention for culturing iPS cells so that said cells differentiate into RPE cells, may additionally or alternatively comprise a second differentiation medium additionally comprising at least any one of IGF1, DKK1, nicotinamide, LDN-193189 or b-FGF. Said second differentiation medium is also based on said base medium comprising DMEM medium, preferably DMEM/F12 medium comprising N2 supplement, B27 supplement and NEAA, even more preferably DMEM/F12 medium comprising 1× N2 supplement, 1× B27 supplement and 1× NEAA. In a preferred embodiment, the second differentiation medium as defined herein additionally comprises IGF1, DKK1, nicotinamide, LDN-193189 and b-FGF.

Further, the differentiation medium used in the method of differentiating an iPS cell into a RPE cell according to the present invention for culturing iPS cells that said cells differentiate into RPE cells, may additionally or alternatively comprise a third differentiation medium additionally comprising at least any one of IGF1, DKK1, or Activin A. Said third differentiation medium is also based on said base medium comprising DMEM medium, preferably DMEM/F12 medium comprising N2 supplement, B27 supplement and NEAA, even more preferably DMEM/F12 medium comprising 1× N2 supplement, 1× B27 supplement and 1× NEAA. In a preferred embodiment, the third differentiation medium as defined herein additionally comprises IGF1, DKK1 and Activin A.

Further, the differentiation medium used in the method of differentiating an iPS cell into a RPE cell according to the present invention for culturing iPS cells that said cells differentiate into RPE cells, may additionally or alternatively comprise a fourth differentiation medium additionally comprising Activin A and SU5402 or Activin A and PD17307. Said fourth differentiation medium is also based on said base medium comprising DMEM medium, preferably DMEM/F12 medium comprising N2 supplement, B27 supplement and NEAA, even more preferably DMEM/F12 medium comprising 1× N2 supplement, 1× B27 supplement and 1× NEAA. In a preferred embodiment, the fourth differentiation medium as defined herein additionally comprises Activin A and PD17307. Since PD17307 is applied in lower concentrations compared to the fibroblast growth factor inhibitor SU5402 which reduces undesirable changes in gene expression caused by the application of a higher concentration when SU5402 is used, PD17307 is preferred by the differentiation method of the present invention (FIG. 19).

Further, the differentiation medium used in the method of differentiating an iPS cell into a RPE cell according to the present invention for culturing iPS cells that said cells differentiate into RPE cells, may additionally or alternatively comprise a fifth differentiation medium additionally comprising at least any one of Activin A, CHIR99021 or SU5402; or comprising at least any one of Activin A, CHIR99021 or PD17307. Said fifth differentiation medium is also based on said base medium comprising DMEM medium, preferably DMEM/F12 medium comprising N2 supplement, B27 supplement and NEAA, even more preferably DMEM/F12 medium comprising 1× N2 supplement, 1× B27 supplement and 1× NEAA. In a preferred embodiment, the fifth differentiation medium as defined herein additionally comprises Activin A, CHIR99021 and PD17307 (using PD17307 for the same reasons as defined above). The fifth differentiation medium preferably comprises Activin A, SU5402 or PD17307, preferably PD17307, and a first concentration of CHIR99021, which is below 3 μM. The concentration of CHIR99021, an activator of Wnt signalling pathway, is then increased, when the fifth differentiation medium is again used to culture iPS cells to differentiate into RPE cells. The gradual increase of the concentration prevents excessive cell death caused by the high concentration of CHIR when used. This improved the yield of pigmented RPE cells. When the fifth differentiation medium is then subsequently applied it preferably comprises Activin A, SU5402 or PD17307, preferably PD17307, and a second concentration of CHIR99021, which is about 3 μM.

When IGF1 as supplement is applied in the differentiation medium as defined elsewhere herein, a final concentration of at least about 5 ng/ml, at least about 6 ng/ml, at least about 7 ng/ml, at least about 8 ng/ml, at least about 9 ng/ml, or at least about 10 ng/ml; or within a range of about 5 to about 15 ng/ml, of about 6 to about 14 ng/ml, of about 7 to about 13 ng/ml, of about 8 to about 12 ng/ml, of about 9 to about 11 ng/ml is used. In a preferred embodiment, IGF1 is used in the differentiation medium as defined elsewhere herein in a final concentration of about 10 ng/ml. In an even more preferred embodiment IGF1 is used in the first differentiation medium as defined elsewhere herein in a final concentration of at least about 5 ng/ml, at least about 6 ng/ml, at least about 7 ng/ml, at least about 8 ng/ml, at least about 9 ng/ml, or at least about 10 ng/ml; or within a range of about 5 to about 15 ng/ml, of about 6 to about 14 ng/ml, of about 7 to about 13 ng/ml, of about 8 to about 12 ng/ml, of about 9 to about 11 ng/ml, most preferably in a final concentration of about 10 ng/ml. In another even more preferred embodiment, IGF1 is used in the second differentiation medium as defined elsewhere herein in a final concentration of at least about 5 ng/ml, at least about 6 ng/ml, at least about 7 ng/ml, at least about 8 ng/ml, at least about 9 ng/ml, or at least about 10 ng/ml; or within a range of about 5 to about 15 ng/ml, of about 6 to about 14 ng/ml, of about 7 to about 13 ng/ml, of about 8 to about 12 ng/ml, of about 9 to about 11 ng/ml, most preferably in a final concentration of about 10 ng/ml. In another even more preferred embodiment, IGF1 is used in the third differentiation medium as defined elsewhere herein in a final concentration of at least about 5 ng/ml, at least about 6 ng/ml, at least about 7 ng/ml, at least about 8 ng/ml, at least about 9 ng/ml, or at least about 10 ng/ml; or within a range of about 5 to about 15 ng/ml, of about 6 to about 14 ng/ml, of about 7 to about 13 ng/ml, of about 8 to about 12 ng/ml, of about 9 to about 11 ng/ml, most preferably in a final concentration of about 10 ng/ml. The present invention further comprises the method of differentiating into a RPE cell, the method comprising culturing said iPS cell in a differentiation medium as defined elsewhere herein, wherein IGF1 is applied in said differentiation medium as defined herein for at least about 2 days, at least about 3 days, at least about 4 days, at least about 5 days, at least about 6 days, preferably for about 6 days, even more preferably for about 6 consecutive days, most preferably used for about 6 (consecutive) days in a final concentration of about 10 ng/ml. When IGF1 is applied in the first differentiation medium as defined herein, IGF1 is used for about 2 days, preferably for about 2 consecutive days, meaning on day 0 to day 2 of the culture, even more preferably used for about 2 (consecutive) days in a final concentration of about 10 ng/ml. When IGF1 is applied in the second differentiation medium as defined herein, IGF1 is used for about 2 days, preferably for about 2 consecutive days, meaning on day 2 to day 4 of the culture, even more preferably used for about 2 (consecutive) days in a final concentration of about 10 ng/ml. When IGF1 is applied in the third differentiation medium as defined herein, IGF1 is used for about 2 days, preferably for about 2 consecutive days, meaning on day 4 to day 6 of the culture, even more preferably used for about 2 (consecutive) days in a final concentration of about 10 ng/ml.

When DKK1 is applied in the differentiation medium as defined elsewhere herein, a final concentration of at least about 5 ng/ml, at least about 6 ng/ml, at least about 7 ng/ml, at least about 8 ng/ml, at least about 9 ng/ml, or at least about 10 ng/ml; or within a range of about 5 to about 15 ng/ml, of about 6 to about 14 ng/ml, of about 7 to about 13 ng/ml, of about 8 to about 12 ng/ml, of about 9 to about 11 ng/ml is used. In a preferred embodiment, DKK1 is used in the differentiation medium as defined elsewhere herein in a final concentration of about 10 ng/ml. In an even more preferred embodiment DKK1 is used in the first differentiation medium as defined elsewhere herein in a final concentration of at least about 5 ng/ml, at least about 6 ng/ml, at least about 7 ng/ml, at least about 8 ng/ml, at least about 9 ng/ml, or at least about 10 ng/ml; or within a range of about 5 to about 15 ng/ml, of about 6 to about 14 ng/ml, of about 7 to about 13 ng/ml, of about 8 to about 12 ng/ml, of about 9 to about 11 ng/ml, most preferably in a final concentration of about 10 ng/ml. In another even more preferred embodiment DKK1 is used in the second differentiation medium as defined elsewhere herein in a final concentration of at least about 5 ng/ml, at least about 6 ng/ml, at least about 7 ng/ml, at least about 8 ng/ml, at least about 9 ng/ml, or at least about 10 ng/ml; or within a range of about 5 to about 15 ng/ml, of about 6 to about 14 ng/ml, of about 7 to about 13 ng/ml, of about 8 to about 12 ng/ml, of about 9 to about 11 ng/ml, most preferably in a final concentration of about 10 ng/ml. In another even more preferred embodiment DKK1 is used in the third differentiation medium as defined elsewhere herein in a final concentration of at least about 5 ng/ml, at least about 6 ng/ml, at least about 7 ng/ml, at least about 8 ng/ml, at least about 9 ng/ml, or at least about 10 ng/ml; or within a range of about 5 to about 15 ng/ml, of about 6 to about 14 ng/ml, of about 7 to about 13 ng/ml, of about 8 to about 12 ng/ml, of about 9 to about 11 ng/ml, most preferably in a final concentration of about 10 ng/ml. The present invention further comprises the method of differentiating into a RPE cell, the method comprising culturing said iPS cell in a differentiation medium as defined elsewhere herein, wherein DKK1 is applied in said differentiation medium as defined herein for at least about 2 days, at least about 3 days, at least about 4 days, at least about 5 days, at least about 6 days, preferably for about 6 days, even more preferably for about 6 consecutive days, most preferably used for about 6 (consecutive) days in a final concentration of about 10 ng/ml. When DKK1 is applied in the first differentiation medium as defined herein, DKK1 is used for about 2 days, preferably for about 2 consecutive days, meaning on day 0 to day 2 of the culture, even more preferably used for about 2 (consecutive) days in a final concentration of about 10 ng/ml. When DKK1 is applied in the second differentiation medium as defined herein, DKK1 is used for about 2 days, preferably for about 2 consecutive days, meaning on day 2 to day 4 of the culture, even more preferably used for about 2 (consecutive) days in a final concentration of about 10 ng/ml. When DKK1 is applied in the third differentiation medium as defined herein, DKK1 is used for about 2 days, preferably for about 2 consecutive days, meaning on day 4 to day 6 of the culture, even more preferably used for about 2 (consecutive) days in a final concentration of about 10 ng/ml.

When nicotinamide is applied in the differentiation medium as defined elsewhere herein, a final concentration of at least about 5 mM, at least about 6 mM, at least about 7 mM, at least about 8 mM, at least about 9 mM, or at least about 10 mM; or within a range of about 5 to about 15 mM, of about 6 to about 14 mM, of about 7 to about 13 mM, of about 8 to about 12 mM, of about 9 to about 11 mM is used. In a preferred embodiment, nicotinamide is used in the differentiation medium as defined elsewhere herein in a final concentration of about 10 mM. In an even more preferred embodiment nicotinamide is used in the first differentiation medium as defined elsewhere herein in a final concentration of at least about 5 mM, at least about 6 mM, at least about 7 mM, at least about 8 mM, at least about 9 mM, or at least about 10 mM; or within a range of about 5 to about 15 mM, of about 6 to about 14 mM, of about 7 to about 13 mM, of about 8 to about 12 mM, of about 9 to about 11 mM, most preferably in a final concentration of about 10 mM. In another even more preferred embodiment, nicotinamide is used in the second differentiation medium as defined elsewhere herein in a final concentration of at least about 5 mM, at least about 6 mM, at least about 7 mM, at least about 8 mM, at least about 9 mM, or at least about 10 mM; or within a range of about 5 to about 15 mM, of about 6 to about 14 mM, of about 7 to about 13 mM, of about 8 to about 12 mM, of about 9 to about 11 mM, most preferably in a final concentration of about 10 mM. The present invention further comprises the method of differentiating into a RPE cell, the method comprising culturing said iPS cell in a differentiation medium as defined elsewhere herein, wherein nicotinamide is applied in said differentiation medium as defined herein for at least about 2 days, at least about 3 days, at least about 4 days, preferably for about 4 days, even more preferably for about 4 consecutive days, most preferably used for about 4 (consecutive) days in a final concentration of about 10 mM. When nicotinamide is applied in the first differentiation medium as defined herein, nicotinamide is used for about 2 days, preferably for about 2 consecutive days, meaning on day 0 to day 2 of the culture, even more preferably used for about 2 (consecutive) days in a final concentration of about 10 mM. When nicotinamide is applied in the second differentiation medium as defined herein, nicotinamide is used for about 2 days, preferably for about 2 consecutive days, meaning on day 2 to day 4 of the culture, even more preferably used for about 2 (consecutive) days in a final concentration of about 10 mM.

When LDN-193189 is applied in the differentiation medium as defined elsewhere herein, a final concentration of at least about 0.1 μM, at least about 0.2 μM, at least about 0.3 μM, at least about 0.4 μM, at least about 0.5 μM, at least about 0.6 μM, at least about 0.7 μM, at least about 0.8 μM, at least about 0.9 μM, or at least about 1 μM is used. In a preferred embodiment, LDN-193189 is used in the differentiation medium as defined elsewhere herein in a final concentration of about 1 μM. In another preferred embodiment, LDN-193189 is used in the differentiation medium as defined elsewhere herein in a final concentration of about 0.2 μM. In an even more preferred embodiment, LDN-193189 is used in the first differentiation medium as defined elsewhere herein in a final concentration within a range of about 0.5 to about 1.5 μM, of about 0.6 to about 1.4 μM, of about 0.7 to about 1.3 μM, of about 0.8 to about 1.2 μM, of about 0.9 to about 1.1 μM, most preferably in a final concentration of about 1 μM. In another even more preferred embodiment, LDN-193189 is used in the second differentiation medium as defined elsewhere herein in a final concentration within a range of about 0.1 to about 0.3 μM, of about 0.11 to about 0.29 μM, of about 0.12 to about 0.28 μM, of about 0.13 to about 0.27 μM, of about 0.14 to about 0.26 μM, of about 0.15 to about 0.25 μM, most preferably in a final concentration of about 0.2 μM. The present invention further comprises the method of differentiating into a RPE cell, the method comprising culturing said iPS cell in a differentiation medium as defined elsewhere herein, wherein LDN-193189 is applied in said differentiation medium as defined herein for at least about 2 days, at least about 3 days, at least about 4 days, preferably for about 4 days, even more preferably for about 4 consecutive days, most preferably used for about 4 (consecutive) days in a concentration of at least about 0.1 μM as defined elsewhere herein. When LDN-193189 is applied in the first differentiation medium as defined herein, LDN-193189 is used for about 2 days, preferably for about 2 consecutive days, meaning on day 0 to day 2 of the culture, even more preferably LDN-193189 is used for about 2 (consecutive) days in a concentration of about 1 μM. When LDN-193189 is applied in the second differentiation medium as defined herein, LDN-193189 is used for about 2 days, preferably for about 2 consecutive days, meaning on day 2 to day 4 of the culture, even more preferably LDN-193189 is used for about 2 (consecutive) days in a concentration of about 0.2 μM.

When b-FGF is applied in the differentiation medium as defined elsewhere herein a final concentration of at least about 2.5 ng/ml, at least about 3 ng/ml, at least about 3.5 ng/ml, at least about 4 ng/ml, at least about 4.5 ng/ml, or at least about 5 ng/ml; or within a range of about 2.5 to about 7.5 ng/ml, of about 3 to about 7 ng/ml, of about 3.5 to about 6.5 ng/ml, of about 4 to about 6 ng/ml, of about 4.5 to about 5.5 ng/ml is used. In a preferred embodiment, b-FGF is used in the differentiation medium as defined elsewhere herein in a final concentration of about 5 ng/ml. In an even more preferred embodiment, b-FGF is used in the second differentiation medium as defined elsewhere herein in a final concentration of at least about 2.5 ng/ml, at least about 3 ng/ml, at least about 3.5 ng/ml, at least about 4 ng/ml, at least about 4.5 ng/ml, or at least about 5 ng/ml; or within a range of about 2.5 to about 7.5 ng/ml, of about 3 to about 7 ng/ml, of about 3.5 to about 6.5 ng/ml, of about 4 to about 6 ng/ml, of about 4.5 to about 5.5 ng/ml, most preferably in a final concentration of about 5 ng/ml. The present invention further comprises the method of differentiating into a RPE cell, the method comprising culturing said iPS cell in a differentiation medium as defined elsewhere herein, wherein b-FGF is applied in said differentiation medium as defined herein for at least about 1 day, at least about 2 days, preferably for about 2 days, even more preferably for about 2 consecutive days, most preferably used for about 2 (consecutive) days in a final concentration of about 5 ng/ml. When b-FGF is applied in the second differentiation medium as defined herein, b-FGF is used for about 2 days, preferably for about 2 consecutive days, meaning on day 2 to day 4 of the culture, even more preferably used for about 2 (consecutive) days in a final concentration of about 5 ng/ml.

When Activin A is applied in the differentiation medium as defined elsewhere herein, a final concentration of at least about 50 ng/ml, at least about 60 ng/ml, at least about 70 ng/ml, at least about 80 ng/ml, at least about 90 ng/ml, or at least about 100 ng/ml; or within a range of about 50 to about 150 ng/ml, of about 60 to about 140 ng/ml, of about 70 to about 130 ng/ml, of about 80 to about 120 ng/ml, of about 90 to about 110 ng/ml is used. In a preferred embodiment, Activin A is used in the differentiation medium as defined elsewhere herein in a final concentration of about 100 ng/ml. In an even more preferred embodiment Activin A is used in the third differentiation medium as defined elsewhere herein in a final concentration of at least about 50 ng/ml, at least about 60 ng/ml, at least about 70 ng/ml, at least about 80 ng/ml, at least about 90 ng/ml, or at least about 100 ng/ml; or within a range of about 50 to about 150 ng/ml, of about 60 to about 140 ng/ml, of about 70 to about 130 ng/ml, of about 80 to about 120 ng/ml, of about 90 to about 110 ng/ml, most preferably in a final concentration of about 100 ng/ml. In another even more preferred embodiment, Activin A is used in the fourth differentiation medium as defined elsewhere herein in a final concentration of at least about 50 ng/ml, at least about 60 ng/ml, at least about 70 ng/ml, at least about 80 ng/ml, at least about 90 ng/ml, or at least about 100 ng/ml; or within a range of about 50 to about 150 ng/ml, of about 60 to about 140 ng/ml, of about 70 to about 130 ng/ml, of about 80 to about 120 ng/ml, of about 90 to about 110 ng/ml, most preferably in a final concentration of about 100 ng/ml. In another even more preferred embodiment, Activin A is used in the fifth differentiation medium as defined elsewhere herein in a final concentration of at least about 50 ng/ml, at least about 60 ng/ml, at least about 70 ng/ml, at least about 80 ng/ml, at least about 90 ng/ml, or at least about 100 ng/ml; or within a range of about 50 to about 150 ng/ml, of about 60 to about 140 ng/ml, of about 70 to about 130 ng/ml, of about 80 to about 120 ng/ml, of about 90 to about 110 ng/ml, most preferably in a final concentration of about 100 ng/ml. The present invention further comprises the method of differentiating into a RPE cell, the method comprising culturing said iPS cell in a differentiation medium as defined elsewhere herein, wherein Activin A is applied in said differentiation medium as defined herein for at least about 2 days, at least about 3 days, at least about 4 days, at least about 5 days, at least about 6 days, at least about 8 days, at least about 10 days, at least about 12 days, preferably for about 12 days, even more preferably for about 12 consecutive days, most preferably used for about 12 (consecutive) days in a final concentration of about 100 ng/ml. When Activin A is applied in the third differentiation medium as defined herein, Activin A is used for about 2 days, preferably for about 2 consecutive days, meaning on day 4 to day 6 of the culture, even more preferably used for about 2 (consecutive) days in a final concentration of about 100 ng/ml. When Activin A is applied in the fourth differentiation medium as defined herein, Activin A is used for about 2 days, preferably for about 2 consecutive days, meaning on day 6 to day 8 of the culture, even more preferably used for about 2 (consecutive) days in a final concentration of about 100 ng/ml. When Activin A is applied in the fifth differentiation medium as defined herein, Activin A is used for about 8 days, preferably for about 8 consecutive days, meaning on day 8 to day 16 of the culture, even more preferably used for about 8 (consecutive) days in a final concentration of about 100 ng/ml.

When SU5402 is applied in the differentiation medium as defined elsewhere herein, a final concentration of at least about 5 μM, at least about 6 μM, at least about 7 μM, at least about 8 μM, at least about 9 μM, or at least about 10 μM; or within a range of about 5 to about 15 μM, of about 6 to about 14 μM, of about 7 to about 13 μM, of about 8 to about 12 μM, of about 9 to about 11 μM is used. In a preferred embodiment, SU5402 is used in the differentiation medium as defined elsewhere herein in a final concentration of about 10 μM. In an even more preferred embodiment, SU5402 is used in the fourth differentiation medium as defined elsewhere herein in a final concentration of at least about 5 μM, at least about 6 μM, at least about 7 μM, at least about 8 μM, at least about 9 μM, or at least about 10 μM; or within a range of about 5 to about 15 μM, of about 6 to about 14 μM, of about 7 to about 13 μM, of about 8 to about 12 μM, of about 9 to about 11 μM, most preferably in a final concentration of about 10 μM. In another even more preferred embodiment, SU5402 is used in the fifth differentiation medium as defined elsewhere herein in a final concentration of at least about 5 μM, at least about 6 μM, at least about 7 μM, at least about 8 μM, at least about 9 μM, or at least about 10 μM; or within a range of about 5 to about 15 μM, of about 6 to about 14 μM, of about 7 to about 13 μM, of about 8 to about 12 μM, of about 9 to about 11 μM, most preferably in a final concentration of about 10 μM. The present invention further comprises the method of differentiating into a RPE cell, the method comprising culturing said iPS cell in a differentiation medium as defined elsewhere herein, wherein SU5402 is applied in said differentiation medium as defined herein for about 10 days, even more preferably for about 10 consecutive days, most preferably used for about 10 (consecutive) days in a concentration of about 10 μM as defined elsewhere herein. When SU5402 is applied in the fourth differentiation medium as defined herein, SU5402 is used for about 2 days, preferably for about 2 consecutive days, meaning on day 6 to day 8 of the culture, even more preferably SU5402 is used for about 2 (consecutive) days in a concentration of about 10 μM. When SU5402 is applied in the fifth differentiation medium as defined herein, SU5402 is used for about 8 days, preferably for about 8 consecutive days, meaning on day 8 to day 16 of the culture, even more preferably used for about 8 (consecutive) days in a final concentration of about 10 μM.

When PD17307 is applied in the differentiation medium as defined elsewhere herein, a final concentration of at least about 0.5 μM, at least about 0.6 μM, at least about 0.7 μM, at least about 0.8 μM, at least about 0.9 μM, or at least about 1 μM; or within a range of about 0.5 to about 1.5 μM, of about 0.6 to about 1.4 μM, of about 0.7 to about 1.3 μM, of about 0.8 to about 1.2 μM, of about 0.9 to about 1.1 μM is used. In a preferred embodiment, PD17307 is used in the differentiation medium as defined elsewhere herein in a final concentration of about 1 μM. In an even more preferred embodiment, PD17307 is used in the fourth differentiation medium as defined elsewhere herein in a final concentration of at least about 0.5 μM, at least about 0.6 μM, at least about 0.7 μM, at least about 0.8 μM, at least about 0.9 μM, or at least about 1 μM; or within a range of about 0.5 to about 1.5 μM, of about 0.6 to about 1.4 μM, of about 0.7 to about 1.3 μM, of about 0.8 to about 1.2 μM, of about 0.9 to about 1.1, most preferably in a final concentration of about 1 μM. In another even more preferred embodiment, PD17307 is used in the fifth differentiation medium as defined elsewhere herein in a final concentration of at least about 0.5 μM, at least about 0.6 μM, at least about 0.7 μM, at least about 0.8 μM, at least about 0.9 μM, or at least about 1 μM; or within a range of about 0.5 to about 1.5 μM, of about 0.6 to about 1.4 μM, of about 0.7 to about 1.3 μM, of about 0.8 to about 1.2 μM, of about 0.9 to about 1.1, most preferably in a final concentration of about 1 μM. The present invention further comprises the method of differentiating into a RPE cell, the method comprising culturing said iPS cell in a differentiation medium as defined elsewhere herein, wherein PD17307 is applied in said differentiation medium as defined herein for about 10 days, even more preferably for about 10 consecutive days, most preferably used for about 10 (consecutive) days in a concentration of about 1 μM as defined elsewhere herein. When PD17307 is applied in the fourth differentiation medium as defined herein, PD17307 is used for about 2 days, preferably for about 2 consecutive days, meaning on day 6 to day 8 of the culture, even more preferably PD17307 is used for about 2 (consecutive) days in a concentration of about 1 μM. When PD17307 is applied in the fifth differentiation medium as defined herein, PD17307 is used for about 8 days, preferably for about 8 consecutive days, meaning on day 8 to day 16 of the culture, even more preferably used for about 8 (consecutive) days in a final concentration of about 1 μM.

When CHIR99021 is applied in the differentiation medium as defined elsewhere herein, a final concentration of at least about 1 μM and less than about 3 μM, of at least about 1.1 μM and less than about 3 μM, of at least about 1.2 μM and less than about 3 μM, of at least about 1.3 μM and less than about 3 μM, of at least about 1.4 μM and less than about 3 μM, of at least about 1 μM and less than about 2.5 μM, of at least about 1 μM and less than about 2 μM, of at least about 1 μM and less than about 1.9 μM, of at least about 1 μM and less than about 1.8 μM, of at least about 1 μM and less than about 1.7 μM, of at least about 1 μM and less than about 1.6 μM is used. In a preferred embodiment, CHIR99021 is used in the differentiation medium as defined elsewhere herein in a final concentration of about 1.5 μM. CHIR99021 as used in the differentiation medium as defined herein may be applied for culturing the cell, namely the iPS cell of the invention which differentiates or has already been differentiated into a RPE cell, for about 3 consecutive culture days. In a more preferred embodiment, CHIR99021 is used in the differentiation medium as defined elsewhere herein in a final concentration of about 1.5 μM for culturing the cell, namely the iPS cell of the invention which differentiates or has already been differentiated into a RPE cell, for about 3 consecutive culture days.

In an even more preferred embodiment, CHIR99021 is used in the fifth differentiation medium as defined elsewhere herein in a final concentration of at least about 1 μM and less than about 3 μM, of at least about 1.1 μM and less than about 3 μM, of at least about 1.2 μM and less than about 3 μM, of at least about 1.3 μM and less than about 3 μM, of at least about 1.4 μM and less than about 3 μM, of at least about 1 μM and less than about 2.5 μM, of at least about 1 μM and less than about 2 μM, of at least about 1 μM and less than about 1.9 μM, of at least about 1 μM and less than about 1.8 μM, of at least about 1 μM and less than about 1.7 μM, of at least about 1 μM and less than about 1.6 μM, most preferably in a final concentration of about 1.5 μM. In an even more preferred embodiment, CHIR99021 is used in the fifth differentiation medium as defined elsewhere herein in a final concentration of at least about 1 μM and less than about 3 μM, of at least about 1.1 μM and less than about 3 μM, of at least about 1.2 μM and less than about 3 μM, of at least about 1.3 μM and less than about 3 μM, of at least about 1.4 μM and less than about 3 μM, of at least about 1 μM and less than about 2.5 μM, of at least about 1 μM and less than about 2 μM, of at least about 1 μM and less than about 1.9 μM, of at least about 1 μM and less than about 1.8 μM, of at least about 1 μM and less than about 1.7 μM, of at least about 1 μM and less than about 1.6 μM for culturing the cell, namely the iPS cell of the invention which differentiates or has already been differentiated into a RPE cell, for about 3 consecutive culture days, most preferably in a final concentration of about 1.5 μM for culturing the cell, namely the iPS cell of the invention which differentiates or has already been differentiated into a RPE cell, for about 3 consecutive culture days (meaning on day 8 to day 11 of the culture).

When CHIR99021 is applied to the differentiation medium, preferably to the fifth differentiation medium, as defined above, CHIR99021 is again applied in the differentiation medium for subsequently culturing the cell, namely the iPS cell of the invention which differentiates or has already been differentiated into a RPE cell, preferably subsequently culturing the cell, namely the iPS cell of the invention which differentiates or has already been differentiated into a RPE cell, for about 5 consecutive culture days. In a preferred embodiment, CHIR99021 is then used in a final concentration of about 3 μM for subsequently culturing the cell, namely the iPS cell of the invention which differentiates or has already been differentiated into a RPE cell, even more preferably subsequently culturing the cell, namely the iPS cell of the invention which differentiates or has already been differentiated into a RPE cell, for about 5 consecutive culture days (meaning on day 11 to day 16 of the culture). By gradually increasing the final concentration of 1.5 μM for 3 days followed by 3 μM for 5 days, the yield of pigmented RPE cells improves.

The present invention further comprises the method of differentiating into a RPE cell, the method comprising culturing said iPS cell in a differentiation medium as defined elsewhere herein, wherein CHIR99021 is used for about 8 culture days, preferably for about 8 consecutive culture days, even more preferably wherein during the first 3 (consecutive) days of the about 8 days of culturing with CHIR99021, CHIR99021 is used in a final concentration of at least about 1 μM and less than about 3 μM, of at least about 1.1 μM and less than about 3 μM, of at least about 1.2 μM and less than about 3 μM, of at least about 1.3 μM and less than about 3 μM, of at least about 1.4 μM and less than about 3 μM, of at least about 1 μM and less than about 2.5 μM, of at least about 1 μM and less than about 2 μM, of at least about 1 μM and less than about 1.9 μM, of at least about 1 μM and less than about 1.8 μM, of at least about 1 μM and less than about 1.7 μM, of at least about 1 μM and less than about 1.6 μM, most preferably in a final concentration of about 1.5 μM and wherein during the next 5 (consecutive) days of the about 8 days of culturing with CHIR99021, CHIR99021 is used in a final concentration of about 3 μM.

In a most preferred embodiment, the differentiation medium comprises a first differentiation medium comprising about 1 μM LDN-193189, about 10 ng/ml DKK1, about 10 ng/ml IGF1 and about 10 mM nicotinamide. In another most preferred embodiment, the differentiation medium comprises a second differentiation medium comprising about 0.2 μM LDN-193189, about 10 ng/ml DKK1, about 10 ng/ml IGF1, about 10 mM nicotinamide and about 5 ng/ml b-FGF. In another most preferred embodiment, the differentiation medium comprises a third differentiation medium comprising about 10 ng/ml DKK1, about 10 ng/ml IGF1 and about 100 ng/ml Activin A. In another most preferred embodiment, the differentiation medium comprises a fourth differentiation medium comprising about 100 ng/ml Activin A and about 10 μM SU5402, preferably comprising about 100 ng/ml Activin A and about 1 μM PD17307. In another most preferred embodiment, the differentiation medium comprises a fifth differentiation medium comprising about 100 ng/mL Activin A, about 10 μM SU5402, and about 1.5 m CHIR99021, preferably comprising about 100 ng/mL Activin A, about 1 μM PD17307, and about 1.5 m CHIR99021. In another most preferred embodiment, the differentiation medium comprises another fifth differentiation medium being applied after the first fifth differentiation medium has been applied in the differentiation method comprising about 100 ng/mL Activin A, about 10 μM SU5402, and about 3 μM CHIR99021, preferably comprising about 100 ng/mL Activin A, about 1 μM PD17307, and about 3 μM CHIR99021.

In a preferred embodiment of the invention, when culturing the iPS cell in the differentiation method, it comprises culturing for about 2 days in the first differentiation medium as defined elsewhere herein, preferably for about 2 consecutive days in the first differentiation medium as defined elsewhere herein. This means that the iPS cell was first exposed to the first differentiation medium as defined herein from day 0 to day 2.

In another preferred embodiment, when culturing the iPS cell in the method of the invention, it comprises culturing for about 2 days in the second differentiation medium as defined elsewhere herein, preferably it comprises culturing for about 2 days in the first differentiation medium as defined elsewhere herein, subsequently culturing for about 2 days in the second differentiation medium as defined elsewhere herein, even more preferably it comprises culturing for about 2 consecutive days in the first differentiation medium as defined elsewhere herein, subsequently culturing for about 2 consecutive days in the second differentiation medium as defined elsewhere herein. This means on day 2 of the culturing of the iPS cell in the method of the invention, the iPS cell may be exposed to the second differentiation medium from day 2 to day 4.

In another preferred embodiment, when culturing the iPS cell in the method of the invention, it comprises culturing the iPS cell for about 2 days in the third differentiation medium as defined elsewhere herein, preferably it comprises culturing the iPS cell for about 2 days in the first differentiation medium as defined elsewhere herein, subsequently culturing for about 2 days in the second differentiation medium as defined elsewhere herein, subsequently culturing for about 2 days in the third differentiation medium as defined elsewhere herein, even more preferably it comprises culturing the iPS cell for about 2 consecutive days in the first differentiation medium as defined elsewhere herein, subsequently culturing for about 2 consecutive days in the second differentiation medium as defined elsewhere herein, subsequently culturing for about 2 consecutive days in the third differentiation medium as defined elsewhere herein. This means on day 4 of the culturing of the iPS cell in the method of the invention, the iPS cell may be exposed to the third differentiation medium from day 4 to day 6.

In another preferred embodiment, when culturing the iPS cell in the method of the invention, it comprises culturing the iPS cell for about 2 days in the fourth differentiation medium as defined elsewhere herein, preferably it comprises culturing the iPS cell for about 2 days in the first differentiation medium as defined elsewhere herein, subsequently culturing for about 2 days in the second differentiation medium as defined elsewhere herein, subsequently culturing for about 2 days in the third differentiation medium as defined elsewhere herein, subsequently culturing for about 2 days in the fourth differentiation medium as defined elsewhere herein, even more preferably it comprises culturing the iPS cell for about 2 consecutive days in the first differentiation medium as defined elsewhere herein, subsequently culturing for about 2 consecutive days in the second differentiation medium as defined elsewhere herein, subsequently culturing for about 2 consecutive days in the third differentiation medium as defined elsewhere herein, subsequently culturing for about 2 consecutive days in the fourth differentiation medium as defined elsewhere herein. This means on day 6 of the culturing of the iPS cell in the method of the invention, the iPS cell may be exposed to the fourth differentiation medium from day 6 to day 8.

In another preferred embodiment, when culturing the iPS cell in the method of the invention, it comprises culturing for about 8 days in the fifth differentiation medium as defined elsewhere herein, preferably it comprises culturing for about 2 days in the first differentiation medium as defined elsewhere herein, subsequently culturing for about 2 days in the second differentiation medium as defined elsewhere herein, subsequently culturing for about 2 days in the third differentiation medium as defined elsewhere herein, subsequently culturing for about 2 days in the fourth differentiation medium as defined elsewhere herein, and subsequently culturing for about 8 days in the fifth differentiation medium as defined elsewhere herein, even more preferably it comprises culturing for about 2 consecutive days in the first differentiation medium as defined elsewhere herein, subsequently culturing for about 2 consecutive days in the second differentiation medium as defined elsewhere herein, subsequently culturing for about 2 consecutive days in the third differentiation medium as defined elsewhere herein, subsequently culturing for about 2 consecutive days in the fourth differentiation medium as defined elsewhere herein, and subsequently culturing for about 8 consecutive days in the fifth differentiation medium as defined elsewhere herein. This means on day 8 of the culturing of the iPS cell in the method of the invention, the iPS cell may be exposed to the fifth differentiation medium from day 8 to day 16.

In an even more preferred embodiment of the invention, when culturing the iPS cell in the method of the invention, it comprises culturing for about 4 days in the fifth differentiation medium as defined elsewhere herein comprising CHIR99021 used in a concentration of at least about 1 μM and less than about 3 μM as defined elsewhere herein, followed by subsequently culturing for another about 4 days in the fifth differentiation medium comprising CHIR99021 used in a concentration of about 3 μM, most preferably it comprises culturing for about 2 days in the first differentiation medium, subsequently culturing for about 2 days in the second differentiation medium, subsequently culturing for about 2 days in the third differentiation medium, subsequently culturing for about 2 days in the fourth differentiation medium, and subsequently culturing for about 4 days in the fifth differentiation medium comprising CHIR99021 used in a concentration of at least about 1 μM and less than about 3 μM as defined elsewhere herein, followed by subsequently culturing for another about 4 days in the fifth differentiation medium comprising CHIR99021 used in a concentration of about 3 μM. In this context, the term “days” for culturing in a particular medium may also be exchanged with the term “consecutive days”. This means that on day 8 of the culturing in the method of the invention, the cell may be exposed to the fifth differentiation medium comprising CHIR99021 used in a concentration of at least about 1 μM and less than about 3 μM as defined elsewhere herein from day 8 to day 11, followed by exposing the cells on day 11 to the fifth differentiation medium comprising CHIR99021 used in a concentration of about 3 μM from day 11 to day 16.

The present invention thus also comprises the method of differentiating an iPS cell into a RPE cell, wherein the iPS cell is cultured in the differentiation medium in total for about 11 to about 21 days, for about 12 to about 20 days, for about 13 to about 19 days, for about 14 to about 18 days, for about 15 to about 17 days, preferably for about 16 days in total, even more preferably for about 11 to about 21 consecutive days, for about 12 to about 20 consecutive days, for about 13 to about 19 consecutive days, for about 14 to about 18 consecutive days, for about 15 to about 17 consecutive days, most preferably for about 16 consecutive days.

In a further embodiment of the invention, the method of differentiating an iPS cell into a RPE cell further preferably comprises culturing the iPS cell in a mTESR1 medium before culturing the iPS cell in the differentiation medium as defined herein. CLiPs and as a reference cell line human ES cells may be grown on Matrigel coated tissue culture plate in mTeSR1 medium. When the cells attain about 90 to about 95% confluence, they are then exposed to the differentiation medium as defined elsewhere herein, preferably to the first, even more preferably to the first followed by the second, third, fourth and fifth differentiation medium as defined herein. In a preferred embodiment, the method of differentiating an iPS cell into a RPE cell further comprises culturing the iPS cell in a mTESR1 medium for about 1 to about 4 culture days before culturing the iPS cell in the differentiation medium as defined herein, more preferably before culturing the iPS cell in the first differentiation medium as defined herein, most preferably before culturing the iPS cell in the first differentiation medium as defined herein, followed by the second, third, fourth and fifth differentiation medium as defined herein.

In a further embodiment of the invention, the method of differentiating an iPS cell into a RPE cell preferably further comprises culturing the RPE cell in a retinal pigment epithelial maintenance (short: RPEM) medium. According to the present invention after having cultured iPS cells in the differentiation medium, which then differentiated into RPE cells, the differentiation medium may be replaced by RPEM medium as will be defined below. Preferably, culturing RPE cell in said RPEM medium may start after day 16 of the culture (in particular after culturing the cells in the fifth differentiation medium as defined elsewhere herein). In a preferred embodiment, said RPEM medium of the differentiation method of the invention comprises about 50% DMEM/F12 and about 50% minimum essential medium (MEM) comprising 0.5× N1 supplement and 1× NEAA. In a more preferred embodiment, said RPEM medium of the differentiation method of the invention further comprises at least any one of a heat-inactivated fetal bovine serum (FBS), Glutamax, taurine, hydrocortisone, 3,3′,5-Triiodo-L-thyronine, penicillin/streptomycin, nicotinamide, or sodium pyruvate. In an even more preferred embodiment, said RPEM medium of the differentiation method of the invention further comprises a heat-inactivated fetal bovine serum (FBS), Glutamax, taurine, hydrocortisone, 3,3′,5-Triiodo-L-thyronine, penicillin/streptomycin, nicotinamide, and sodium pyruvate. In a most preferred embodiment, said RPEM medium of the differentiation method of the invention further comprises about 2% heat-inactivated fetal bovine serum (FBS), 1× Glutamax, about 0.25 mg/mL taurine, about 0.02 g/mL hydrocortisone, about 0.013 ng/mL 3,3′,5-Triiodo-L-thyronine, 1× penicillin/streptomycin, about 10 mM nicotinamide and 1× sodium pyruvate.

The present invention also comprises said differentiation method as defined elsewhere herein, wherein the RPE cell is cultured in said RPEM medium as defined elsewhere herein for about 9 to about 29 days, for about 10 to about 28 days, for about 11 to about 27 days, for about 12 to about 26 days, for about 13 to about 25 days, for about 14 to about 24 days, for about 15 to about 23 days, for about 16 to about 22 days, for about 17 to about 21 days, for about 18 to about 20 days, preferably for about 19 days, even more preferably for about 19 consecutive days. The RPEM medium may be changed every about 2 to about 3 days during the culturing of said RPE cells in said medium, preferably every about 2 to about 3 days during the culturing of said RPE cells for about 9 to about 29 days, for about 10 to about 28 days, for about 11 to about 27 days, for about 12 to about 26 days, for about 13 to about 25 days, for about 14 to about 24 days, for about 15 to about 23 days, for about 16 to about 22 days, for about 17 to about 21 days, for about 18 to about 20 days, more preferably during the culturing of said RPE cells for about 19 days, even more preferably for about 19 consecutive days.

The present invention also comprises said differentiation method as defined elsewhere herein, wherein culturing the iPS cell in the differentiation medium as defined herein and culturing the RPE cell in the RPEM medium as defined herein comprises about 20 to about 50 days, about 25 to about 45 days, about 30 to about 40 days, preferably about 30 to about 35 days, most preferably about 35 days, in particular having about 16 days of culturing iPS cells in the differentiation medium as defined elsewhere herein and having about 19 days of culturing the differentiated RPE cells in the RPEM medium as defined elsewhere herein.

The present invention also comprises said differentiation method as defined elsewhere herein further preferably comprising purifying the RPE cell in the RPEM medium after culturing said RPE cell in said RPEM medium. After differentiation there is a mixture of RPE cells and non-RPE cells, why a further purifying step can be helpful, so that differentiation plate may have only pure RPE cells (FIG. 20). The additional purification step of the differentiation method preferably comprises a) manually identifying the RPE cell according to their pigmentation, wherein manually identifying the RPE cell according to their pigmentation preferably comprises selecting by microscopy, more preferably selecting by bright field microscopy as known to a person skilled in the art. This step may refer to a manual purification of RPE cells. In particular, manually identifying the RPE cell according to their pigmentation may be performed by removing non-RPE cells, which have lesser pigmentation and different cell morphology than RPE cells, manually by scraping with a tip attached to the pipette, while observing through microscopy, such as bright field microscopy. It may further comprise washing, in particular with PBS for about 3 times, to remove all non-RPE cells. Additionally or alternatively, the additional purification step of the differentiation method preferably comprises b) passaging the RPE cell, wherein passaging the RPE cells preferably comprises treating the RPE cell with Accutase or TrypLE, most preferably with TrypLE. This step may refer to a passaging purification of RPE cells. In particular, passaging the RPE cell may be performed by detaching non-RPE cells and removing them by treatment with a gentle dissociation agent such as Accutase or TrypLE, preferably with TrypLE. The RPE cells may still be attached to the culture plate. It may then further comprise treating the remaining RPE cells again with a gentle dissociation agent such as Accutase or TrypLE, preferably with TrypLE and further passaging said RPE cells. In this context, the term “passaging RPE cell” refers to plating of the remaining RPE cell after having detached and removed non-RPE cell with a gentle dissociation agent such as Accutase or TrypLE, preferably with TrypLE and treating said remaining RPE cell again with a gentle dissociation agent such as Accutase or TrypLE, preferably with TrypLE. If TrypLE is applied, said step may refer to a TrypLE purification of RPE cells. Additionally or alternatively, the additional purification step of the differentiation method preferably comprises c) the combination of manually identifying the RPE cell according to their pigmentation as defined elsewhere herein and passaging the RPE cell as defined elsewhere herein. Although the purification performed by passaging RPE cell, preferably using the TrypLE purification described above, may remove most of the non-RPE cells/cell clumps, some small clumps may still be present which may be removed by manual purification as defined herein. Additionally or alternatively, the additional purification step of the differentiation method preferably comprises d) the combination of passaging the RPE cell and scatter sorting the RPE cell according to their pigmentation. This purification step may be performed by removing non-RPE cells as defined above and further treating said remaining RPE cell with a gentle dissociation agent such as Accutase or TrypLE, preferably with TrypLE and further processing the RPE cells in scatter sorting, wherein non-RPE cells removed by a gentle dissociation agent such as Accutase or TrypLE, preferably with TrypLE may be used to set the gate for scatter low cells. Additionally or alternatively, the additional purification step of the differentiation method preferably comprises e) the combination of scatter sorting the RPE cell according to their pigmentation. This step may refer to a scatter sorting purification of RPE cells. In particular, scatter sorting RPE cell may be performed by resuspending cell pellet of dissociated single cells (being dissociated by using a gentle dissociation agent such as Accutase or TrypLE, preferably TrypLE) in any FACS buffer and passing it through a filter to get single cells and separating into scatter high and low fractions using e.g. any FACS cell sorter known to a person skilled.

By comparing different RPE purification methods as defined herein, it was found that passaging the RPE cell comprising treating the RPE cell with Accutase or TrypLE, preferably with TrypLE as defined above, gives high RPE yield (about 47%) and is easier and faster compared to the manual purification of RPE cells and may thus be preferred. Passaging the RPE cell comprising treating the RPE cell with Accutase or TrypLE, preferably with TrypLE as defined above, followed by manually identifying the RPE cell according to their pigmentation as also defined elsewhere herein, not only gives high RPE yield (about 43%), but also the highest purity of the cells (about 99% PMEL17 positive cells), which may be of most importance for transplantation. Additionally, passaging the RPE cell comprising treating the RPE cell with Accutase or TrypLE, preferably with TrypLE as defined above, followed by manually identifying the RPE cell according to their pigmentation as also defined elsewhere herein, is also the easiest to perform as partial TrypLE treatment removes majority of the non-RPE cells in a short amount of time needed for purification. In sum, the purification of passaging the RPE cell comprising treating the RPE cell with Accutase or TrypLE, preferably with TrypLE, in combination with manually identifying RPE cells, involves an additional manual purification step to remove any non-RPE cells that might have escaped the treatment with a gentle dissociation agent such as Accutase or TrypLE, preferably with TrypLE and is mostly preferred as purification of RPE cells in the method of the present invention.

The present invention also comprises said differentiation method as defined elsewhere herein, wherein the iPS cell, which is derived from a stem cell of the amniotic membrane of the umbilical cord and used in the method of differentiating into a RPE cell, is particularly generated by expressing exogenous nucleic acids encoding the proteins OCT3/4, SOX2, KLF4, LIN28 and L-MYC and the p53-shRNA in said stem cell of the amniotic membrane of the umbilical cord under conditions suitable to reprogram the stem cell as defined elsewhere herein for the method of generating an induced pluripotent stem cell also comprised by the present invention. In a preferred embodiment, the present invention also comprises said differentiation method as defined elsewhere herein, wherein the exogenous nucleic acids encoding the proteins OCT3/4, SOX2, KLF4, LIN28 and L-MYC and the p53-shRNA in said stem cell of the amniotic membrane of the umbilical cord are provided by one, two or three vectors, wherein preferably a first vector encodes the protein OCT3/4 and the 53-shRNA, a second vector encodes the proteins SOX2 and KLF4 and a third vector encodes the proteins L-MYC and LIN28. In sum, each disclosure regarding the method of generating an induced pluripotent stem cell as defined elsewhere herein, which iPS cell is then differentiated into a RPE cell according to the described differentiation method, may be applicable, where necessary also to the method of differentiating an iPS cell into a RPE cell.

The present invention is also directed to a RPE cell culture/a RPE cell obtainable by the differentiation method as described herein and to a RPE cell culture/a RPE cell obtained by the differentiation method as described herein. The present invention is also directed to a retinal pigment epithelium consisting of or comprising a retinal pigment epithelial cell culture/a RPE cell obtainable or obtained by the differentiation method as described herein.

The present invention is also directed to a pharmaceutical composition comprising a RPE cell culture/a RPE cell obtainable/obtained by the differentiation method as described herein. An example for a pharmaceutical composition comprising differentiated RPE cell/RPE cell culture comprising differentiated RPE cell is an injection solution or any kind of graft suitable for implanting the differentiated RPE cell. When the pharmaceutical composition is an injection solution, said composition may comprise the RPE cell culture obtainable/obtained by the differentiation method as described herein. When the pharmaceutical composition is a graft suitable for implanting, said composition may comprise an implantable matrix, preferably a polyester matrix, even more preferably a polyester matrix in a transwell, said matrix coated with said differentiated RPE cells obtainable/obtained by the differentiation method as described herein which may have been grown on said matrix. In this context, the RPE cells, grown on said matrix as defined herein, may refer to said RPE cell culture obtainable/obtained by the differentiation method as described herein. The pharmaceutical composition may be formulated/adapted for parenteral or local application as known to a person skilled. In such case, the parenteral application may comprise a sterile preparation intended for injection, infusion or implantation in the human or animal body. Local application as used herein preferably refers to subretinal application. If the pharmaceutical composition refers to a graft as defined above with regard to the implantable matrix, said composition may be formulated/adapted for subretinal (under the retina of the eye) application, in other words may be transplanted subretinally. The present invention also comprises a diagnostic composition for research purposes comprising the RPE cells (culture) and Matrigel. Said composition also refers to a graft suitable for implanting into a subject as defined herein, wherein said RPE cells differentiated by the method as defined herein are mixed up with Matrigel and then the graft (cells in the Matrigel) is implanted into the subject. If the diagnostic composition refers to a graft as defined above with regard to the Matrigel, said composition may be formulated/adapted for subcutaneous application, in other words may be transplanted subcutaneously.

A RPE cell culture obtainable/obtained by the differentiation method as described herein, also comprised in a retinal pigment epithelium, and/or also comprised by a pharmaceutical composition as defined elsewhere herein, may refer to a plurality of a RPE cells, which are obtainable/obtained by the differentiation method and preferably comprising a culture medium for said RPE cells. The term “population” may also be used interchangeably with the term “culture”. Said differentiated RPE cell obtainable/obtained by the differentiation method of the present invention, which is comprised in said RPE cell culture can be further characterized: Said differentiated RPE cell firstly may comprise higher % areas of pigmentation compared to skin iPS-derived RPE. By visual grading, all fours CLiPS tested by the invention, which may be selected from the group consisting of CLMC23, CLMC30, CLMC44 and CLEC23 developed about 30 to about 100%, about 50 to about 100%, about 70 to about 100% pigmented RPE cells using the differentiation medium as defined herein, compared to only 30% of the skin iPS cells (such as Asf5, AGO and/or HDFA) achieving similar pigmentation (FIG. 13). Further, said differentiated RPE cell, additionally comprised in said culture, may express at least any one of BEST1, PMEL17, MITF, TYROSINASE, TRYP2, ZO-1, RPE65, RLBP1 or MERTK or a combination of all of the protein markers listed (FIGS. 15, 16, and 18). In one embodiment, said differentiated RPE cell, additionally comprised in said culture, may express the protein marker BEST1. In another embodiment, said differentiated RPE cell, additionally comprised in said culture, may express the protein marker PMEL17. In another embodiment, said differentiated RPE cell, additionally comprised in said culture, may express the protein marker MITF. In another embodiment, said differentiated RPE cell, additionally comprised in said culture, may express the protein marker TYROSINASE. In another embodiment, said differentiated RPE cell, additionally comprised in said culture, may express the protein marker TRYP2. In another embodiment, said differentiated RPE cell, additionally comprised in said culture, may express the protein marker ZO-1. In another embodiment, said differentiated RPE cell, additionally comprised in said culture, may express the protein marker RPE65. In another embodiment, said differentiated RPE cell, additionally comprised in said culture, may express the protein marker RLBP1. In another embodiment, said differentiated RPE cell, additionally comprised in said culture, may express the protein marker MERTK. CLiPS-derived RPE cell is thus more highly pigmented compared to ES-derived RPE cells such as H9 cells used herein. This is associated with higher expression of pigmentation associated genes such as for example MITF, PMEL17, TYROSINASE and TRYP2 (FIG. 15). Further, said differentiated RPE cell, additionally comprised in said culture, can be characterized by lacking or having a reduced expression of the cell cycle proliferation marker Ki67. The expression of the mature RPE marker RPE65 and Ki67 as proliferation marker being absent, confirms the mature and quiescent state which reflects the survival rate of such RPE cells differentiated from CLiPS according to the method as defined elsewhere herein (FIG. 22).

In more detail, the RPE cell obtainable/obtained by the method as defined herein can further be characterized by expressing BEST1 with a fold change of at least about 2, at least about 2.1, at least about 2.2, at least about 2.3, at least about 2.4, at least about 2.5, at least about 2.6, at least about 2.7, at least about 2.8, at least about 2.9, at least about 3 or more, preferably of about 3 relative to a RPE cell differentiated from an embryonic stem cell (ES) (cell or culture from which it has been generated). Additionally or alternatively, the RPE cell obtainable/obtained by the method as defined herein can further be characterized by expressing PMEL17 with a fold change of at least about 0.9, at least about 0.91, at least about 0.92, at least about 0.93, at least about 0.94, at least about 0.95, at least about 1, at least about 1.1, at least about 1.2, at least about 1.3 or more relative, preferably of about 1.3 to a RPE cell differentiated from an ES (cell or culture from which it has been generated). CLiPS-RPE thus comprise a higher % of PMEL17 positive cells after differentiation. It was found that the CLiPS used (such as CLMC23, CLMC30 and CLEC23) in the differentiation method comprises about 89% to about 95% purity of RPE cells. In contrast, only one of the 3 skin iPS cells used such as Asf5, AGO and/or HDFA had above about 90% purity (FIG. 14). Additionally or alternatively, the RPE cell obtainable/obtained by the method as defined herein can further be characterized by expressing MITF with a fold change of at least about 4.5, at least about 5, at least about 5.5, at least about 6, at least about 6.5, at least about 6.6, at least about 6.7, at least about 6.8 or more, preferably of about 6.8 relative to a RPE cell differentiated from an ES (cell or culture from which it has been generated). Additionally or alternatively, the RPE cell obtainable/obtained by the method as defined herein can further be characterized by expressing TRYP2 with a fold change of at least about 2.9, at least about 3, at least about 3.5, at least about 4, at least about 4.1, at least about 4.2, at least about 4.3 or more, preferably of about 4.3 relative to a RPE cell differentiated from an ES (cell or culture from which it has been generated). Additionally or alternatively, the RPE cell obtainable/obtained by the method as defined herein can further be characterized by expressing RPE65 with a fold change of at least about 0.6, at least about 0.7, at least about 0.8, at least about 0.9, at least about 0.91, at least about 0.92, at least about 0.93, at least about 0.94, at least about 0.95, at least about 0.96 or more, preferably of about 0.96 relative to a RPE cell differentiated from an ES (cell or culture from which it has been generated). Additionally or alternatively, the RPE cell obtainable/obtained by the method as defined herein can further be characterized by expressing RLBP1 with a fold change of at least about 17.5, at least about 18, at least about 19, at least about 20, at least about 21, at least about 22, at least about 23, at least about 24, at least about 25, at least about 26, at least about 26.1, at least about 26.2 or more, preferably of about 26.2 relative to a RPE cell differentiated from an ES (cell or culture from which it has been generated). Additionally or alternatively, the RPE cell obtainable/obtained by the method as defined herein can further be characterized by expressing MERTK with a fold change of at least about 6, at least about 6.5, at least about 7, at least about 7.5, at least about 8, at least about 8.5, at least about 9, at least about 9.1 or more, preferably of about 9.1 relative to a RPE cell differentiated from an ES (cell or culture from which it has been generated) (FIG. 20k). In this context, such ES cell which is used for comparison and where a RPE cell has also been differentiated from, may refer to a H9 ES cell.

In this context, the term “relative to” or “to” can also be replace with the term “compared to”, bringing two cells (for example CLMC23 as tested cell and H9 as reference cell) in comparison to one another, e.g. with regard to gene expression expressed as fold change of the tested cell to the reference cell. A “fold change” as used herein is a measure describing how much a quantity changes going from an initial to a final value. For example, an initial value of 30 and a final value of 60 correspond to a fold change of 2, or in common terms, a two-fold increase. Fold change is calculated simply as the ratio of the final value to the initial value, i.e. if the initial value is A and final value is B, the fold change is B/A. The fold-change can be obtained with respect to mRNA levels of the markers as described herein. Such fold-changes may be measured using RT-qPCR.

Also it was found that CLiPS-derived RPEs may achieve trans-epithelial electrical resistance (short: TEER) similar to a RPE cell differentiated from an ES (cell or culture from which it has been generated) and/or to a RPE cell differentiated from a skin iPS (cell or culture from which it has been generated). CLiPS-RPE also showed high phagocytosis similar to a RPE cell differentiated from an ES (cell or culture from which it has been generated) and/or to a RPE cell differentiated from a skin iPS (cell or culture from which it has been generated) (FIG. 17).

Additionally or alternatively, the RPE cell obtainable/obtained by the method as defined herein can further be characterized by comprising an increased oxygen consumption rate (OCR) and/or extracellular acidification rate (ECAR) relative to a RPE cell differentiated from an ES and/or relative to a RPE cell differentiated from a skin iPS. These features refer to the bioenergetics of said RPE demonstrating increased glycolysis and/or mitochondrial respiration. Glycolytic function was quantified by ECAR and oxidative phosphorylation (oxPhos) by OCR (FIG. 21).

In this context, OCR may comprise basal respiration, ATP production, maximal capacity and/or spare respiratory capacity. The term “increased” in this context with regard to OCR means that the OCR of RPE cell is increased by at least about 30%, at least about 31%, at least about 32%, at least about 33%, at least about 34%, at least about 35%, preferably at least about 35%; or by between about 30 to about 45%, by between about 31 to about 44%, by between about 32 to about 43 by between about 33 to about 42%, by between about 35 to about 40% relative to a RPE cell differentiated from an ES and/or relative to a RPE cell differentiated from a skin iPS. In more detail, i) the basal respiration of RPE cell is increased by about 38% relative to a RPE cell differentiated from an ES and/or relative to a RPE cell differentiated from a skin iPS; ii) the ATP production of RPE cell is increased by about 40% relative to a RPE cell differentiated from an ES and/or relative to a RPE cell differentiated from a skin iPS; iii) the maximal capacity of RPE cell is increased by about 35% relative to a RPE cell differentiated from an ES and/or relative to a RPE cell differentiated from a skin iPS; iv) the spare respiratory capacity of RPE cell is increased by about 36% relative to a RPE cell differentiated from an ES and/or relative to a RPE cell differentiated from a skin iPS.

Further, in this context, ECAR may comprise glycolysis, glycolytic capacity and/or glycolytic reserve. The term “increased” in this context with regard to ECAR means that the ECAR of RPE cell is increased by at least about 20%, at least about 21%, at least about 22%, at least about 23%, at least about 24%, at least about 25%, at least about 30%, at least about 35%, at least about 40%, at least about 45%, at least about 50%; or by between about 20 to about 55%, by between about 25 to about 55%, by between about 25 to about 50 relative to a RPE cell differentiated from an ES and/or relative to a RPE cell differentiated from a skin iPS. In more detail, i) the glycolysis of RPE cell is increased by about 25% relative to a RPE cell differentiated from an ES and/or relative to a RPE cell differentiated from a skin iPS; ii) the glycolytic capacity of RPE cell is increased by about 37% relative to a RPE cell differentiated from an ES and/or relative to a RPE cell differentiated from a skin iPS; iii) the glycolytic reserve of RPE cell is increased by about 50% relative to a RPE cell differentiated from an ES and/or relative to a RPE cell differentiated from a skin iPS.

Further, additionally or alternatively, the RPE cell obtainable/obtained by the method as defined herein can further be characterized by having/comprising less immunogenicity as defined in more detail below relative to a RPE cell differentiated from an ES (cell or culture from which it has been generated) and/or relative to a RPE cell differentiated from a skin iPS (cell or culture from which it has been generated). In this context and also with regard to the in vivo method as defined below, less immunogenicity refers to the RPE cell, preferably pre-delivered to a subject from whom a sample comprising said RPE cell has been obtained from for further analysis of said cells, has a reduced systemic immune response which may refer to (a) reduced level(s) of (a) pro-inflammatory cytokine(s) involved in induction of cellular immune response, preferably of IFN-γ and/or IL-18 (as a surrogate for cellular immune response) and/or of IL-23 and/or IL17A, cytokines involved in T cell activation, said reduced cytokine level being generated by said subject to whom said RPE cell has been pre-delivered before, in particular by said immune cells present in said subject and which are comprised also within said sample. Such reduced systemic immune response in said RPE cell may also refer to a decreased accumulation of immune cells, preferably at the site of injection as defined in the Example section. A reduced systemic immune response may thus comprise the RPE cell as defined herein having reduced systemic T cell activation, preferably reduced/suppressed CD8 cytotoxic T cell activation relative to a RPE cell differentiated from an ES and/or relative to a RPE cell differentiated from a skin iPS.

The present invention further includes a method of treating a retinal degenerative disease in a subject, wherein the subject may be selected from the group comprising a mouse, a rat, a rabbit, a pig, a dog, a cat, a non-human primate (monkey) or a human. In a preferred example, the subject is human. In this context, treating may comprise administering to a subject as defined herein a RPE cell differentiated from CLiPS by the differentiation method as described herein and/or said RPE cell culture obtained by the method of the invention and/or said pharmaceutical composition as defined herein. That said differentiated cell/culture obtainable/obtained by the method as defined herein is suitable for administration in said particular treatment of a retinal degenerative disease, is demonstrated by the fact that said RPE cell comprises hypoimmunogenic properties such as that reduced levels of pro-inflammatory cytokines, preferably of IFN-γ and/or IL-18 as a surrogate are generated, and having reduced cellular immune response, preferably after pre-injection of said differentiated RPE cells into a subject as defined herein (FIG. 23). This demonstrates that said RPE cells of the present invention may reduce immune cell infiltration at the localized site of RPE cell injection into a subject. Also, it was demonstrated that the sample obtained from a subject to whom said differentiated RPE cell has been pre-delivered before the cytokine analysis, comprises decreased levels of IL-23 and/or IL17A, cytokines involved in T cell activation, relative to a sample obtained from a reference subject to whom RPE cell differentiated from an ES has been pre-delivered before and/or relative to a sample obtained from a reference subject to whom a RPE cell differentiated from a skin iPS has been pre-delivered before. In sum, said RPE cell as defined herein may be capable that reduced levels of IL-23 and/or IL17A as another preferable example of pro-inflammatory cytokines are generated as defined herein. Also, T cell activation, in particular CD8 cytotoxic T cell activation, may be suppressed in a sample from a subject comprising said RPE cell of the invention. In other words, said RPE cell may suppress T cell activation, in particular CD8 cytotoxic T cell activation (FIG. 24). The degenerative disease being treated is a disease of the retina known to a person skilled, preferably the retinal degenerative disease is age-related macular degeneration (AMD) or retinal dystrophy. In one embodiment, the present invention refers to a method of treating AMD in a subject as defined herein, comprising administering to a subject a RPE cell differentiated from an iPS cell obtained by the method as defined herein. In another embodiment, the present invention refers to a method of treating retinal dystrophy in a subject as defined herein, comprising administering to a subject a RPE cell differentiated from an iPS cell obtained by the method as defined herein. Administration of said RPE cell differentiated from CLiPS in the method of treatment may include parenteral or local (preferably subretinal) application as known to a person skilled.

The present invention further includes an in vivo method of detecting the survival rate of a RPE cell differentiated from an iPS cell by the differentiation method as defined elsewhere herein in a subject, the method comprising a step a) introducing a RPE cell differentiated from an iPS cell by the method as defined herein into a subject, wherein said RPE cell comprises a bioluminescence label.

In this context, the term “survival rate” refers to the RPE cell not having died and still being mature and/or comprising a quiescent state, which may be confirmed by detecting the expression of RPE65 as mature RPE marker and by detecting no expression of Ki67 as proliferation marker, after said cells have been introduced into said subject as defined herein and detected over an amount of time as further described. Monitoring the survival rate can also be used interchangeably herein with regard to the in vivo method as defined.

The term “introduce” or “introducing” in step a) refers to bringing said RPE cell as defined herein into said subject, preferably by transplanting said RPE cell into said subject, even more preferably by transplanting said RPE cell subcutaneously into said subject with regard to using a mouse as a subject and a Matrigel plug assay as defined elsewhere herein. The term “subject” when used herein according to the in vivo method and also to the in vitro screening method includes mammalian and non-mammalian subjects. Preferably, the subject is an animal. The subject of the in vivo and in vitro method may refer to a mammal, including human, domestic and farm animals, non-human primates, and any other animal that has mammary tissue. In some embodiment the mammal is a mouse. In some embodiment the mammal is a rat. In some embodiment the mammal is a guinea pig. In some embodiment the mammal is a rabbit. In some embodiment the mammal is a cat. In some embodiment the mammal is a dog. In some embodiment the mammal is a monkey. In some embodiment the mammal is a horse. In a preferred embodiment the mammal/the animal as the subject used in said methods of the present invention is a mouse. In a most preferred embodiment the mammal/the animal as the subject used in said methods is a humanized mouse.

The RPE cell being differentiated according to the present invention and being introduced into said subject as defined herein within the in vivo method, comprises a bioluminescence label. In this context, the term “label” may be a fluorescent label or an enzyme suitable for bioluminescence. When the label is fluorescent label it can be a fluorophore (also called fluorochrome or chromophore). Such fluorophore may be any one of a fluorescent dye such as but not limited to Fluorescein (FITC), Alexa Fluor 350, 405, 488, 532, 546, 555, 568, 594, 647, 680, 700, 750, Pacific Blue, Coumarin, Pacific Green, Cy3, Texas Red, PE, PerCP-Cy5, PE-Cy7, Pacific Orange, or a fluorescent protein such as R-PE or APC, or an expressed fluorescent protein such as CFP, EGFP, GFP or RFP. When the label is an enzyme it can be, but is not limited to, a luciferase, preferably selected from the group consisting of bacterial luciferase (\uxAB), photinus luciferase, ren/7/a luciferase, and firefly luciferase. In a preferred embodiment, said RPE cell being differentiated according to the present invention and being introduced into said subject as defined herein within the in vivo method, comprises a bioluminescence enzyme gene-encoding vector, preferably tagged with an expressed fluorescent protein as defined herein. In a most preferred embodiment, said RPE cell being differentiated according to the present invention and being introduced into said subject as defined herein within the in vivo method, comprises a luciferase enzyme gene-encoding vector, preferably tagged with GFP. Alternatively or additionally, also comprised by step a) of said in vivo method as defined herein is the introduction of said RPE cell differentiated by the method as defined herein into a subject, the RPE cell being comprised in a RPE-matrigel plug as known to a person skilled in the art. Being comprised in a Matrigel plug means a mixture/composition of said RPE cells with Matrigel as known to a person skilled in the art.

The in vivo method as defined herein further comprises step b) detecting the bioluminescence signal of said RPE cell over time using an imaging method, thereby collecting imaging data.

The terms “detecting or detection” (may also refer to “monitoring”) when used herein with regard to the in vivo method refers to the visualization and the qualitative analysis of the bioluminescence of said RPE cells in vivo using any known imaging method, preferably if the bioluminescence label is luciferase using the bioluminescent imaging method. Said detection of a bioluminescence signal of said RPE cell is according to step b) of the in vivo method performed over time which may refer to at least about 2 days, about 7 days, about 10 days, about 14 days, about 17 days, about 21 days, about 24 days, about 28 days, about 35 days, about 42 days, about 49 days, or at least about 56 days; or between about 2 to about 56 days, or between about 2 to about 49 days. Said detection of the bioluminescence is preferably monitored at regular intervals over said time course as defined herein. The detection of bioluminescence may refer to the detection of total radiance of said cells expressed in p/s/cm2/sr as known to a person skilled in the art. By said detection of said bioluminescence signal over time imaging data are collected.

The in vivo method as defined herein further comprises step c) comparing the imaging data received in step b) to reference imaging data. In this context, the reference imaging data refer to the bioluminescence signal(s) (“imaging signal”) of RPE cells being differentiated from an ES cell (preferably H9 cell) and/or differentiated from a skin iPS (preferably HDFA or ASF5) which has/have also been detected over time as defined herein. In this context, said RPE cells differentiated from an ES cell and/or differentiated from a skin iPS have also been introduced as defined herein into a subject (e.g. different mouse), which is not the exact same subject as the subject to whom the RPE cells have been pre-delivered as defined herein (e.g. mouse), and wherein said RPE cells being differentiated from an ES cell and/or differentiated from a skin iPS also comprise the bioluminescence label as defined herein as for the CLiPS derived RPE cells.

The present invention also comprises the in vivo method as defined herein, wherein no difference in the bioluminescence signal in the imaging data as compared to the reference imaging data indicates survival of said RPE cell in said subject. No difference also comprises slight, but insignificant decrease in the bioluminescence signal over time in the imaging data compared to the reference imaging data of said RPE cells differentiated from an ES cell and/or differentiated from a skin iPS.

The present invention also comprises an animal comprising said RPE cell obtained/obtainable by the differentiation method as defined elsewhere herein. In this context, said “animal” refers to any mammal as defined herein, preferably a mouse, most preferably a humanized mouse. By the animal comprising said RPE cell, it may be meant that said RPE cell obtained/obtainable by the differentiation method as defined elsewhere herein is introduced to said animal as defined herein, preferably by transplanting said RPE cell subcutaneously.

The introduction of said RPE cells differentiated from iPS cell by the differentiation method in vivo to said subject as defined herein and the subsequent in vitro analysis of a sample comprising said RPE cell obtained from said subject may be particularly envisaged for the analysis of such cells in various models for research and development purposes.

Thus, the present invention also comprises an in vitro (screening) method of determining the immunogenicity of a RPE cell differentiated from an iPS cell by the method as defined herein in a subject, to whom said differentiated RPE cell has been pre-delivered, the method comprising a step a) detecting a pro-inflammatory cytokine level using an imaging method, in a sample which is obtained from said subject defined herein, wherein the sample comprises said differentiated RPE cell, thereby collecting imaging data. “Pre-delivered” includes in this regard, that the differentiated RPE cell of the present invention has been delivered to the subject as defined herein prior to the in vitro screening method. After that the sample comprising the differentiated RPE cell is obtained from said subject and the sample further analyzed, e.g. for particular cytokine levels. The term “pre-introduced” may also be used interchangeably.

In this context, detecting refers to the visualization and the quantative analysis of said cytokine levels as defined herein in vitro using any known imaging method suitable for detecting cytokines such as flow cytometry. Said cytokine level being detected as defined herein for the in vitro method refers, but is not limited to, a level of a cytokine associated with cell-mediated immunity, preferably to the level of the pro-inflammatory cytokine IFN-γ, IL-18, IL-23 and/or IL17A. The level of said cytokine as defined herein may be expressed in μg/ml.

The sample being obtained from said defined subject in step a) of the in vitro method may be any biological sample taken from said subject, preferably blood serum sample.

The in vitro method may comprise as an additional or as an alternative step within said method detecting immune cell infiltration in a sample obtained from said subject as defined herein, the sample comprising said differentiated RPE cell, thereby further collecting imaging data.

The method further comprises a step b) comparing the imaging data received in step a), and/or received from the detection step with regard to the infiltration, to reference imaging data. Again, said reference imaging data refer to imaging data of the same detected pro-inflammatory cytokine level as defined herein within a sample from a subject to whom RPE cell differentiated from an ES cell and/or differentiated from a skin iPS as reference cell has been pre-delivered and/or to imaging data of the same immune cell(s) being infiltrated and detected within a sample from a subject to whom RPE cell differentiated from an ES cell and/or differentiated from a skin iPS as reference cell has been pre-delivered. Said reference cell may be comprised also in the same kind of sample (e.g. blood sample, but different blood sample) as the sample which comprises the RPE cell, but the reference sample may be obtained from a different subject (e.g. a different mouse) compared to the test subject from which the sample comprising the RPE cell has been obtained from. Preferably, a decreased cytokine level and/or reduced/decreased immune cell infiltration/accumulation in the imaging data as compared to reference imaging data indicates reduced immunogenicity of said RPE cells in said subject meaning the ability of said RPE cell according to the invention to provoke an immune response in the subject is lower/reduced compared to the immunogenicity of a RPE cell differentiated from an ES cell and/or differentiated from a skin iPS.

The invention will be further illustrated by the following non-limiting Experimental Examples.

### EXPERIMENTAL EXAMPLES

### Example 1: Developing Suitable Electroporation Parameters for CLiPS

Electroporation according to the protocol described in Okita et al., supra, was found to be not working at all. No IPS colony was detected, when a reaction mixture of CLMC was electoporated with the episomal vectors pCXLE-hOCT3/4-shp53-F, pCXLE-hSK, and pCXLE-hUL, following the protocol of Okita et al, supra. For CLEC an average reprogramming efficiency (expressed in terms IPS colony counts) of only 0.2% after electroporation of CLMC with episomal vectors pCXLE-hOCT3/4-shp53-F, pCXLE-hSK, and pCXLE-hUL (Addgene plasmids #27077 (SEQ ID NO: 12—inter alia comprising SEQ ID NO: 11), #27078 (SEQ ID NO: 13), #27080 (SEQ ID NO: 14) was found, following the protocol of Okita et al, supra. Accordingly, it was necessary to either develop from scratch a suitable electroporation protocol for CLMC derived CLiPS method or, in the case of CLEC, to provide a significantly improved electroporation protocol. For this purpose, the electric parameters such as number of electric pulses, duration time and voltage, constituting electroporation, were varied to develop useable electroporation conditions for CLSC. In this experiment, numerous different electroporation settings were tested on individual CLMC and CLEC samples, respectively, cultivated under the cell specific conditions as described here. After each electroporation, about 200.000 cells were plated in triplicates in a 6-well plate for cultivation. About 21 days after electroporation, CLSC colonies that had developed by then were counted to determine the survival rate. The survival rate was used to draw conclusions about electroporation efficiency. The percentage efficiency has been calculated as Colony number/200,000×100%.

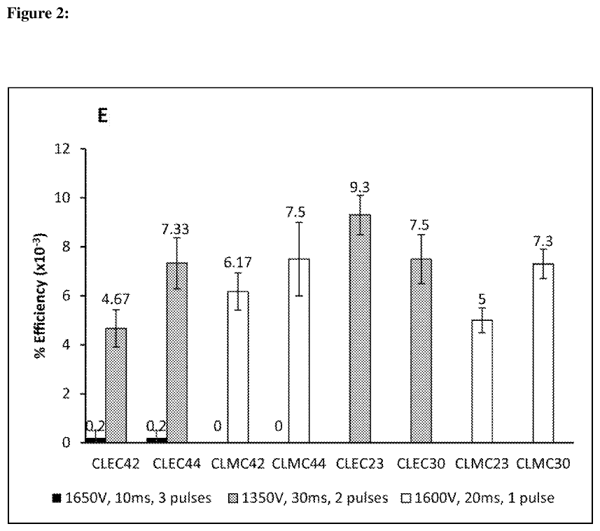

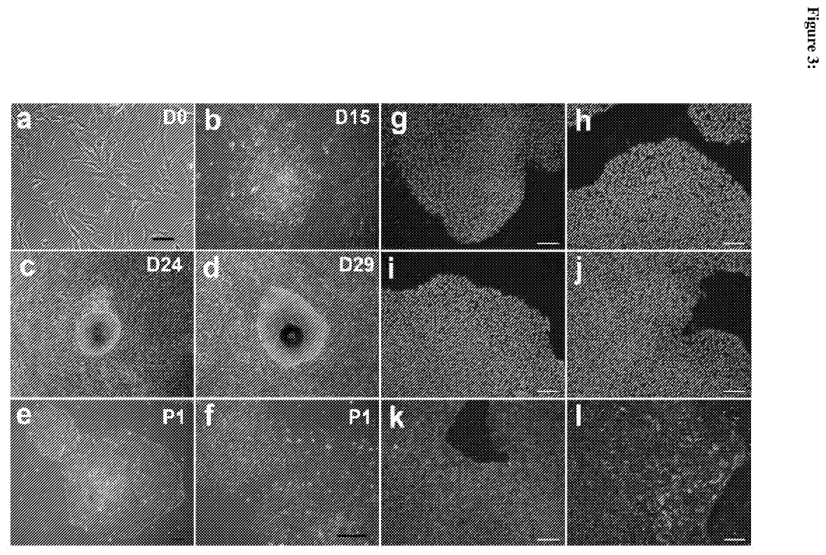

The results shown in Table 1 and FIG. 2 indicate that suitable electroporation conditions could be found for both CLMC and CLEC. The optimal electroporation setting for CLEC found here comprises 2 electric pulses each of 30 ms and 1350 V using an amount of 1.67 μg (plasmid) DNA of each of the three vectors (pCXLE-hOCT3/4-shp53-F, pCXLE-hSK, and pCXLE-hUL) for a number of 1×10⁶ cells. Four individual CLEC lines (CLEC42, CLEC44, CLEC23 and CLEC30) transfected with these settings exhibited a survival rate of 4.67%, 7.33%, 9.33% and 7.50%, respectively. In comparison to Okita et al., supra, the electroporation settings used for CLEC increased the electroporation efficiency about 23.35% for CLEC42 and about 36.65% for CLEC44. Thus, it has surprisingly been found that these electroporation parameters/settings increase the electroporation efficiency about 30% for CLEC on average compared to the conditions used by Okita et al for electroporation of human skin fibroblasts. Notably, the electroporation settings used here differ rather significantly from conditions reported for successful electroporation of epithelial cells such as corneal epithelial cells (1 electric pulse of 30 ms and 1300 V and a ratio of the amount of plasmid DNA (μg) to the number of cells (1×10⁶ cells) of 1:1 (cf. Png, E. et al. (2011), Journal of Cellular Physiology. United States, 226(3), pp. 693-699).

The effect of optimizing the electroporation protocol is even more significant regarding CLMC since, as stated above, electroporation according to Okita et al., supra, resulted in no surviving CLMC at all. It was found here that four individual CLMC lines (CLMC42, CLMC44, CLMC23 and CLMC30) were successfully transfected with 1 electric pulse of 20 ms and 1600 V and a ratio of the amount of plasmid DNA of each the three episomal vectors (pCXLE-hOCT3/4-shp53-F, pCXLE-hSK, and pCXLE-hUL) to the number of cells of 1.67 μg (plasmid) DNA to about 1×10⁶ CLMC. The resulting transgenic cells exhibited a survival rate of 6.17%, 7.50%, 5.00% and 7.33%, respectively. Notably, also the electroporation/transfection conditions found here to be the optimum for the generation of CLiPS from CLMC are different from electroporation conditions reported so far. Cf. in this context Sprangers, A. J., Freeman, B. and Ogle, B. M. (2011), pp. 62-66, for example, who examined possible negative effects of electroporation of human embryonic stem cell (hESC)-derived mesenchymal stem cells. So doing, Sprangers et al. found that transfecting a total of 4 μg (plasmid) DNA in 1×10⁶ mesenchymal stem cells using 1 electric pulse of 20 ms and 1400 V provided the optimum for MSC transfection. Thus, the present invention provides a unique and efficient protocol for CLEC and CLMC electroporation, respectively. The variations in the transfection efficiencies across the four individual CLSC lines (cells from different donors) are inter-individual variabilities being an inherent and documented feature of iPS derivation. To confirm the gender of the donor CLSC lines and CLiPS derived from them, a PCR amplification was performed with gene-specific primers on genomic DNA isolated from individual CLSC lines to determine the presence or absence of the DYS439 and SRY loci, which are both present on the Y chromosome. aSF4 adult skin fibroblasts, which is confirmed to be obtained from a male donor, was used as a positive control.

TABLE 1
Optimized electroporation conditions for generation of CLiPS

| | 1650 V, 10 ms, | Optimized CLEC | Optimized CLMC |
|---|---|---|---|
| | 3 pulses | protocol | protocol |
| | (Okita et al., 2011) | 1350 V, 30 ms, 2 pulses | 1600 V, 20 ms, 1 pulse |
| Ratio of | 1.67 μg/1 × 10⁶ cells | 1.67 μg/1 × 10⁶ cells | 1.67 μg/1 × 10⁶ cells |
| the | | | |
| amount of | | | |
| DNA to | | | |
| number of | | | |
| cells | | | |
| [μg/1 × 10⁶ | | | |
| cells] for | | | |
| each | | | |
| vector | | | |

| | Av. | Av. % | | Av. % | | Av. % |
|---|---|---|---|---|---|---|
| | Colony | Efficiency | Av. Colony | Efficiency | Av. Colony | Efficiency |
| | count | (×10⁻³) | count | (×10⁻³) | count | (×10⁻³) |
| CLEC42 | 0.33 ± 0.58 | 0.2 ± 0.3 | 9.33 ± 1.53 | 4.67 ± 0.76 | — | — |
| CLEC44 | 0.33 ± 0.58 | 0.2 ± 0.3 | 14.67 ± 2.08 | 7.33 ± 1.04 | — | — |
| CLMC42 | 0 | 0 | — | — | 12.33 ± 1.53 | 6.17 ± 0.76 |
| CLMC44 | 0 | 0 | — | — | 15 ± 3 | 7.50 ± 1.50 |
| CLEC23 | — | — | 18.67 ± 1.53 | 9.33 ± 0.77 | — | — |
| CLEC30 | — | — | 15.00 ± 2.00 | 7.50 ± 1.00 | — | — |
| CLMC23 | — | — | — | — | 10.00 ± 1.00 | 5.00 ± 0.50 |
| CLMC30 | — | — | — | — | 14.67 ± 1.15 | 7.33 ± 0.58 |

### Example 2: Derivation of Transgene Integration- and Feeder-Free Human iPS

Cord lining epithelial cells (CLEC) and cord lining mesenchymal cells (CLMC) were isolated and supplied by CellResearch Corporation Pte Ltd, Singapore. CLEC and CLMC were thawed and propagated in their culture medium PTT-e3 and PTT-4, respectively. Adult skin fibroblasts from a healthy, 78 years old male Asian donor were purchased from CellResearch Corporation Pte Ltd and cultured in DMEM/10% FBS.

The culture medium PTT-4 consists of 90% (v/v) CMRL-1066 and 10% (v/v) FBS, while the medium PTTe-3 has the following composition:

| MCDB - 170/EpiLife | 200 ml/300 ml | |
|---|---|---|
| DMEM | 250 | ml |
| DMEM/F12 | 250 | ml |
| Fetal Bovine Serum | 1% | |
| Adenine | 0.05 to 0.1 | mM |
| Hydrocortisone | 0.1 to 0.5 | μM |
| Epidermal Growth Factor | 1 to 15 | ng/ml |
| T3 (3,3′,5-Triiodo-L-thyronine sodium | 0.1 to 5 | ng/ml. |
| salt) | | |
| Cholera toxin vibrio cholerae | 1 × 10⁻¹¹M to 1 × 10⁻¹⁰M | |
| Insulin | 1 to 7.5 | μg/ml |
| TGF-alpha | 1.0 to about 10 | ng/ml. |
| TGF-beta1 | 0.1 to 5 | ng/ml |

Somatic reprogramming was performed using the conditions established in Example 1 and was further in a feeder-independent manner. Log-phase cultures were harvested by dissociation with TrypLE Express (ThermoFisher Scientific) and 0.72 million cells were pelleted in a 1.5 ml centrifuge tube. The cell pellet was resuspended in 120 μL of Buffer R (Neon™ Transfection System 100 μL Kit, Thermo Fisher Scientific MPK10096). A cocktail containing 1.2 μg each of episomal vectors pCXLE-hOCT3/4-shp53-F, pCXLE-hSK, and pCXLE-hUL (Addgene plasmids #27077 (SEQ ID NO: 12), #27078 (SEQ ID NO: 13), #27080 (SEQ ID NO: 14), respectively) was added to the cells and mixed thoroughly (each vector was used in an amount of 1.67 μg (plasmid) DNA for a number of 1×10⁶ cells). The cell suspension was loaded into a 100 μL Neon® Tip and Neon electroporation was performed with the following parameters: adult skin fibroblasts—1,650 V, 10 ms, 3 pulses; CLEC—1350V, 30 ms, 2 pulses; CLMC—1600V, 20 ms, 1 pulse. Cells were immediately transferred into 6 ml of CLEC or CLMC medium containing 1 μM hydrocortisone (StemCell Technologies) and distributed equally into 3 wells of a Matrigel-coated 6-well plate. Two days later, the medium was switched to a 1:1 mixture of CLEC or CLMC medium and mTeSR1 supplemented with 1 μM hydrocortisone. On day 4 post-transfection, medium change was performed with the same medium. On day 6 post-transfection, medium was switched to complete mTeSR1 and hydrocortisone omitted from here on. Subsequently, medium change was performed every two days with mTeSR1. When iPS colonies reached about 1-2 mM in diameter (around Day 20 onwards), they were manually picked under bright field microscopy and each colony placed in a single well of Matrigel coated 24-well plate (Nunc). When cells in each well reached ˜50% confluence, they were detached with Dispase (StemCell Technologies) and transferred into a well of a Matrigel-coated 6-well plate. Subsequently, cells were passaged 1:3 by dissociation with 0.5 mM EDTA when they reached near confluence. Newly passaged cells were cultured overnight in medium containing 10 μM ROCK inhibitor Y-27632. In addition to mTeSR1, other commercial ES/iPS culture medium such as StemMACS™ iPS-Brew XF (Miltenyi Biotec) and TeSR-E8 (StemCell Technologies) have been used to maintain the iPS cultures.

### Protocol for Generating CLiPS:

1. Actively dividing CLEC or CLMC cultured in T-75 flasks in their maintenance medium PTTe-3 and PTT-4, respectively, are harvested by dissociation using TrypLE Express (ThermoFisher Scientific).
2. Cells are counted and 0.72 million cells are aliquoted into a microfuge tube and pelleted.
3. The cell pellet is resuspended in 120 μL of Buffer R (Neon™ Transfection System 100 μL Kit, Thermo Fisher Scientific MPK10096). A cocktail containing 1.2 μg each of pCXLE-hUL, pCXLE-hSK, and pCXLE-hOCT3/4-shp53-F is added and mixed thoroughly.
4. The cell suspension is loaded into a 100 μL Neon® Tip. Electroporation is performed with the following parameters for CLEC: 1350V, 30 ms, 2 pulses and the following parameters for CLMC: 1600V, 20 ms, 1 pulse.
5. Cells are immediately transferred into 4 ml of CLEC or CLMC medium (PTTe-3 and PTT-4, respectively) containing 1 μM hydrocortisone and then distributed into 3 wells of a Matrigel coated 6-well plate.
6. Two days after electroporation, the medium is changed to a 1:1 (v/v) mixture of CLEC or CLMC medium (PTT-e3 and PTT-4, respectively) and mTeSR1 supplemented with 1 μM hydrocortisone.
7. Four days after electroporation, a medium replacement is performed with the same 1:1 (v/v) media mixture.
8. Six days after electroporation, medium is changed to mTeSR1 only. Hydrocortisone is omitted from here on.
9. Medium replaced is performed every two days
10. iPS colonies may start appearing as early as 2 weeks after transfection. When iPS colonies reach about 0.5 mm to about 1 mm in diameter (around Day 20 onward), they are manually picked under bright field microscopy and each colony is placed in a single well of Matrigel coated 24-well plate (Nunc).
11. After colony picking, medium change for isolated colonies is performed daily.
12. When cells in each well occupy about 50% of the culture surface, they are detached with Dispase (StemCell Technologies) and transferred into a well of a Matrigel coated 6-well plate.
13. Subsequently, cells are passaged 1:3 by dissociation using 0.5 mM EDTA when they reach about 70%-80% confluence. Newly passaged cells are cultured overnight in medium containing 10 μM ROCK inhibitor Y-27632.

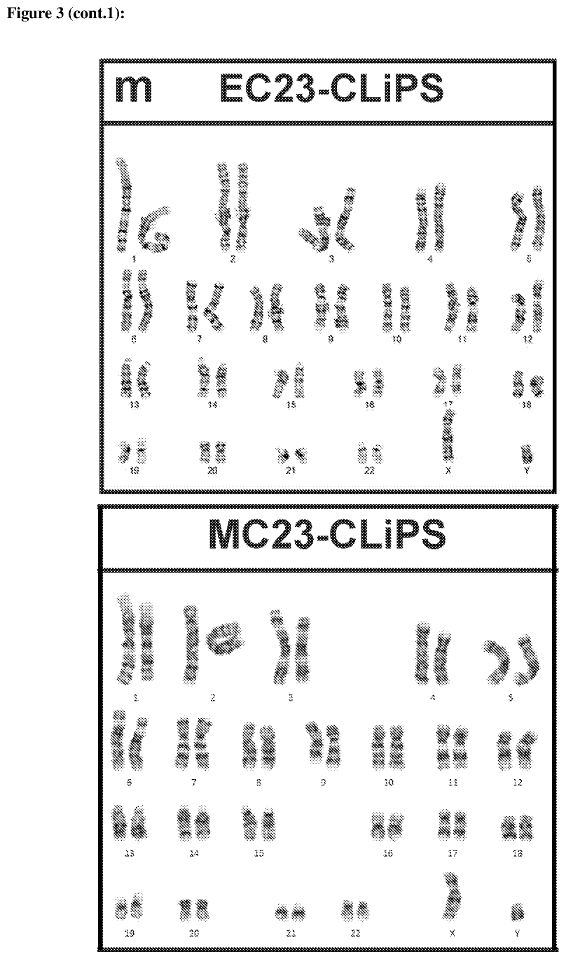

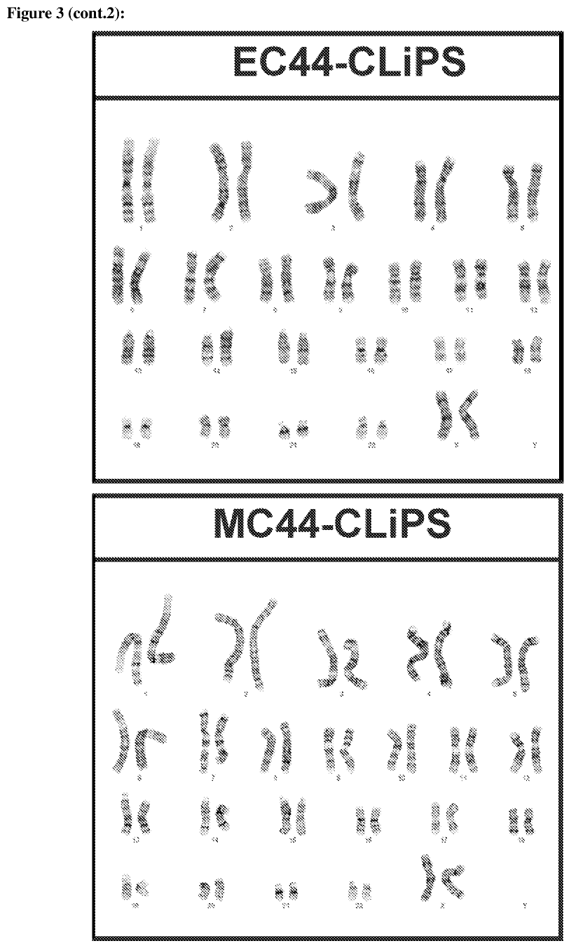

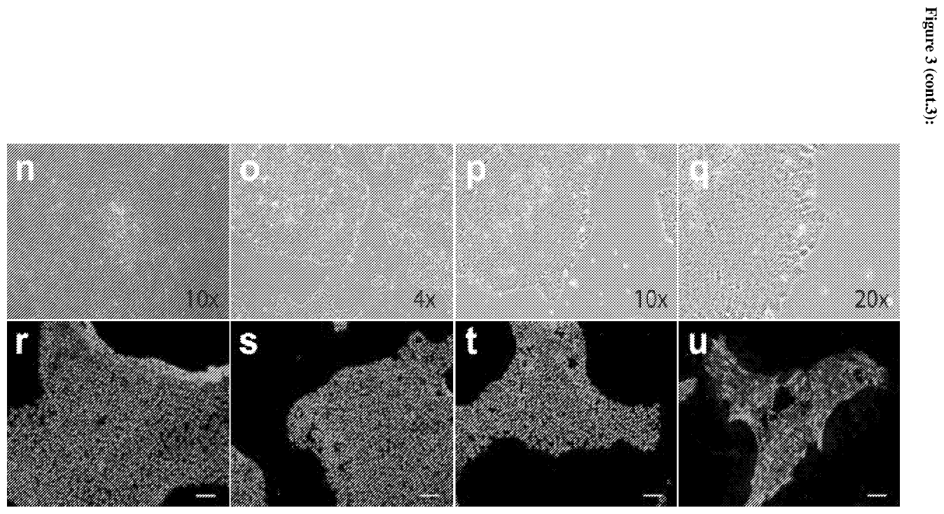

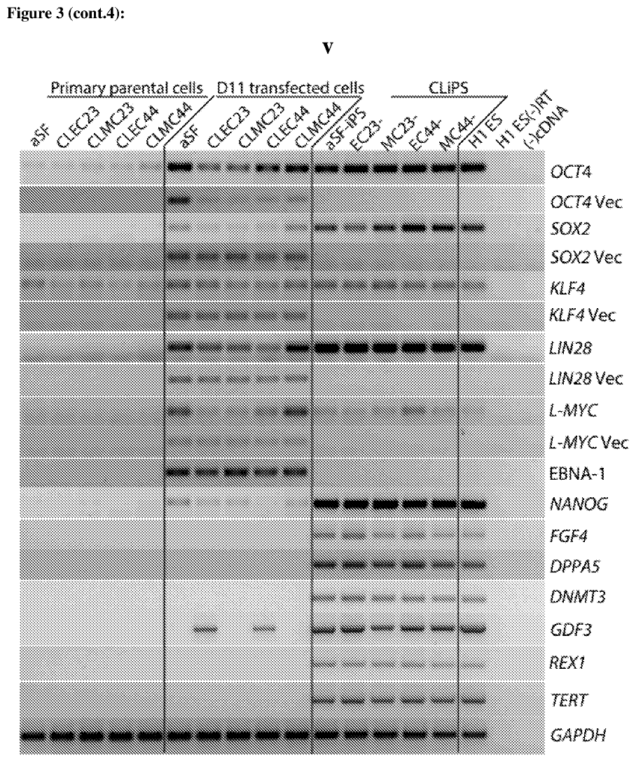

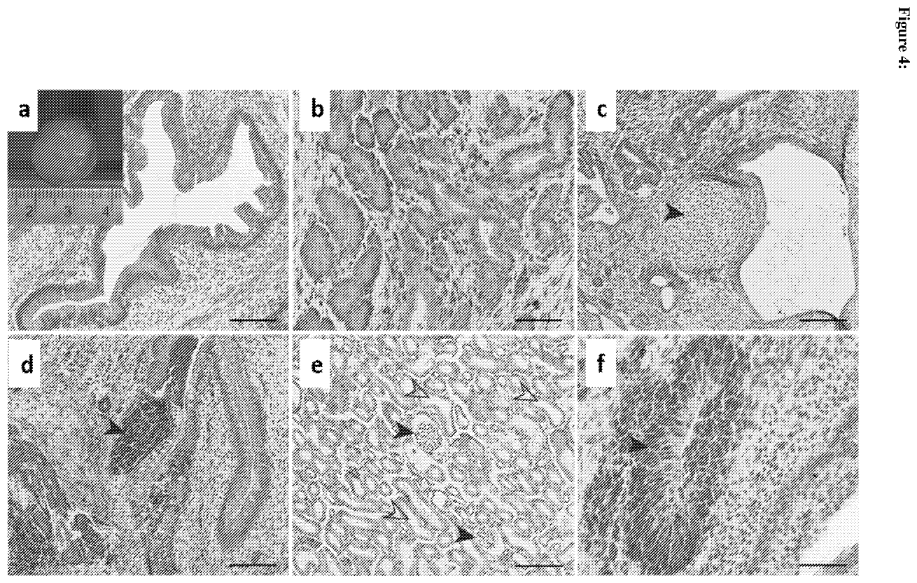

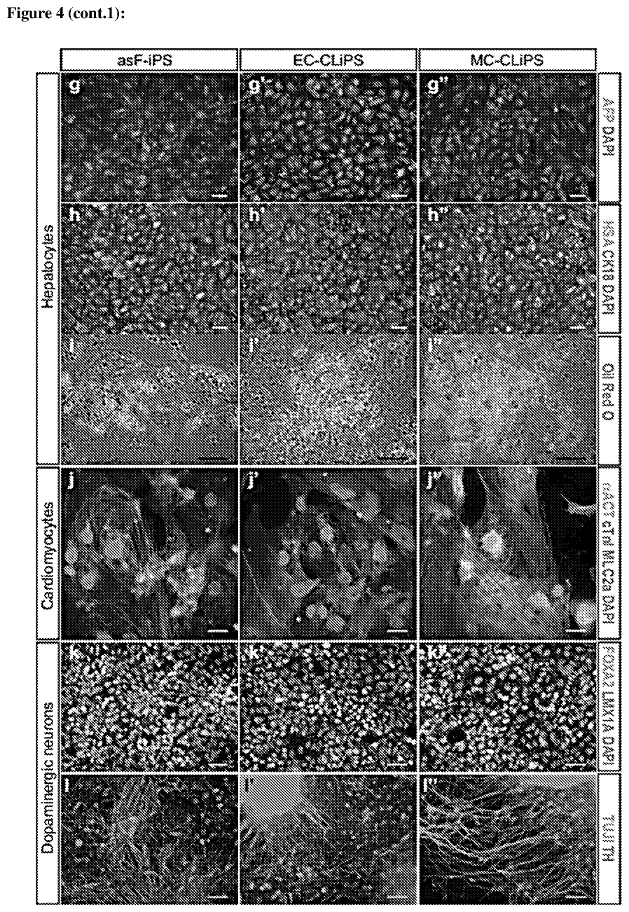

Following the protocol described above, small clusters of cells that look morphologically distinct from parental cells began to emerge starting at around Day 10. By Day 15, the cell clusters acquired defined edges (FIG. 3b) and discrete embryonic stem cell-like colonies appeared from Day 20 onwards (FIG. 3c and FIG. 3d). Colonies were picked when they reached 1-2 mm in diameter and expanded for characterization and storage. Expanded CLiPS exhibited cellular morphology indistinguishable from that of adult skin fibroblast-derived iPS or human embryonic stem cells (ES) with characteristic large nuclei and thin cytoplasm (FIG. 3e and FIG. 3f).

### Example 3: Derivation of cGMP-Compatible CLiPS (CLMSC-DTHN)

To provide proof-of-concept that CLiPS can be produced under conditions compatible with human therapeutic applications, iPS were generated from a cGMP-grade CLMC line designated CLMSC-DTHN using the protocol described in WO2018/067071 for the production of the mesenchymal stem population of which 99% of the stem cell express the markers CD73, CD90 and CD105 while not expressing the markers CD34, CD45 and HLA-DR) cGMP quality reagents wherever possible. The reprogramming protocol is the same protocol described for CLMC in Example 2 but Matrigel, an extracellular matrix substrate prepared from Engelbreth-Holm-Swarm (EHS) mouse sarcoma cells, was replaced with recombinant human laminin-511 E8 fragment (iMatrix-511 SILK, ReproCELL), which is a defined, animal- and xeno-free substrate for coating cell culture vessels. In addition, mTeSR1 used for reprogramming and subsequent maintenance of CLiPS clones was replaced with cGMP mTeSR™1 (StemCell Technologies).

Under the conditions as described herein, CLMSC-DTHN were reprogrammed with comparable dynamics and efficiencies as CLMC (data not shown). At 10 days post-transfection with reprogramming vectors, small clusters of cells with compact morphology can be observed (FIG. 3n). These clusters developed into isolatable colonies from Day 20 onwards. Expanded colonies displayed the characteristic cellular morphology of human pluripotent stem cells (FIG. 3n-q).

### Propagation and Cryopreservation of CLiPS

Sub-culturing of CLiPS (again using a media medium such as mTeSR1 or TeSR-E8 that is suitable for maintenance of iPS cells) is performed when cultures reached ˜90% confluence. Spent culture medium is aspirated off along with any overtly differentiated areas that may be present. Caution should be taken not to allow cells to be exposed to air for too long. The culture is rinsed once with prewarmed (37° C.) Dulbecco's Phosphate Buffered Saline (DPBS). Appropriate volume of rewarmed (37° C.) 0.5 mM EDTA solution is added to the culture according to the culture vessel—0.5 ml/well of a 24-well dish, 1 ml/well of a 6-well dish or 2 ml for a 6 cm dish. The culture is placed in an incubator at 37° C. for 5 min following which it is observed under a microscope. Cells should appear rounded but not detached from the surface. The duration of incubation at 37° C. varies with different CLiPS lines and may range from about 5-10 min. Incubation duration will be largely based on prior experience with each line. Following incubation, the EDTA solution is gentle aspirated off taking care not to dislodge the cells. Using a 1 ml pipettor, a medium such as mTeSR1 or TeSR-E8 containing ROCK inhibitor Y-27632 is dispensed directly onto the cells to dislodge them. The volume of medium used is dependent on the vessel size used—0.5 ml/well of a 24-well dish, 1 ml/well of a 6-well dish or 2 ml for a 6 cm dish. Gentle pipetting is repeated until most of the cells have been dislodged. The cell suspension is then transferred to a 15 ml Falcon tube. The culture vessel is rinsed with fresh medium and the rinse combined with the cell suspension in the Falcon tube. The cells in the tube are diluted to the appropriate volume for plating on new Matrigel-coated vessels. Split ratio may range from 1:3 to 1:10, depending on the density of the initial culture and also the growth rate of individual CLiPS lines.

For cryopreservation, cells are suspended in mTeSR1 or TeSR-E8 (or any other suitable culture medium) supplemented with 10% v/v of tissue culture-grade dimethyl sulfoxide (DMSO; e.g. Hybri-Max™, Sigma-Aldrich). The cell suspension is then aliquoted into appropriate numbers of cryovial. The density of cells per aliquot is dependent on the desired rate at which cell confluence is achieved upon thawing and culturing of the aliquot. Cryovials are then transferred to a slow freezing apparatus such as Mr. Frosty™ Freezing Container (Thermo Scientific) or CoolCell® Cell Freezing Containers (BioCision LLC) and placed overnight at −80° C. The next day, the cryovials are transferred to liquid nitrogen storage. It is not advisable to leave CLiPS aliquots at −80° C. for more than 24 hr. Several commercial freezing medium such as mFreSR™ (StemCell Technologies) and CryoStor® CS10 (Biolife Solutions) are also available for cryopreservation and may be used according to manufacturers' instructions.

### Example 4: Analysis of CLiPS Functionality

CLiPS functionality was determined by subjecting colony developing CLiPS to an immunofluorescent staining after electroporation. Thereby, the expression of pluripotent embryonic stem cell markers (OCT4, SOX2, KLF4, NANOG, SSEA-4, TRA-1-81) was analyzed. For this purpose, cells were fixed with 4% formaldehyde in phosphate buffer saline (PBS) for 15 min and subsequently washed 3 times for 5 min with PBS. For staining of intracellular or nuclear markers (OCT4, SOX2, KLF4, NANOG), cells were permeabilized with 0.1% Triton X-100 in PBS for 10 min, and blocked with FDB (5% FCS/1% NGS/1% BSA) for 1 h. For staining of surface markers (SSEA-4, TRA-1-81), the permeabilization step was omitted. Cells were incubated overnight with primary antibodies that were appropriately diluted with FDB at 4° C. followed by incubation with the appropriate fluorochrome-conjugated secondary antibodies at room temperature for 2 h. Stained samples were mounted in ProLong Diamond Antifade Mountant with DAPI (ThermoFisher Scientific).

Further, number and structure of the chromosomes within individual CLiPS lines were evaluated by performing a karyotype analysis and G-banding analysis, wherein the G-banding analysis was performed by the Cytogenetics Laboratory, KK Women's and Children's Hospital Pte. Ltd., Singapore.

Additionally, an RT-PCR analysis was performed to analyze the expression of reprogramming and pluripotent genes in primary parental cells, parental cells 11 days after vector transfection (D11 transfected cells) and CLiPS. For this purpose, total RNA was isolated from cell pellets using the RNeasy Mini or Plus Mini kits (Qiagen). Two μg of total RNA was treated with DNase I and used for cDNA synthesis with the RevertAid H Minus First Strand cDNA Synthesis Kit (Fermentas, Thermo Fisher Scientific). PCR reactions were set up as follows: 0.5 μl cDNA, 5 μl 2× MyTaq HS Mix (Bioline), 0.2 μl forward primer (10 μM), 0.2 μl reverse primer (10 μM), 4.2 μl PCR water. Thermal cycling was performed in an MJ Mini Thermal Cycler (Bio-Rad) with the following conditions: 1×95° C. 1 min, 30× (95° C. 15 s, Tm 15 s, 72° C. 15 s), 72° C. 1 min. Primer sequences and annealing temperature used (Tm) are provided in Table 2 below.

A qualitative expression analysis was performed by an agarose gel analysis, wherein the samples were loaded on a 2% agarose gel incorporated with SYBR Safe DNA stain (Thermo Fisher Scientific) in 1× TAE buffer and electrophoresed at 80V for 30 min. The gel images were captured using a ChemiDoc Imaging System (Bio-Rad).

TABLE 2
Primer sequences

| | SEQ | | | Amplified |
|---|---|---|---|---|
| Oligo | ID | | | fragment |
| Name | No | Sequence (5′ → 3′) | Tm | (bp) |
| hNanogF | 15 | AAGGTCCCGGTCAAGAAACAG | 55 | 237 |
| hNanogR | 16 | CTTCTGCGTCACACCATTGC | 55 | |
| hKlf4F | 17 | CCCACATGAAGCGACTTCCC | 55 | 169 |
| hKlf4R | 18 | AGGTCCAGGAGATCGTTGAAC | 55 | |
| hSox2F | 19 | TGGACAGTTACGCGCACATG | 55 | 214 |
| hSox2R | 20 | GAGTAGGACATGCTGTAGGTG | | |
| hOct4F | 21 | TGCGGCCCTTGCTGCAGAAG | 60 | 201 |
| hOct4R | 22 | GCTGCTGGGCGATGTGGCTG | | |
| Oct4VecF | 23 | ATGCATTCAAACTGAGGTAAGG | 55 | 127 |
| pCXLE-R2 | 24 | TAGCGTAAAAGGAGCAACATAG | | |
| Lin28F/ | 25 | CCATATGGTAGCCTCATGTCC | 55 | 126 |
| VecF | | | | |
| hLin28R | 26 | TCAATTCTGTGCCTCCGGGAG | | |
| Klf4VecF | 27 | ACCACCTCGCCTTACACATGAAG | 55 | 156 |
| pCXLE-R2 | 28 | TAGCGTAAAAGGAGCAACATAG | | |
| L-mycVecF | 29 | GGCTGAGAAGAGGATGGCTAC | 55 | 124 |
| pCXLE-2AR | 30 | AGTTTGTTTGACAGGAGCGAC | | |
| Sox2VecF | 31 | TCACATGTCCCAGCACTACC | 55 | 112 |
| pCXLE-2AR | 32 | AGTTTGTTTGACAGGAGCGAC | | |
| hL-mycF | 33 | AACCCAAGACCCAGGCCTGC | 60 | 135 |
| hL-mycR | 34 | GGTCTGCTCGCACCGTGATG | | |
| EBNA-1F | 35 | GAAATGGCCTAGGAGAGAAG | 55 | 214 |
| EBNA-1R | 36 | CAGCCAATGCAACTTGGACG | | |
| hGDF3F | 37 | CTTATGCTACGTAAAGGAGCTGGG | 60 | 633 |
| hGDF3R | 38 | TTGTGCCAACCCAGGTCCCGGAAG | | |
| hREX1F | 39 | TATCAGATCCTAAACAGCTCGCAG | 55 | 308 |
| hREX1R | 40 | CGTACGCAAATTAAAGTCCAGAG | | |
| hFGF4F | 41 | ACTACAACGCCTACGAGTCCTAC | 60 | 372 |
| hFGF4R | 42 | GTTGCACCAGAAAAGTCAGAGTTG | | |
| hDPPA5F | 43 | ATATCCCGCCGTGGGTGAAAGTTC | 60 | 243 |
| hDPPA5R | 44 | ACTCAGCCATGGACTGGAGCATCC | | |
| hTERTF | 45 | CCTGCTCAAGCTGACTCGACACCG | 65 | 446 |
| | | TG | | |
| hTERTR | 46 | GGAAAAGCTGGCCCTGGGGTGGAG | | |
| | | C | | |
| hDNMT3BF | 47 | TGCTGCTCACAGGGCCCGATAC | 60 | 242 |
| hDNMT3BR | 48 | TCCTTTCGAGCTCAGTGCACCAC | | |
| hGAPDHF | 49 | CTGGCGCTGAGTACGTCGTGG | 60 | 200 |
| hGAPDHR | 50 | GCAGTTGGTGGTGCAGGAGGC | | |

The results show that CLiPS showed robust expression of the human embryonic stem cell (hES) markers KLF4, NANOG, OCT4, SOX2, SSEA4, and TRA-1-60 as demonstrated by antibody staining (FIG. 3g-l). G-banding analysis showed that CLiPS maintained a normal karyotype up to 17 passages from colony picking (FIG. 3m). RT-PCR analysis of gene expression in parental cells, Day 11 post-transfected cells and expanded iPS clones revealed that activation of endogenous OCT4, SOX2, KLF4, LIN28 and L-MYC genes has replaced the roles of vector-driven expression of these genes for the maintenance of pluripotency in fully reprogrammed CLiPS (FIG. 3v). The induction of endogenous NANOG loci, a crucial gene for somatic reprogramming, was evident at Day 11 post-transfection. The absence of detectable levels of EBNA-1 transcripts in CLiPS clones suggests that plasmid vectors have been lost from these cells. The expression of additional hES-specific genes GDF3, DPPA5, DNMT3, FGF4, and REX-1 in CLiPS further confirms their hES-like molecular phenotype. TERT, which encodes the catalytic reverse transcriptase subunit of telomerase that is essential for the regulation of self-renewal and the maintenance of pluripotency, is expressed in CLiPS at levels identical to that in H1 hES.

### Example 5: Expression Analysis of Pluripotent Embryonic Stem Cell Markers in CLiPS-DTHN

To analyze the expression of pluripotent embryonic stem cell markers (Oct4, Sox2, Klf4, Nanog) indicating pluripotency, developing CLMSC-DTHN were subjected to an immunofluorescent staining after electroporation. The immunofluorescent staining protocol was the same protocol described for CLiPS in Example 4.

The results show that CLMSC-DTHN express the pluripotent stem cell markers NANOG, OCT4, SOX2 and TRA-1-81 (FIG. 3r-u) at levels indistinguishable from their non-GMP counterparts. Thus, CLMSC-DTHN may provide the same embryonic properties non-GMP-derived CLiPS entail.

### Example 6: Determining CLiPS Pluripotency

The pluripotency of CLiPS and aSF-iPS was evaluated by teratoma formation assay in NOD-SCID mice. For this purpose, 1×106 CLiPS cells were pelleted, resuspended in 0.1 ml of ice-cold Matrigel and injected into the dorsal flank of 6-8 week old NOD/MrkBomTac-Prkd^{scid} mice. Mice were sacrificed after 3 month later and teratomas harvested for histological analysis, wherein Paraffin wax sectioning and hematoxylin and eosin staining were performed using standard techniques.

The results show that palpable tumors developed in some mice beginning from 1 month following subcutaneous injection of iPS into the dorsal flank of mice. Histological analysis of the teratomas isolated 3 months after injection revealed spontaneous differentiation of CLiPS into tissues of endodermal, mesodermal and ectodermal lineages (FIG. 4a-f).

### Example 7: Differentiation of CLiPS into Dopaminergic Neurons

As an important prerequisite for potential future therapeutic application of CLiPS, it is necessary to demonstrate their capability to differentiate into specific tissue type under defined in vitro conditions. For dopaminergic neuronal differentiation, the midbrain floor plate induction protocol described in Kriks, S., et al, Nature, 2011. 480(7378): p. 547-51 was used for the differentiation of iPS into dopaminergic neuroprogenitors and neurons. Briefly, iPS were plated at a density of 3.5-4.0×10⁴ cells per cm² on Matrigel (Corning) coated dishes and cultured for 5 days in knockout serum replacement medium (KSR) containing Knock-Out DMEM, 15% knockout serum replacement, 1× GlutaMAX and 10 mM β-mercaptoethanol. From Day5, KSR medium was transitioned stepwise to N2 medium as described in Tomishima “Midbrain dopamine neurons from hESCs.” 2012 Jun. 10. In: StemBook. Cambridge (MA): Harvard Stem Cell Institute; 2008. Available from: https://www.ncbi.nlm.nih.gov/books/NBK133274/doi: 10.3824/stembook.1.70.1. On day 11, media was changed to NB27 medium composed of Neurobasal medium, 2% B27 minus vitamin A and 1× GlutaMAX and supplemented with CHIR (until day 13), BDNF (brain-derived neurotrophic factor, 20 ng/ml; Miltenyi), ascorbic acid (0.2 mM, Sigma), GDNF (glial cell line-derived neurotrophic factor, 20 ng/ml; Miltenyi), TGFβ3 (transforming growth factor type β3, 1 ng/ml; R&D), dibutyryl cAMP (0.5 mM; Santa Cruz Biotechnology), and DAPT (10 nM; Tocris) for 9 days. On day 20, cells were dissociated using Accutase (Gibco) and replated at high cell density (3-4×10⁵ cells per cm²) on dishes pre-coated with poly-L-ornithine (PLO; 15 mg/ml)/laminin (1 μg/ml)/fibronectin (2 μg/ml) in NB27 medium supplemented with 10 μM ROCK inhibitor Y-27632. Cultures were maintained in NB27 medium with medium replacement every other day until the desired endpoint. Differentiated cells were analysed for expression of cell specific markers at this stage. For this purpose, cryosectioning was performed, wherein slides containing the sections were dehydrated by incubation at 37° C. for 30 min, cooled to room temperature and washed 3 times with TBST. Section permeabilization, blocking, antibody staining and mounting were performed as described in Example 4. Primary antibodies from the same host species were used, a fluorochrome-conjugated monovalent antibody (Jackson ImmunoResearch) was used to saturate the first primary antibody before sequential incubation with the second primary antibody and conjugated secondary antibody.

The results show that dopaminergic neurons were obtained from CLiPS and asF5-iPS using this protocol. The antibody staining revealed that almost 90% of the cells coexpressed the floor-plate marker FOXA2 and the roof plate marker LMX1A (FIG. 4k, k′, k″), a definitive hallmark of midbrain DA neuron precursors. Further differentiation yielded abundant mature neurons as indicated by TUJ1 staining, out of which approximately 30-50% coexpressed the dopaminergic marker Tyrosine Hydroxylase (TH) (FIG. 4l, l′, l″). Electrophysiological analysis of CLiPS-derived neurons at Day 45 of differentiation demonstrated that the cells exhibited mature functional properties, with trains of action potential displaying the voltage sag response characteristic of mature mesencephalic DA neurons upon injection of hyperpolarizing currents (FIG. 4m).

### Example 8: Differentiation of CLiPS into Hepatocytes

As an important prerequisite for potential future therapeutic application of CLiPS, it is necessary to demonstrate their capability to differentiate into a desired target cell type, or a specific tissue type under defined in vitro conditions. For hepatic differentiation, a protocol that was originally developed for the differentiation of human embryonic cells (ES) on mouse feeder layer (Medine, C. N., et al., J Vis Exp, 2011(56): p. e2969) was adapted for CLiPS and asF-iPS differentiation in mTeSR1 on Matrigel. A modification is that when iPS cultures reached 20-30% confluency, DMSO was supplemented to 2% and incubated for 24 h. When cultures reached confluency of ˜30%-60%, definitive endoderm formation was induced by replacing the mTeSR1 with priming medium (RPMI 1640-B27 supplemented with 100 ng/mL Activin A and 50 ng/mL Wnt3a). Cultures were maintained in priming medium for 3 days with medium change every 24 h. After 72 h in priming medium, the medium was switched to SR-DMSO (80% KO-DMEM, 20% KO-SR, 0.5% L-glutamine, 1% non-essential amino acids, 0.1 mM β-Mercaptoethanol and 1% DMSO) for 5 days with medium change every 48 hours). At day 8, cultures were switched to L-15 maturation and maintenance medium (Leibovitz L-15 medium, 8.3% tryptose phosphate broth, 8.3% heat inactivated FBS, 1 μM hydrocortisone 21-hemisuccinate, 1 μM Insulin (bovine pancreas), 1% L-Glutamine, 0.2% ascorbic acid) supplemented with 10 ng/mL hHGF and 20 ng/mL OSM for 9 days (changing medium every 48 hours). Differentiated cells were again analysed for expression of cell specific markers at this stage. For this purpose, cryosectioning was performed as described in Example 7.

The results show that hepatocyte-like cells were obtained from CLiPS and asF5-iPS using this protocol. The antibody staining revealed the expression of the hepatocyte markers alpha-fetoprotein (AFP; FIG. 4g, g′, g″), Cytokeratin 18 (CK18) and Human Serum Albumin (HSA; FIG. 4h, h′, h″) after 17 days of differentiation. A majority of the differentiated cells exhibited a polygonal shape characteristic of hepatocytes. In addition, staining with Oil Red O showed abundant lipid droplet accumulation in the cells, a hallmark of cultured hepatocytes (FIG. 4i, i′, i″).

### Example 9: Differentiation of CLiPS into Cardiomyocytes

As an important prerequisite for potential future therapeutic application of CLiPS, it is necessary to demonstrate their capability to differentiate into specific tissue type under defined in vitro conditions. For cardiomyocyte differentiation, the protocol for cardiomyocyte differentiation of iPS was adapted from the protocol described in Lian, X., et al., Proc Natl Acad Sci USA, 2012. 109(27), p. E1848-57. iPS maintained on Matrigel in mTeSR1 were dissociated into single cells with StemPro Accutase (Thermo Fisher Scientific) at 37° C. for 5 min and then seed onto a Matrigel-coated cell-culture dish at 1×10⁵-2×10⁵ cell/cm² (5×10⁵ cells per 24-well) in mTeSR1 supplemented with 5 μM ROCK inhibitor (Y-27632; Stemgent) for 24 h. In a modification, when cells reached ˜80% confluency, medium was switched to mTeSR1 supplemented with 2% DMSO. When cells reached confluence, they were treated with CHIR99021 in RPMI/B27-insulin for 24 h. In another modification, the concentration of CHIR99021 was lowered to 5 μM from the original 1 μM at this stage. The next day, the medium was changed to RPMI/2% B27 without insulin. Two days later, half of the old medium was combined with an equal volume of fresh medium containing 1 μM IWP2 (Tocris). The remaining medium in the wells was discarded and the mixture added to the cultures. Two days later, medium was switched to RPMI/2% B27 without insulin only. After 48 hr, cultures were maintained in RPMI/2% B27 with medium change every 3 days until the desired endpoint. Beating cardiomyocytes were fixed and stained for cell specific markers as described in Example 7.

The results show that cardiomyocytes were obtained from CLiPS and asF5-iPS using this protocol. The antibody staining revealed the expression of the spontaneously contracting cardiomyocytes were observed beginning from 8 days of differentiation. Immunofluorescent antibody staining for the functional cardiac markers Myosin regulatory light chain 2a (MLC2a), cardiac troponin I (cTnI) and alpha-actinin (αACT) revealed sarcomeric structures within differentiated cardiomyocytes (FIG. 4j, j′, j″). No noticeable difference in differentiation efficiency was observed between.

### Example 10: Differentiation of CLiPS into Oligodendrocytes

To further demonstrate the ability of the induced pluripotent stem cells of the invention to differentiate into a given target cell type, CLiPS were differentiated into oligodendrocytes. Oligodendrocyte differentiation of CLiPS and asF-iPS was performed according to the protocol of Douvaras, P. and V. Fossati, Nat Protoc, 2015. 10(8): p. 1143-54. Further, cryosectioning was performed as described in Example 7 to analyse the expression of cell specific markers.

At Day75 of differentiation, clusters of Olig2-positive oligodendrocyte precursor cells (OPCs; FIG. 4n) and also 04-positive late OPCs were obtained (FIG. 4o).

### Example 11: Immunogenicity Analysis

To gain some insight into the immunogenicity of CLiPS and their neural derivatives, the expression of a panel of immunogenicity related markers by these cells was assessed by flow cytometric analysis. For this purpose, primary cells and Day25 differentiated DA NPCs were harvested by dissociation with TrypLE Express while iPS cultures were harvested by dissociation with 0.5 mM EDTA. Cells were resuspended in 1× Ca²⁺- and Mg²⁺-free DPBS containing 0.1% bovine serum albumin (BSA) to 5 million cells/ml. 100 μl of cells was stained with the appropriate conjugated antibodies or their isotype controls in the dark for 30 min on ice. For HLA-E and HLA-G staining, cells were permeabilized with BD Phosflow Perm/Wash Buffer I (BD Biosciences) according to manufacturer's instructions prior to staining. Following staining, cells were washed 2× in 1× Ca²⁺- and Mg²⁺-free DPBS/5 mM EDTA, fixed with 1% paraformaldehyde for 1 hr in the dark and then were washed 2× in 1× Ca²⁺- and Mg²⁺-free DPBS/5 mM EDTA. Cells were resuspended in 0.5 ml 1× Ca²⁺- and Mg²⁺-free DPBS/5 mM EDTA and analysed on a flow cytometer. Stained primary cells and iPS were analysed on a FACSCalibur while stained dopaminergic neuronal progenitor cells (NPCs) were analysed on a FACSCanto II instrument (both from BD Biosciences). Data was analysed using FlowJo software package (FlowJo LLC). Antibodies used are listed in Table 3.

TABLE 3
Antibodies used for flow cytometry

| | | | | BioLegend |
|---|---|---|---|---|
| Antigen | Isotype | Conjugate | Dilution | Cat. No. |
| CD40 | Mouse IgG1, κ | FITC | 1:20 | 303604 |
| CD80 | Mouse IgG1, κ | FITC | 1:20 | 305206 |
| CD86 | Mouse IgG2b, κ | Alexa | 1:20 | 305414 |
| | | Fluor 488 | | |
| HLA-A, B, C | Mouse IgG2a, κ | PE | 1:20 | 311406 |
| HLA-DR | Mouse IgG2a, κ | PE | 1:20 | 307606 |
| HLA-E | Mouse IgG1, κ | PE | 1:20 | 342604 |
| HLA-G | Mouse IgG2a, κ | APC | 1:20 | 335910 |
| NCAM/CD56 | Mouse IgG1, κ | APC/Cy7 | 1:20 | 318332 |
| Isotype control | Mouse IgG1, κ | FITC | 1:20 | 400108 |
| Isotype control | Mouse IgG2b, κ | Alexa | 1:20 | 400329 |
| | | Fluor 488 | | |
| Isotype control | Mouse IgG1, κ | PE | 1:20 | 400112 |
| Isotype control | Mouse IgG2a, κ | PE | 1:20 | 404212 |
| Isotype control | Mouse IgG1, κ | APC | 1:20 | 400120 |
| Isotype control | Mouse IgG2a, κ | APC | 1:20 | 400222 |
| Isotype control | Mouse IgG1, κ | APC/Cy7 | 1:20 | 400128 |

MHC Class I HLA-A, -B and -C and MHC Class II HLA-DR molecules are known to be important for alloimmune response. The results show that HLA-ABC was expressed across all iPS samples but a markedly reduced level was observed for EC23-CLiPS (FIG. 6a). HLA-DR expression was absent in all iPS samples (FIG. 6b), consistent with previous reports of negligible HLA-II expression in iPS (Saljö, K., et al., Sci Rep, 2017. 7(1): p. 13072 and Chen, H. F., et al., Cell Transplant, 2015. 24(5): p. 845-64). T cell co-stimulatory molecules CD40, CD80, and CD86 play an important role in activating T cells during alloimmune response. Of the three molecules examined, only CD40 was expressed on iPS, with the lowest level expressed by asF-iPS and the highest level expressed by MC23-CLiPS compared to the rest (FIG. 6a). As the tolerogenic HLA-E and HLA-G have been reported to be expressed on CLMC (Deuse, T., et al., Cell Transplant, 2011. 20(5): p. 655-67) and CLEC (Zhou, Y., et al., Cell Transplant, 2011. 20(11-12): p. 1827-41), the expression of these antigens by CLiPS was also investigated. Analysis of permeabilized cells revealed only marginal expression of HLA-E in MC23-CLiPS and EC44-CLiPS, and below detectable levels in other samples. Next, the expression profiling of the entire panel of markers on Day 25 DA differentiation cultures was repeated. Analysis was performed on neural cells populations gated on positive staining for NCAM. NCAM+ fractions exceeded 97% for all samples, with asF-iPS and EC23-CLiPS showing comparable differentiation efficiencies of 99.5% (FIG. 6b). HLA-ABC was expressed by all NPC samples but at generally lower levels compared to their parental iPS (FIG. 6c). EC23-CLiPS-derived NPCs expressed the lowest level of HLA-ABC amongst the samples, mirroring the trend displayed by its parental iPS. The level of HLA-ABC expression on MC23-CLiPS was reduced upon its differentiation to NPCs. CD40 expression was downregulated across all NPC samples, with only EC23-iPS- and EC44-iPS-derived NPCs displaying slight expression. HLA-E expression was absent in all NPC samples but slight upregulation of HLA-G was observed in asF-iPS- and EC23-iPS-derived NPCs. These results indicate reduced immunogenity in CLiPS.

### Example 12: Transplantation of CLiPS-Derived Dopaminergic Neurons in Fully Immunocompetent Mice Models of Parkinson's Disease

Previous studies have shown that dopaminergic neurons generated from human embryonic stem cells and iPS using various protocols can engraft in rodent (Kriks, S., et al, Nature, 2011. 480(7378): p. 547-51; Hargus, G., et al., Proc Natl Acad Sci USA, 2010. 107(36): p. 15921-6; Doi, D., et al., Stem Cell Reports, 2014. 2(3): p. 337-50; Grealish, S., et al., Cell Stem Cell, 2014. 15(5): p. 653-65; Kirkeby, A., et al., Cell Rep, 2012. 1(6): p. 703-14; Qiu, L., et al., Stem Cells Transl Med, 2017. 6(9): p. 1803-1814; Rhee, Y. H., et al., J Clin Invest, 2011. 121(6): p. 2326-35; Samata, B., et al., Nat Commun, 2016. 7: p. 13097; Wakeman, D. R., et al., Stem Cell Reports, 2017. 9(1): p. 149-161) and non-human primate (Kriks, S., et al, Nature, 2011. 480(7378): p. 547-51; Hargus, G., et al., Proc Natl Acad Sci USA, 2010. 107(36): p. 15921-6; Wakeman, D. R., et al., Stem Cell Reports, 2017. 9(1): p. 149-161; Daadi, M. M., et al., PLoS One, 2012. 7(7): p. e41120; Kikuchi, T., et al., Nature, 2017. 548(7669): p. 592-596) models of Parkinson's Disease (PD). In all these studies, animals were either immunocompromised or otherwise immunosuppressed pharmacologically to prevent graft rejection. The need for immunocompromised or immunosuppressed animals was only obviated when transplantation was performed using either autologous (Morizane, A., et al., Stem Cell Reports, 2013. 1(4): p. 283-92; 4. Hallett, P. J., et al., Cell Stem Cell, 2015. 16(3): p. 269-74; Wang, S., et al., Cell Discov, 2015. 1: p. 15012; Emborg, M. E., et al Cell Rep, 2013. 3(3): p. 646-50; Sundberg, M., et al., Stem Cells, 2013. 31(8): p. 1548-62) or MHC-matched allogenic (Morizane, A., et al., 2017. 8(1): p. 385) iPS-derived cells.

To demonstrate the engraftability of CLiPS-derived DA NPCs differentiated using the method of the present invention, transplanted Day 25 NPCs differentiated from asF-iPS, EC23-CLiPS and MC23-CLiPS were transplanted into immunocompromised NOD-SCID mice (n=3). In this context, it is noted that all animal experiments were conducted according to protocols approved by the Institutional Animal Care and Use Committee (IACUC) of the National Neuroscience Institute (NNI), Singapore.

To test immunogenicity of CLiPS-derived DA NPCs, a transplantation needs to be performed on PD models. For this purpose, 6-hydroxydopamine (6-OHDA) unilateral lesion mouse models were generated. The unilateral 6-OHDA lesion is an established method for rodents and comprises the injection of 6-OHDA in the rodent brain causing motoric dysfunctions characterized by rotational asymmetry with the degree (Bagga, V., Dunnett, S. B. and Fricker, R. A. (2015) Behavioural Brain Research. Elsevier B. V., 288, pp. 107-117). In the present invention, 6-OHDA lesions were induced in NOD/MrkBomTac-Prkdc^{scid} mice (4 weeks old) purchased from InVivos Pte Ltd and maintained under SPF conditions at the Animal Research Facility, NNI and male C57BL/6NTac mice (6-8 weeks old) purchased from InVivos Pte Ltd., wherein the mice used for this experiment were fully immunocompetent and no immunosuppression was administered prior to or following transplantation.

To generate the mouse PD model, 7.5 μg of 6-OHDA (Sigma, Merck-Millipore; dissolved at 2.5 mg/ml in 0.9% NaCl containing 0.2% ascorbic acid) was delivered into the left striatum by stereotaxic injection at the following coordinates: anterior-posterior (AP)+0.5 mm; medial-lateral (ML) −1.8 mm from Bregma; dorsal-ventral (DV) −3.0 mm from cranium. After two weeks of acclimatization, transplantation of 3 NPCs samples (i.e. NPCs derived from asF-iPS, EC23-CLiPS and MC23-CLiPS) were transplanted into the striatum of immunocompetent, 6-OHDA lesioned C57BL/6 mice by stereotaxic injection was performed on mice models considered as successfully lesioned.

To determine suitable models for transplantation, apomorphine-induced rotations were scored and mice displaying >6 rotations per min were used for transplantation. For transplantation, Day25 dopaminergic progenitor cells were harvested by dissociation and resuspended to ˜1.25×10⁵ cells/μl in HBSS supplemented with 10 ng/mL of BDNF, 10 ng/mL of GDNF. Two μL of cell suspension was injected into lesioned mice at the following coordinates: AP +0.5 mm; ML −2.0 mm, and DV −2.8 mm from skull. To assess whether transplanted NPCs can integrate and mediate functional benefits in lesioned animals, rotation asymmetry tests were performed at 2-weekly intervals. Rotation assays were performed every 2 weeks up to 9 months, wherein mice were injected intraperitoneally with 0.05 mg/kg of apomorphine dissolved in 0.9% NaCl containing 0.1% w/v ascorbic acid. Rotations were recorded using a digital camera and counted manually. Batches of animals were sacrificed at 1, 6 and 9 months after transplantation by terminal anesthesia.

Six months after transplantation, NPC-transplanted, sham-injected and non-manipulated mice were evaluated for striatal dopamine transporter (DAT) activity by positron-emission tomography (PET) imaging using the radioligand (2-[18F]Fluoroethyl 8-[(2E)-3-iodoprop-2-en-1-yl]-3-(4-methylphenyl)-8-azabicyclo[3.2.1]octane-2-carboxylate) ([18F]FE-PE2I). Animals were fasted for 3 hours prior to the imaging sessions. Animals were kept warm during scanning with integrated hot-air channels from the imaging bed. The respiratory rate and temperature were monitored throughout the entire scan session to ensure adequate level of anaesthesia. Mice were imaged using the nanoScan PET/MRI scanner (Mediso Ltd., Hungary) at the SingHealth Experimental Medicine Centre (SEMC). This scanner is equipped with 12 detector modules with an axial field of view (FOV) of 94 mm and a transaxial FOV of 94 mm or 120 mm diameter in 1:3 and 1:5 coincidence modes, respectively. Animals were placed head first in a prone position and a 3D dynamic PET scan was performed for 62 min with frames of increasing durations i.e. 4 at 10 sec, 4 at 20 sec, 3 at 1 min, 7 at 3 min and 6 at 6 min) following intravenous injection of 3.57-10.61 MBq of [18F]FE-PE2I in a maximum volume of 0.1 ml via the tail vein. [18F]PE-PE2I was synthesized at the Singapore Radiopharmaceuticals Pte Ltd. MRI images were used for the attenuation correction of the PET scans and as structural reference for the PET images in data analysis. Thus, T1-weighted MRI images were acquired using the MRI component of the nanoScan PET/MRI scanner. The integrated mouse head coil covers the entire brain during the MRI scan. Slices of 0.6 mm were obtained using a 3D GRE EXT sequence: 64-mm square FOV, 128×128 matrix, 20-ms repetition time (TR), 2.3-ms echo time (TE), 25 degree flip angle. All image and kinetic analyses for the [18F]FE-PE2I PET images were performed using PMOD (version 3.5; PMOD Technologies). All PET images were first automatically registered to the MRI images using the FUSION tool in PMOD. The MRI images were then manually registered to the T2-weighted mouse template (M. Mirrione, C57BL/6J mice; Ma, Y., et al., Neuroscience, 2005. 135(4): p. 1203-15; Mirrione, M. M., et al., Neuroimage, 2007. 38(1): p. 34-42), which contains a volume of interest (VOI) template with 20 regions. The accuracy of the manual registration was accessed and verified by two different persons. Finally, the combined transformation matrix was applied to transform the PET images to the MRI mouse template. VOIs for left and right striatum and cerebellum were used for the analysis. In order to reduce the errors from misregistration and misdefinition (He, B. and E. C. Frey, Phys Med Biol, 2010. 55(12): p. 3535-44), 3D erosion with one voxel was applied to the obtained VOIs. [18F]FE-PE2I binding was quantified using the non-invasive reference tissue models, since they are equally accurate as compared to the kinetic analyses with the arterial input function (Varrone, A., et al., Nucl Med Biol, 2012. 39(2): p. 295-303). The binding potential (BPnd) values were calculated using the simplified reference tissue model (SRTM) (Lammertsma, A. A. and S. P. Hume, 1996. 4(3 Pt 1): p. 153-8) with the cerebellum as the reference. The regional time activity curves (TACs) were also extracted from the VOIs of striatum and cerebellum. Anesthesia was induced with 5% isoflurane in 100% O₂ and maintained with 1.5-2% isofluorane during the imaging.

Brain sections of mice were analyzed for the presence of microglia/macrophages, as these cells are known to play important roles in allograft and xenograft rejection in the CNS (Hoornaert, C. J., et al., Stem Cells Transl Med, 2017. 6(5): p. 1434-1441), For this purpose, an immunostaining for the microglia/macrophages-specific marker Iba1 was performed after transcardial perfusion with 4% PFA. For this purpose, PFA perfused brains were post-fixed overnight in 4% PFA followed by equilibration in 15% and 30% w/v sucrose solution in PBS until they settled to the bottom of the tubes. Brains were embedded in OCT freezing medium and 18 μm sections were cut on a CM3050 S cryostat (Leica Biosystems) and collected on BOND Plus Slides (Leica Microsystems).

The results show that hNCAM+/TH+ neurons were present in all 3 groups 1-month post-transplantation (FIG. 7a-c), suggesting that the NPCs are capable of differentiating into mature neurons and surviving in the host environment. However, no signs of engraftment were evident in the asF-iPS (FIG. 7h) or MC23-iPS (data not shown) groups. hNCAM/TH+ fibers can be seen extending from neurons in the graft core of the EC23-CLiPS group along axonal tracts of the corpus callosum (FIG. 7d and FIG. 7e). The immunostaining for the microglia/macrophages-specific marker Iba1 revealed an abundance of microglia/macrophage in the injected hemisphere compared to the non-injected hemisphere (FIG. 7i and FIG. 7j). Microglia/macrophages that infiltrated into the core of the graft displayed an amoeboid morphology characteristic of activated microglia compared to those at the periphery of the grafts which showed a ramified morphology typical of quiescent cells. In addition, infiltrated microglia stained positively for CD68, a marker for activated microglia. At 1-month post-transplantation, no accumulation of microglia was observed at injected sites of asF5-iPS- and MC23-CLiPS NPC transplanted brains, presumably because they have dispersed and returned to a quiescent state following clearance of the xenografts. Human TH+ neurons survived up to 9 months in some animals transplanted with EC23-CLiPS NPCs as confirmed by human nuclear antigen (HuNu) and human NCAM staining (FIG. 8a-f). The rotation asymmetry test revealed that lesioned animals will exhibit contraversive rotations due to hypersensitivity of post-synaptic D2 dopamine receptors on the lesioned striatum as a result of dopamine depletion, when challenged with the dopamine agonist apomorphine. Efficacy of administered interventions will be manifested as amelioration of this rotation asymmetry. Only animals transplanted with EC23-CLiPS NPCs showed improvement in rotational behaviour for both species in contrast to asF-iPS NPC or sham transplanted animals (FIG. 8h). In these mice, the reduction in rotations reached significance (p<0.05) beginning at Week 20 post-transplantation, decreasing to 18.2±24.7% and 11.1±20.8% at Week 20 and Week 22, respectively. The models showed latency in recovery, with initial observed worsening following transplantation. This is likely due the inflammatory reaction resulting from stereotaxic injection and time required for NPCs to mature, integrate with and innervate host tissues. Functional improvement of parkinsonian motor symptoms in EC23-CLiPS NPC transplanted animals suggests functional recovery of dopaminergic functions in the grafted striatum. To further investigate this, we performed PET imaging with the dopamine transporter (DAT) ligand [18F]FE-PE2I (Bang, J. I., et al., Nucl Med Biol, 2016. 43(2): p. 158-64; Sasaki, T., et al., J Nucl Med, 2012. 53(7): p. 1065-73) in transplanted mice. DAT is a presynaptic transmembrane protein primarily responsible for the reuptake of dopamine released at synapses and molecular imaging of DAT is an established tool for studying dopaminergic functions. PET imaging at 6-months post-transplantation showed recovery of DAT activity in grafted lesioned hemispheres to about 71.4±10.3% (n=3) the activity of non-lesioned hemispheres in EC23-iPS NPC transplanted mice (FIG. 8i). In contrast, the recovery was only 16.4±4.0% in asF-iPS-NPC transplanted mice. These results clearly indicate a significant restoration of dopamine reuptake function in EC23-iPS NPC grafted mice.

### Example 13: Transplantation of CLiPS-Derived Dopaminergic Neurons in Fully Immunocompetent Rodent Rat Models of Parkinson's Disease

Our transplantation results indicate that EC23-CLiPS-derived NPCs are tolerated when transplanted into the striatum of C56BL/6 mice. To rule out possible species-specific bias of this phenomenon, the transplantation study was replicated in a different species, the Wistar rat. Parkinsonism was induced in these rats by the injection of 6-OHDA into the MFB to lesion the nigrostriatal pathway. In this context, it is noted that all animal experiments were conducted according to protocols approved by the Institutional Animal Care and Use Committee (IACUC) of the National Neuroscience Institute (NNI), Singapore. Additional approval for rat experiments was provided by the IACUC of the National Technological University (NTU), Singapore. MFB lesions are known to cause a more complete depletion of the dopamine system compared to striatal lesions and are therefore presumed to be less likely to lead to spontaneous recovery (Torres, E. M. and S. B. Dunnett, Animal Models of Movement Disorders: Volume I, E. L. Lane and S. B. Dunnett, Editors. 2012, Humana Press: Totowa, NJ. p. 267-279). The rats were fully immunocompetent and no immunosuppression was administered prior to or following transplantation. For the analysis, female Wistar rats of ˜8 weeks old were purchased from InVivos Pte Ltd. Unilateral lesion was induced by stereotaxic injection of 20 μg of 6-OHDA in 4 μl into the left medial forebrain bundle (MFB) at the following coordinates: AP −4.4 mm; ML −1.2 mm; and DV −8.6 mm from dura. To determine suitable models for transplantation, apomorphine-induced rotations were scored as described in Example 12. Rats displaying >6 rotations/min were transplanted with 3 μl of about 1.25×10⁵ cells/μl Day25 dopaminergic progenitors into the left striatum at the following coordinates with reference to Bregma: AP +0.8 mm; ML −2.5 mm; and DV −5 mm from dura. To assess whether transplanted NPCs can integrate and mediate functional benefits in lesioned animals, rotation asymmetry tests were performed at 1-monthly intervals as described in Example 12. Rats were sacrificed at 6 months by terminal anesthesia and brains were harvested for immunohistological analyses after transcardial perfusion with 4% PFA as described in Example 12. A few animals that failed the lesioning criteria were also similarly transplanted and sacrificed at 1 and 3 months post-transplantation to assess cell survival and engraftment.

The results show unilateral depletion of dopaminergic neurons in the substantia nigra as a result of retrograde transport of 6-OHDA via the MFB was confirmed in the model by DAB staining for TH in midbrain sections (FIG. 11d). Animals displaying at least 5 turns/minute under apomorphine challenge were transplanted with asF-iPS-, EC23-CLiPS- and MC23-CLiPS-derived NPCs. Histological analyses 3 months post-transplantation showed the presence of hCyto+/HuNu+ and hNCAM+/TH+ cells only in the EC23-CLiPS group. In addition, TH+ neurons in the graft showed expression of Synapsin1, suggesting integration with host neurons (FIG. 11b). Further, only animals transplanted with EC23-CLiPS NPCs showed improvement in rotational behaviour for both species in contrast to asF-iPS NPC or sham transplanted animals (FIG. 11e). Also the rat models showed latency in recovery, with initial observed worsening following transplantation. This is likely due the inflammatory reaction resulting from stereotaxic injection and time required for NPCs to mature, integrate with and innervate host tissues. The results also indicate a significant restoration of dopamine reuptake function in CLiPS-derived NPC grafted rats.

### Example 14: Methods of Differentiating and Characterizing RPE Cells

RPE differentiation from CLiPs was achieved using a rapid and directed differentiation protocol. Differentiated RPE cells were purified and plated on transwells for further characterization, such as immunostaining and gene expression assays using quantitative reverse transcriptase polymerase chain reaction (q-RT-PCR). Functionality of the cells was analysed using transepithelial electric resistance (TEER) and phagocytosis of photoreceptor outer segments (POS).

### Cord Lining Induced Pluripotent Stem Cell (CLiPs) Culture

CLiPs were cultured on Matrigel (Corning) coated tissue culture plate (Corning Costar) in mTESR1 (Stem Cell Technologies) medium.

Differentiation into Retinal Pigmented Epithelium

The inventors used the directed differentiation protocol by Foltz and Clegg (2017) with applying different modifications. CLiPs and human ES cells were grown on Matrigel coated tissue culture plate in mTeSR1 medium. When cells attained 90-95% confluence they were exposed to various differentiation media, containing base medium (DMEM/F12 with 1× B27 and 1× N2 supplements and non-essential amino acids) supplemented with various growth factors.

Differentiation medium 1: from days 0 to 2, μM LDN-193189, 10 ng/ml Dkk1, 10 ng/ml IGF1 and 10 mM nicotinamide. Differentiation medium 2: from days 2 to 4, 0. μM LDN-193189, 10 ng/ml Dkk1, 10 ng/ml IGF1, 10 mM nicotinamide and 5 ng/ml b-FGF. Differentiation medium 3: from days 4 to 6, 10 ng/ml Dkk1 and 10 ng/ml IGF1 and 100 ng/ml Activin A. Differentiation medium 4: From days 6 to 8, 100 ng/ml Activin A and 1 μM SU5402. Differentiation medium 5A: From days 8-11, the basal medium contained 100 ng/mL Activin A, 1 μM SU5402, and 1.5 m CHIR99021. Differentiation medium 5B: from days 11-16, 100 ng/mL Activin A, 1 μM SU5402, and 3 μM CHIR99021. At day 16, base medium was replaced by RPE maintenance medium: 50% DMEM/F12, 50% minimum essential medium Eagle, Alpha Modification, 10 mM nicotinamide, penicillin/streptomycin, sodium pyruvate, MEM non-essential amino acids, GlutaMAX (all 1:100), N1 supplement (1:200), 0.25 mg/ml taurine, 0.02 g/ml hydrocortisone, and 0.013 ng/ml 3,3′,5-Triiodo-L-thyronine supplemented with 2% heat-inactivated fetal bovine serum (FBS). The medium was changed every 2-3 days. In the modified protocol Su5402 in differentiation mediums 4, 5A and 5B were replaced with 1 μM PD173074.

### Characterization of CLiPs-Derived RPE

Transcriptional assay—Quantitative Reverse-Transcriptase Polymerase Chain Reaction
Immunocytochemistry to evaluate RPE-specific proteins
Transepithelial Resistance (TEER)
Photoreceptor Outer Segment (POS) Phagocytosis Assay
   Quantitative Reverse-Transcriptase Polymerase Chain Reaction (qRT-PCR)

RNA samples were collected on days 0, 2, 4, 6, 8, 12, 16, and D30. Total RNA was isolated using the RNeasy Mini Kit (Qiagen). cDNA was synthesized from 1 g of RNA using the iScript cDNA Synthesis Kit (Bio-Rad). qRT-PCR was done in technical triplicates (10 l reactions) in a 96-well plate using KAPA SYBR FAST on a Quant Studio 3 Real-Time PCR Systems (Thermo Fischer). Gene specific primers designed to create 75-200 base pair PCR product included OCT4, NANOG and SOX2 for pluripotency markers, OTX2, LHX2, RAX and SIX3 as early eye field markers, PAX6, MITF, VSX2 and SOX10 as early RPE markers, BEST-1, PMEL 17, MERTK, TYROSINASE, TRYP2, and RPE65 as mature RPE markers. The data were normalized to the “housekeeping” gene glyceraldehyde phosphate dehydrogenase (GAPDH).

### Immunocytochemistry

6 weeks after seeding on transwells, cells were washed with PBS and fixed with 4% paraformaldehyde (pH 7.4) for 20 min at room temperature (RT). The fixed cells were then washed with PBS, permeabilized with 0.2% Triton X-100 for 3-5 minutes at RT and blocked with 1% bovine serum albumin (BSA) in PBS for 1 hour at RT. The cells were then probed with primary antibodies in 1% BSA overnight at 4° C. Following 3 washes to remove the primary antibodies, cells were incubated with the appropriate Alexa Fluor-conjugated secondary antibody (1:1000; Life Technologies), DAPI to stain nuclei and Alexa conjugated phalloidin to stain actin, for 45 minutes at RT. Cells were washed with PBS and mounted using Fluorsave (Calbiochem). Cells were imaged using an LSM 700 confocal microscope (Carl Zeiss, Jena, Germany) using 40× or 63× oil immersion objectives.

Following antibodies were used: rabbit zona-occludens 1 (ZO-1, 1:200, Invitrogen), rabbit Occludin (1:125, Invitrogen), mouse Retinal Pigment Epithelium-Specific 65 KDa Protein (RPE65, 1:125, Abcam), mouse cellular retinaldehyde-binding protein (CRALBP, 1:1000, Abcam), mouse bestrophin (BEST-1, 1:125, Abcam), mouse Na+/ATPase (1:250, Invitrogen), mouse Ezrin (1:200, Abcam) and mouse claudin 19 (1:125).

### Transepithelial Resistance (TEER)

Development of epithelial barrier properties and tight junction formation between the RPE cells reflecting integrity and polarity of RPE monolayer was determined by measuring TEER. For this, cells were cultured on permeable 0.4-μm 24-well Transwell inserts (Corning) coated with Synthemax-II (Corning). TEER measurements were taken every week, using the Epithelial Volt Ohm meter—EVOM2 (World Precision Instruments) following the manufacturer's instructions. Briefly, electrodes were sterilized with 70% ethanol, air dried and equilibrated in RPE medium and placed in the transwell filter with the longer electrode positioned in the lower chamber touching the bottom of the dish and the shorter electrode in the upper chamber. Net TEER (Ω·cm2) was calculated by subtracting the resistance values of experimental transwells from those of controls, transwells in which no cells were plated, and multiplying net values by the area of the filter membrane.

### Photoreceptor Outer Segment (POS) Phagocytosis Assay

POS was isolated from porcine eyes collected from a local abattoir. They were halved with blade and the retinas were removed using forceps in a dark room under red light. Retinas were placed in the homogenisation medium, thoroughly mixed and filtered. The retina suspension was layered on top of a sucrose gradient (25-60%) and centrifuged at 112,398×g for 1 hour in an ultracentrifuge (Optima ultracentrifuge, Beckman). The pink POS layer was collected and POS were labelled with fluorescein isothiocyanate (FITC, Invitrogen) in 0.1M Sodium bicarbonate buffer (pH 9.5) for 1 hour at RT. The labelled POS was washed and stored as aliquots in −80° C. till use. For phagocytosis assay, the RPE cells growing on transwells were challenged with FITC labelled POS for 2 hours at 37° C. in 5% CO2 incubator or 4° C. for control. Cells challenged with unlabelled POS were used as controls. The wells were then washed thrice with PBS to remove unbound POS and dissociated to single cells using TrypLE (Gibco). FITC fluorescence was measured using BD LSR II Flow Cytometer to determine POS phagocytosis.

### Flow Cytometry

4-6 week old cells grown on 24 well plates were stained with Pmel17 antibody (Dako, M0634) using IntraPrep Permeabilization Reagent (Beckman Coulter, IM2389). Briefly, cells were dissociated with TrypLE and fixed and permeabilised according to instruction provided in the kit. About 50,000 cells were transferred to 15 ml centrifuge tubes and incubated with 2-4 μl Pmel or RPE65 antibody overnight on a roller shaker in the cold room, followed by washing with PBS the cells were incubated with Alexa-labelled 488/647 secondary antibodies and analysed in FACS LSR5 II machine (BD).

### RPE Purification Methods

(i). Manual purification: Non-RPE cells in differentiation cultures were removed manually by scraping with a 10 μl tip attached to the pipette by observing the culture plate under a dissection microscope. The plate was washed with PBS, 3 times to remove all the non-RPE cells. The remaining cells, highly enriched in RPEs, were dissociated by adding fresh TrypLE and incubation for 5-10 minutes.

(ii). TrypLE purification: RPE cells have stronger attachment to the tissue culture plates compared to non-RPE cells. Due to the weaker attachment to the growth surface, non-RPE cells can be easily removed by treatment with gentle dissociation agents like TrypLE. After aspirating culture medium, phosphate buffered saline was added to the wells and loosely attached non-RPE clumps were removed by vigorous pipetting with a 1 ml pipette. After removal of the detached clusters, plates were incubated with TrypLE for 5-10 minutes and non-RPE cells were detached by gentle tapping and pipetting to release the non-RPE cells. The detached non-RPE cells were collected, dissociated and used as the “loose” fraction. The plate was thoroughly washed with PBS, 3 times to remove all the non-RPE cells and the tightly attached RPE cells were dissociated by incubating with fresh TrypLE for 5-10 minutes. Both tightly attached RPE and loosely attached non-RPE cells were plated for experiments.

(iii). TrypLE+Manual purification: Although the TrypLE purification described above removed most of the big non-RPE clumps, some small clumps were still present which were removed by manual purification as described in method 1.

(iv) TrypLE+scatter sorting: After aspirating the culture medium, loosely attached RPE cells were removed as described in method 3. The remaining cells were dissociated by further TrypLE treatment for 5-10 minutes and processed as described in scatter sorting. During sorting, the weakly attached non-RPE cells removed by TrypLE treatment was used to set the gate for scatter low cells. The scatter high population present only in the strongly attached fraction helps to set the gate to sort RPE cells.

(v) Scatter sorting: All cells on the differentiation plate were dissociated to single cells by TrypLE treatment and pelleted. The cell pellet was resuspended in FACS buffer was passed through a 70 μm filter to get single cells and separated into scatter high and low fractions using in BD FACS Aria II cell sorter.

### Analysis of Mitochondrial and Glycolytic Functions

Analysis of mitochondrial and glycolytic functions was performed for CLiPs-RPE, skin-iPSC-RPE and H9-RPE cells using XFe96 Extracellular Flux Analyzer (Seahorse Bioscience) using the assay conditions described by the manufacturer. Cells were plated at seeding density of 6×10⁴ in Synthemax-II coated 96 well plate and grown for 48 hours. Oxygen consumption rate (OCR) was detected under basal conditions followed by the sequential addition of Oligomycin (2 μm), FCCP (1.5 μm), as well as rotenone (0.5 μM) and Antimycin A (0.5 μM) using Cell Mito Stress Test. This allowed for measurement of the following parameters: basal respiration, OCR, ATP production, maximal respiration, spare respiratory capacity, and non-mitochondrial respiration. Extra cellular acidification rate (ECAR) was measured under basal conditions followed by the sequential addition of 10 mM glucose, 5 μM oligomycin, and 100 mM 2-DG, using Glycolytic Stress Test. This measured the parameters of glycolytic capacity and glycolytic reserve. Assay results were normalised to cell number.

### Image Analysis to Assess Pigmentation

Black and white 16-bit images of differentiation plates were taken at regular intervals from day 17 using Chemidoc touch system (Biorad). Based on the background intensity threshold, the black area of RPE cells and their brightness of was measured using Matlab software. The brightness of pigmentation obtained from analysis was subtracted from the maximum possible intensity of brightness for a 16-bit image (65535) to get the darkness of pigmentation.

### Example 15: CLiPS Differentiate into RPE Cells

The inventors used an RPE differentiation method to generate RPE from CLiPS, which can be of either mesenchymal (CLMC) or ectodermal (CLEC) origin (Table 4).

TABLE 4
Different stem cells used in this study

| | | Age of | Gender of | Ethnic background |
|---|---|---|---|---|
| Cell line | Cell type | donor | donor | of donor |
| AsF5 | Skin iPS | 78 | Male | Asian |
| AG04148 | Skin iPS | 56 | Male | Caucasian |
| HDFA | Skin iPS | 31 | Female | Caucasian |
| CLEC23 | CLEC | 0 | Male | Asian |
| CLMC23 | CLMC | 0 | Male | Asian |
| CLMC30 | CLMC | 0 | Male | Asian |
| CLMC44 | CLMC | 0 | Female | Asian |
| H9 cells | Human ES | 0 | Female | |

The inventors used ES cell and skin iPS cells as controls in RPE differentiation (Table 4). Using this method, CLiPS robustly differentiated into RPEs (FIG. 13).

### Example 16: CLiPS have Consistently High RPE Differentiation Efficiency than Skin iPS Cells

To compare the RPE differentiation efficiency of different types of stem cells (Table 4) the inventors developed a visual grading system (FIG. 14a) by estimating percentage area occupied by pigmented cells on each well of the differentiation plate. RPE differentiation efficiency is graded as 0, 1, 2 or 3 for no pigmentation, <30%, 30-60% or >60% pigmentation, respectively. Stacked column graphs were plotted for each differentiation plate with different colours indicating different grade of pigmentation (FIG. 14b). In biological replicates of differentiation, ES cells and iPS cells derived from cord-lining mesenchymal cells (CLMCs) showed consistently high RPE differentiation efficiency compared to skin iPS cells. High RPE differentiation of CLiPS was further confirmed by estimating the percentage of cells expressing RPE-specific protein Pmel17 by flow cytometry (FIG. 14c).

### Example 17: CLiPS-Derived RPEs have More Pigmentation than ES-Derived RPE

To compare the pigmentation intensity of RPEs generated from various cell lines and kinetics of pigmentation acquisition, images of differentiation plates taken at regular intervals from day 17 of differentiation using ChemiDoc Touch gel imaging system (Bio-Rad laboratories). CLiPS had darker pigmentation than ES cells in images of differentiation plates (FIG. 15a) and phase contrast images (FIG. 15b) taken at day 30. Analysis of pigmentation intensity at different days of differentiation showed CLiPS-RPE had higher pigmentation than ES-derived RPE throughout differentiation (FIG. 15c). In agreement with this, differentiation cultures of CLMC23 showed higher expression of pigmentation associated genes MITF, PMEL17, TYROSINASE and TRYP2 (FIG. 15d).

### Example 18: CLiPS-Derived RPEs Express RPE Specific Genes

The inventors checked expression of RPE specific genes in CLiPS-derived RPE by quantitative PCR at day 18 and 35 of differentiation. Robust expression of RPE specific genes RPE65 and MERTK (FIG. 16) was observed, the levels were higher in CLiPS-derived RPE at day 35 suggesting they could me more mature.

### Example 19: CLiPS-Derived RPEs have Functional Tight Junctions and are Able to Phagocytose

The functionality of CLiPS-RPEs were determined by measuring Trans-epithelial electrical resistance (TEER) and phagocytosis of FITC labelled photoreceptor outer segments. TEER (FIG. 17a) and phagocytosis (FIG. 17b) of CLiPS-RPE were similar to that of ES-derived RPE.

### Example 20: CLiPS-RPEs Express Protein Similar to that of ES-Derived RPEs

Polarization of RPEs are crucial for apico-basal specific functions, proteins show localised expression in the polarised RPE monolayer. To test if CLiPS show expression of proteins similar to that of previously reported RPEs, the inventors immunostained RPEs for different proteins. ZO-1 was expressed at cell-cell junctions, Mertk at the apical side and RPE-65 in the cytoplasm (FIG. 18) similar to native, ES and iPS-derived RPEs.

### Example 21: Modification of RPE Differentiation Protocol

The inventors developed a slightly-modified RPE differentiation protocol based on the method developed by Foltz and Clegg 2017 (J Vis Exp. 2017; (128): 56274). (A) Usage of CHIR at 3 μM (FIG. 19a) as described in the original Clegg protocol resulted in excessive cell death around day 11-12 of differentiation, resulting in a dramatic decrease in RPE yield. To prevent this the inventors modified the protocol by gradually increasing CHIR in the medium in the following way: starting with 1. μM for 3 days (day 8-11 of differentiation) followed by 3 μM for 5 days (day 12-17). (B) New FGF inhibitors were also identified for RPE differentiation: The inventors replaced SU5402 with PD173074 (FIG. 19b) to achieve similar degree of RPE differentiation and pigmentation (FIG. 19c). PD173074 was used at much lower concentration than SU5402 (1 μM instead of 1 μM), which could reduce undesirable changes in gene expression induced by high concentration of the chemical (Waldmann T et al., 2014, Chem Res Toxicol, 2014 Mar. 17; 27(3):408-20). RPEs obtained from differentiation using different FGF inhibitors, SU5402 or PD173074 had comparable TEER and phagocytosis (FIG. 19d, e).

### Example 22: Method Development for RPE Purification

RPE purification was done with 30-35 day old differentiation cultures, by when the RPEs have gained pigmentation. Methods to purify RPE from mixed differentiation cultures include manual removal of non-RPE cells at day 14 of differentiation based on the morphological differences between RPE and non-RPE cells (Foltz and Clegg, 2017), selective removal of non-RPE cells which are weakly attached to the culture dish by short treatment with a weak dissociation agent such as TrypLE or Accutase (Nazari et al., 2015, Yuko Iwasaki et al., 2016) and scatter sorting based on the higher scatter of light by melanosomes in the RPE cells (Shih et al., 2017). The inventors performed RPE purification with 30-35 day old differentiation cultures unlike 14 day old cultures descried previously (Foltz and Clegg, 2017). By 30-35 days, RPE cells in differentiation cultures acquire brown pigmentation which helps to distinguish RPE cells from non-RPE cells in manual purification. The inventors compared different methods (FIGS. 20a and b) to identify which method would give functional RPE of highest purity and yield. These included (i) Manual purification: identification of non-RPE cells based on their morphology and lack of pigmentation and manual removal of them by scraping by observing under a dissection microscope, (ii) TrypLE purification: removal of majority of weakly attached non-RPE clusters by partial TrypLE treatment, (iii) TrypLE+ Manual: elimination of majority of weakly attached non-RPE clusters by partial TrypLE treatment followed by manual removal of few non-RPE clusters that escaped TrypLE treatment by observing under a dissection microscope (iv) TrypLE+scatter sorting: removal of weakly attached non-RPE clusters by partial TrypLE treatment followed by scatter sorting, (v) Scatter sorting: separation of all cells from mixed differentiation culture based on their relative light scatter (FIG. 20c), as scatter-high (pigmented RPE cells) and scatter-low (non-pigmented non-RPE cells) populations. To make selection of gates more precise, the inventors introduced a control for low scatter using the weakly attached non-RPE cells in the differentiation culture (collected from partial TrypLE treatment). RPE cells was sorted and selected based on scatter high gate (FIG. 20d). This two-step purification involving the removal of non-RPE cells by TrypLE first before flow cytometry, helped to reduce sorting time. Percentage yield of RPE cells was calculated by number of RPE cells obtained after purification/total number of cells in mixed population of RPE cells prior to purification×100%.

Since preferential dissociations are expected to remove non-RPE cells, all cells obtained after purification were considered as RPE cells. Percentage yield of RPEs after purification was calculated from total number of cells present in differentiation cultures and the number of cells obtained after purification. For purification the inventors used a differentiation culture from HDFA iPS cells that contained 51% Pmel17 positive cells by flow cytometry, suggesting it contained 51% RPE cells. Hence the maximum achievable RPE yield with this culture was 51%. TrypLE purification yielded 50% RPE cells, very close to the maximum expected yield of 51%. Manual and TrypLE+manual gave slightly lower yields, 47.7 and 43.4% respectively. Methods involving scatter sorting gave low yield (21-22%) (FIG. 20e). All methods of purification achieved more than 95% purity by Pmel17 flow cytometry (FIG. 20f). By 10 weeks TEER values of RPEs purified by different methods were similar (FIG. 20g). In phagocytosis assay, all purification methods yielded RPE cells with more than 90% phagocytosis (FIG. 20h).

In conclusion, RPE cells purified by all methods had comparable purity, TEER and POS Phagocytosis. In terms of yield, Manual, TrypLE and TrypLE+scatter gave the highest RPE yield. TrypLE and TrypLE+manual were the easiest to perform as partial TrypLE treatment removed majority of the non-RPE cells (FIG. 20i). TrypLE purification would be an efficient way to generate RPE cells for most general research purposes because of its ease of purification, high yield, purity and functionality. TrypLE+manual method, which involves an additional manual purification step to remove any non-RPE cells that might have escaped TrypLE treatment, would be ideal for generating RPE cells for transplantation. After purification, the inventors compared the expression of BEST1, RPE65, MERTK, MITF, PMEL17, RLBP1 and TRYP2 in purified CLMC23 and H9 (FIG. 20j). Majority of these genes showed an increased expression in CLMC23 compared to the ES-derived H9 RPE (FIG. 20k).

### Example 23: CLiPS-Derived RPE have High Glycolytic and Mitochondrial Respiration

Measurements of glycolysis and mitochondrial respiration provide indication of bioenergetics and cell health. Bioenergetics of RPE cells derived from different stem cells was measured using XFe96 Extracellular Flux Analyzer (Seahorse Bioscience) using the Cell Mito Stress Test assay conditions. Glycolytic function was quantified by extracellular acidification rate (ECAR) and oxidative phosphorylation (oxPhos) by oxygen consumption rate (OCR). FIGS. 21a and b demonstrate that among the differetiated RPE, CLiPs-RPE cells have a bioenergetic profile closest to the primary RPE (AHRPE) making them physiologically closer to native RPE. Moreover, the CLiPs-RPE also show higher glycolysis and oxidative phosphorylation compared to both skin-iPSC-RPE (ASF5-RPE) and hESC-RPE (H9-RPE) (FIGS. 21a and b). These results suggest CLiPs-RPE have higher bioenergetic profile compared to hESC-cell derived RPE. Healthy RPEs exhibit higher glycolysis and mitochondrial function compared to RPEs from AMD patients (Ferrington et al, 2017). Higher OCR and ECAR of CLiPS-RPE suggest they could be superior to hESC-derived RPE for clinical use. This is evidenced by increased resistance to oxidative stress demonstrated by CLiPs-RPE on exposure to oxidized low-density lipoprotein (FIG. 21c) and hydrogen peroxide (FIG. 21g) compared to skin-iPSC-RPE (FIGS. 21d and h) and hESC-RPE (FIGS. 21e and i) which are susceptible to oxidative stress. This indicates that CLiPs-RPE may survive better after transplantation. CLiPs-RPE cells' response to oxidative stress is similar to that seen in native RPE AHRPE) (FIGS. 21f and j) making them functionally closer to primary RPE compared to other differentiated RPE.

### Example 24: CLiPS RPE have Potential Immune-Privilege Properties in Humanised Mouse Models

Bioluminescent RPE lines were established using a luciferase (Luc) gene-encoding vector tagged with GFP, delivered via lentiviral infection. Stable expression of Luc in these lines was confirmed by analyzing the bioluminescent intensity. To test the immunogenicity of the different RPE lines, the matrigel plug assay was adopted from previous publication (PMID: 15780993). The RPE-matrigel plugs were transplanted subcutaneously in humanized mice. The bioluminescence of RPE-matrigel plugs were monitored at regular intervals over a time course of 2 months using the bioluminescent imaging system. The total radiance (bioluminescence) for all RPE lines in humanized mice showed a slight, but insignificant decrease in signal over time (FIG. 22a). This indicated that the RPE cells did not overtly proliferate and remained in their expected quiescent state typical of matured RPE. To discern if the slight decrease observed over time was due to the clearance by the immune system, RPE-matrigel plugs were transplanted into NOD-SCID IL2Rγ−/− (NSG) immunodeficient mice. The same decrease in total radiance was observed, ruling out RPE cell clearance by immune system (FIG. 22b). This decrease could otherwise be attributed to the lack of nutrient supply to the area resulting in gradual cell death over experimental period. To confirm the survival and determine the state of RPE cells in the matrigel-RPE plugs, the graft was extracted at end-point and immunofluorescence analysis was conducted using a mature RPE marker (RPE65) and a proliferation marker (Ki67) (FIG. 22c). In all RPE lines tested, the expression of RPE65 was observed and Ki67 was absent, confirming the mature and quiescent state.

### Example 25: Monitoring of Pro-Inflammatory Cytokines as a Surrogate for Cellular Immune Response

After confirming the survival of mature RPE cell graft in all groups, the serum samples collected at the end-point of humanized mice were tested for the presence of key pro-inflammatory cytokines (IFN-γ and IL-18), which are the central effectors of cell-mediated immunity (PMID: 29856726). All RPE lines tested showed cytokine levels in the range of picograms, which was below the threshold that induces systemic immune system activation (FIGS. 23a and b). Unexpectedly, for both cytokines, CLEC23-RPE consistently showed the lowest levels compared to the other lines. The immune response was then studied at a localized level, that is, in the RPE-matrigel plug, to detect a localized immune reaction. Immunofluorescence analysis was conducted to observe immune cell infiltration using human CD45 (hCD45) marker. OTX2, a RPE-specific transcription factor, was used to distinguish the RPE cells (FIG. 23c). Congruent with the low levels of IFN-γ and IL-18 cytokines, immune cell infiltration was indeed absent in CLEC23-RPE. A qualitative grading (Grade 0 to 3) based on hCD45 positive cells was carried out to determine the severity of immune infiltration from all groups (at least n=3) and plotted (FIGS. 23d and e). CLEC23-RPE had the lowest immune cell infiltration. These data allude to CLEC23-RPE potentially having immune-privilege properties.

### Example 26: CLEC23-RPE May Modulate T Cell Activation to Confer Hypo-Immunogenicity

Transplant rejections are primarily caused by cell-mediated immune responses. As observed from the previous figures, CLEC23-RPE had the lowest immune cell infiltration. Therefore, we hypothesized that CLEC23-RPE may affect the activation of elements involved cell-mediated immunity, the T cells. IL-23 and IL17A, known cytokines involved in T cell activation, were analyzed in the humanized mice serum (FIGS. 24a and b) (PMID: 26252407). CLEC23-RPE showed the lowest levels of both cytokines compared to the other groups. Since these cytokines affect T cell activation, the relative ratio of T cells (CD3) to B cells (CD19) was calculated using flow cytometry (FIG. 24c). Only CLEC23-RPE group had a lower T cell count relative to B cell, pointing to the notion that T cell activation may be suppressed. An additional analysis on the subsets of T cell, helper T (CD4) and cytotoxic T (CD8) cells was performed and showed that CLEC23-RPE has the lowest cytotoxic T cells relative to helper T cells (FIG. 24d). The activation status of these two subsets were then further categorized into four groups, naïve (quiescent), central memory (CM; pre-activation) and two activated states, effector memory (EM) and effector memory re-expressing CD45RA (TEMRA). There was no obvious different between the activation states of the CD4 helper T cell subset (FIG. 24e). However, in the CD8 cytotoxic T cell subset, CLEC23-RPE clearly showed a higher naïve population (FIG. 24f). Therefore, the inventors postulate that CLEC23-RPE's potential immune-privilege status might arise from the suppression of CD8 cytotoxic T cell activation.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The term “less than” or in turn “more than” or “below” does not include the concrete number. The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. The term “and/or” wherever used herein includes the meaning of “and”, “or” and “all or any other combination of the elements connected by said term”. The term “including” means “including but not limited to”. “Including” and “including but not limited to” are used interchangeably. The term “about” means plus or minus 20%, preferably plus or minus 10%, more preferably plus or minus 5%, most preferably plus or minus 1%.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. When used herein “consisting of” excludes any element, step, or ingredient not specified in the claim element. When used herein, “consisting essentially of” does not exclude materials or steps that do not materially affect the basic and novel characteristics of the claim. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention. The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group. Further embodiments of the invention will become apparent from the following claims.

The present invention is further characterized by the following items:
1. A method of generating an induced pluripotent stem cell, wherein the method comprises expressing exogenous nucleic acids encoding proteins OCT3/4, SOX2, KLF4, LIN28 and L-MYC and p53-shRNA in a stem cell of the amniotic membrane of the umbilical cord under conditions suitable to reprogram the stem cell, thereby generating the induced pluripotent stem cell.
2. The method of item 1, wherein the stem cell of the amniotic membrane of the umbilical cord is a mesenchymal stem cell of the amniotic membrane of the umbilical cord or an epithelial stem cell of the amniotic membrane of the umbilical cord.
3. The method of item 1 or 2, wherein the mesenchymal stem of the amniotic membrane of the umbilical cord is a mesenchymal stem cell population, wherein at least about 90% or more cells of the stem cell population express each of the following markers: CD73, CD90 and CD105.
4. The method of item 3, wherein at least about 90% or more cells of the mesenchymal stem cell population lack expression of the following markers: CD34, CD45 and HLA-DR.
5. The method of any one of items 3 or 4, wherein at least about 91% or more, about 92% or more, about 93% or more, about 94% or more, about 95% or more, about 96% or more, about 97% or more, about 98% or more about 99% or more cells of the mesenchymal stem cell population express each of CD73, CD90 and CD105 and lack expression of each of CD34, CD45 and HLA-DR.
6. The method of any one of items 1 to 5, wherein the exogenous nucleic acids encoding the proteins OCT3/4, SOX2, KLF4, LIN28 and L-MYC and the p53-shRNA are provided by one, two or three vectors, wherein preferably a first vector encodes the protein OCT3/4 and the 53-shRNA, a second vector encodes the proteins SOX2 and KLF4 and a third vector encodes the proteins L-MYC and LIN28.
7. The method of any one of items 1 to 6, wherein the stem cell of the amniotic membrane of the umbilical cord is subjected to transfection to transfer the exogenous nucleic acids into the stem cell.
8. The method of item 7, wherein the stem cell of the amniotic membrane of the umbilical cord is subjected to electroporation to transfer the exogenous nucleic acids into the stem cell.
9. The method of item 8, wherein the mesenchymal stem cell of the amniotic membrane of the umbilical cord is subjected to electroporation with 1 pulse having a duration time of about 15-25 ms and a voltage of about 1550-1650V, preferably to electroporation with 1 pulse having a duration time of about 20 ms and a voltage of about 1600V.
10. The method of item 9, wherein the ratio of the amount of vector (plasmid) DNA for each vector to the number of mesenchymal stem cells of the amniotic membrane of the umbilical cord subjected to electroporation is in the range of about 1.5 μg plasmid DNA to about 1×10⁶ CLMC to of about 2.5 μg DNA to about 1×10⁶ CLMC, wherein the ratio is, for example, about 2.5 μg plasmid DNA: 1×10⁶ cells, about 2.25 μg plasmid DNA: 1×10⁶ cells, about 1.8 μg plasmid DNA: 1×10⁶ cells, about 1.7 μg plasmid DNA: 1×10⁶ cells, about 1.6 μg plasmid DNA: 1×10⁶ cells, about 1.5 μg plasmid DNA: 1×10⁶ cells, or preferably about 1.67:1×10⁶ cells.
11. The method of item 8, wherein the epithelial stem cell of the amniotic membrane of the umbilical cord is subjected to electroporation with 2 pulses having a duration time of about 25-35 ms and a voltage of about 1300-1400V, preferably to electroporation with 2 pulses having a duration time of about 30 ms and a voltage of about 1350V.
12. The method of item 11, wherein the ratio of the amount of vector (plasmid) DNA for each vector to the number of epithelial stem cells of the amniotic membrane of the umbilical cells subjected to electroporation is in the range of about 1.5 μg DNA to about 1×10⁶ cells to about 2.5 μg DNA to about 1×10⁶ cells, wherein the ratio is, for example, about 1.5 μg plasmid DNA: 1×10⁶ cells, about 1.6 μg plasmid DNA: 1×10⁶ cells, about 1.7 μg plasmid DNA: 1×10⁶ cells, about 1.8 μg plasmid DNA: 1×10⁶ cells, about 1.9 μg plasmid DNA: 1×10⁶ cells, about 2.0 μg plasmid DNA: 1×10⁶ cells, about 2.5 μg plasmid DNA: 1×10⁶ cells, preferably about 1.67 μg plasmid DNA: 1×10⁶ cells.
13. The method of any one of items 7 to 12, wherein the transfected stem cell is cultivated in a medium suitable for cell recovery.
14. The method of item 13, wherein the medium suitable for cell recovery is a serum-free medium.
15. The method of item 13, wherein the medium suitable for the recovery of a transfected mesenchymal stem cell of the amniotic membrane of the umbilical cord consists of about 85 to 95% (v/v) defined medium and 5 to 15% (v/v) fetal bovine serum.
16. The medium of item 15, wherein the medium suitable for the recovery of a transfected mesenchymal stem cell of the amniotic membrane of the umbilical cord consists of about 90% (v/v) chemically defined medium and about 10% (v/v) fetal bovine serum.
17. The medium of any of any of items 14 or 15, wherein the medium contains about 85 to 95% (v/v) CMRL 1066 and about 5 to 15% (v/v) FBS.
18. The method of item 13 or 14, wherein the medium suitable for the recovery of a transfected epithelial stem cell of the amniotic membrane of the umbilical cord comprises Mammary Epithelial Basal Medium MCDB 170, EpiLife medium, DMEM (Dulbecco's modified eagle medium), F12 (Ham's F12 Medium) and FBS (Fetal Bovine Serum).
19. The method of item 18, wherein the medium suitable for the recovery of a transfected epithelial stem cell of the amniotic membrane of the umbilical cord comprises Mammary Epithelial Basal Medium MCDB 170 in a final concentration of about 10 to about 30% (v/v), EpiLife medium in a final concentration of about 20 to about 40% (v/v), F12 in a final concentration of about 5 to about 15% (v/v), DMEM in a final concentration of about 30 to about 45% (v/v) and FBS in a final concentration of about 0.1 to 2% (v/v).
20. The method of item 19, wherein the medium suitable for the recovery of a transfected epithelial stem cell of the amniotic membrane of the umbilical cord comprises Mammary Epithelial Basal Medium MCDB 170 in a final concentration of about 15 to about 25% (v/v), EpiLife medium in a final concentration of about 25 to about 35% (v/v), F12 in a final concentration of about 7.5 to about 13% (v/v), DMEM in a final concentration of about 35 to about 40% (v/v) and FBS in a final concentration of about 0.5 to 1.5% (v/v).
21. The method of item 20, wherein the medium suitable for the recovery of a transfected epithelial stem cell of the amniotic membrane of the umbilical cord comprises Mammary Epithelial Basal Medium MCDB 170 in a final concentration of about 20% (v/v), EpiLife medium in a final concentration of about 30% (v/v), F12 in a final concentration of about 12.5 (v/v), DMEM in a final concentration of about 37.5% (v/v) and FBS in a final concentration of about 1.0% (v/v).
22. The method of any of items 18 to 21, wherein the medium suitable for the recovery of a transfected epithelial stem cell of the amniotic membrane of the umbilical cord is obtained by mixing to obtain a final volume of 1000 ml culture medium:
   200 ml Mammary Epithelial Basal Medium MCDB 170
   300 ml EpiLife medium
   250 ml DMEM
   250 ml DMEM/F12
   1% Fetal Bovine Serum.
23. The method of any of items 18 to 22, wherein the medium suitable for the recovery of a transfected epithelial stem cell of the amniotic membrane of the umbilical cord comprises insulin in a final concentration of about 1 to about 7.5 μg/ml.
24. The method of any of items 18 to 24, wherein the medium suitable for the recovery of a transfected epithelial stem cell of the amniotic membrane of the umbilical cord comprises human epidermal growth factor in a final concentration of about 1 to about 15 ng/ml.
25. The method of any of items 18 to 25, wherein the medium suitable for the recovery of a transfected epithelial stem cell of the amniotic membrane of the umbilical cord further comprises at least one of the following supplements: adenine, hydrocortisone, and 3,3′,5-Triiodo-L-thyronine sodium salt (T3).
26. The method of item 25, wherein the medium suitable for the recovery of a transfected epithelial stem cell of the amniotic membrane of the umbilical cord comprises all three of adenine, hydrocortisone, and 3,3′,5-Triiodo-L-thyronine sodium salt (T3).
27. The method of any of items 18 to 26, wherein the medium suitable for the recovery of a transfected epithelial stem cell of the amniotic membrane of the umbilical cord further comprises one of more Transforming Growth Factors (TGF).
28. The method of item 27, wherein the medium comprises Transforming Growth Factor beta (TGF-beta) and/or transforming growth factor alpha.
29. The method of any of items 18 to 28, wherein the medium suitable for the recovery of a transfected epithelial stem cell of the amniotic membrane of the umbilical cord further comprises Cholera Toxin from Vibrio cholerae.
30. The method of any one of items 14 to 29, wherein the medium suitable for cell recovery contains a compound suppressing inflammatory response and enhancing cell survival.
31. The method of item 30, wherein the compound is a glucocorticoid.
32. The method of item 31, wherein the glucocorticoid is selected from the group consisting of prednisolone, methylprednisolone, dexamethasone, betamethasone, corticosterone and hydrocortisone.
33. The method of item 31 or 32, wherein the hydrocortisone concentration is about 0.5 μM to about 2 μM.
34. The method of any one of items 13 to 33, wherein the cultivation is carried out in a coated cell culture vessel, wherein the cell culture vessel is preferably coated with a serum-derived substrate or a serum-free substrate.
35. The method of any one of items 14 to 34, wherein the medium suitable for cell recovery is replaced with a mixture of two different cell culture media about 1, 2 or 3 days after transfection, preferably about 2 days after transfection, thereby yielding an induced pluripotent stem cell colony.
36. The method of item 35, wherein the two different cell culture media are the medium suitable for cell recovery and a second cell culture medium.
37. The method of item 35 or 36, wherein the two different cell culture media are mixed in a ratio of about 1:1 (v/v) prepared by contacting 1 volume medium suitable for cell recovery to 1 volume second cell culture medium.
38. The method of item 36 or 37, wherein the second cell culture medium is a maintenance medium for cultivation of induced pluripotent stem cells, wherein the medium is preferably selected from the group consisting of mTeSR1, StemMACS™ iPS-Brew XF, TeSR™ E8, mTeSRTMPlus, TeSRTM2, mTeSRTM1, Corning® NutriStem® hPSC XF Medium, Essential 8 Medium, StemFlex, StemFit Basic02 and PluriSTEM.
39. The method of any one of items 35 to 38, wherein the mixture of cell culture media is replaced with the same mixture of cell culture media within about 3, 4 or 5 days after transfection, preferably about 4 days after transfection.
40. The method of any one of items 35 to 39, wherein the mixture of cell culture media is replaced with the second cell culture medium within about 5, 6 or 7 days after transfection, preferably about 6 days after transfection.
41. The method of item 40, wherein the second cell culture medium is changed daily or every second day, third day, preferably every second day.
42. The method of item 40 or 41, wherein an induced pluripotent stem cell colony is selected when reaching a size of about 0.5 mm to about 1.5 mm in diameter, and the selected induced pluripotent stem colony is transferred to a coated cell culture vessel for cultivation and proliferation.
43. The method of item 42, wherein the induced pluripotent stem cell colony is selected under bright field microscopy.
44. The method of item 42 or 43, wherein the cell culture medium is changed daily or every second day, preferably every day.
45. The method of any one of items 43 to 44, wherein the induced pluripotent stem cell colony is detached from the coated cell culture device when reaching a confluence of about 50%.
46. The method of item 45, wherein the induced pluripotent stem cell colony is detached with a reagent selected from the group consisting of dissociation reagent, a dispase or an EDTA solution.
47. The method of item 45 or 46, wherein a cell population formed from the induced pluripotent stem cell colony is passaged when reaching about 60-90% confluence, preferably when reaching 70-80% confluence.
48. The method of item 47, wherein the cell population formed from the induced pluripotent stem cell colony is passaged in a ratio of about 1:3 (v/v), wherein the passaging in a ratio of about 1:3 (v/v) is performed by dividing about 1 volume dissociated induced pluripotent stem cells into about 2 volumes of dissociated induced pluripotent stem cells.
49. The method of item 47 or 48, wherein the cell population formed from the induced pluripotent stem cell colony is dissociated with about 0.5 mM EDTA for passaging.
50. The method of item 48 or 49, wherein the passaged cell population formed from the induced pluripotent stem cell colony is cultivated in a medium containing a substance enhancing the survival of the induced pluripotent stem cell.
51. The method of item 50, wherein the substance enhancing the survival of the induced pluripotent stem cell colony is a ROCK inhibitor.
52. An induced pluripotent stem cell population obtainable by the method as defined in any of items 1 to 51.
53. An induced pluripotent stem cell population obtained by the method as defined in any of items 1 to 51.
54. A pharmaceutical composition comprising an induced pluripotent stem cell as defined in item 52 or 53.
55. A method of differentiating an induced pluripotent stem cell as defined in item 52 or 53 into a target cell, wherein the induced pluripotent stem cell is differentiated into the target cell under conditions suitable for differentiation.
56. The method of item 55, wherein the target cell is selected from the group consisting of a dopaminergic neuronal cell, an oligodentrocyte, a hepatocyte, a cardiomyocyte, a hematopoietic progenitor cell, a blood cell, a neuronal cell, a motor neuron, a cartilage cell, a muscle cell, a bone cell, a dental cell, a hair follicle cell, an inner ear hair cell, a skin cell, a melanocyte, an immune cell, an astrocyte, a reproductive cell, a corneal cell, an intestinal cell, a lung cell, a kidney cell, a stomach cell, a mesenteric cell, and a fat cell.
57. The method of item 56, wherein the immune cell is selected from the group consisting of a T-lympocyte, a B-lymphocyte, a microglia, and a natural killer cell.
58. The method of item 56, wherein the induced pluripotent stem cell is cultivated in a medium adapted for proliferation and differentiation of the induced pluripotent stem cell into a dopaminergic neuronal cell.
59. The method of item 56, wherein the induced pluripotent stem cell is cultivated in a medium adapted for proliferation and differentiation of the induced pluripotent stem cell into a hepatocyte.
60. The method of item 56, wherein the induced pluripotent stem cell is cultivated in a medium adapted for proliferation and differentiation of the induced pluripotent stem cell into a cardiomyocyte.
61. The method of item 60, wherein the induced pluripotent stem cell is cultivated in a medium adapted for proliferation and differentiation of the induced pluripotent stem cell into an oligodentrocyte.
62. A pharmaceutical composition comprising a differentiated induced pluripotent stem cell obtained by the method as defined in any of items 56 to 61.
63. The pharmaceutical composition of item 62, wherein the pharmaceutical composition is adapted for parenteral application.
64. A method of treating a congenital or acquired degenerative disorder in a subject, comprising administering to a subject a target cell differentiated from pluripotent stem cell by the method as defined in any of items 56 to 61.
65. The method of item 64, wherein the disorder is a neural disorder.
66. The method of item 65, wherein the disease is neural disorder is selected from the group consisting of Parkinson's disease, Alzheimer's disease, Huntington's disease, Amyotrophic lateral sclerosis, multiple sclerosis and batten disease.
67. The method of item 64, wherein the disorder is a hepatic disorder.
68. An extracellular membranous vesicle produced by an induced pluripotent stem cell population as defined in item 52 or 53 or produced by a cell obtained by differentiation of an induced pluripotent stem cell as defined in item 52 or 53.
69. The extracellular membranous vesicle of item 68, wherein the vesicle is an exosome.
70. The use of an extracellular membranous vesicle as defined in item 68 or 69 as delivery carrier of a therapeutic agent.
71. A cell culture medium comprising Mammary Epithelial Basal Medium MCDB 170, EpiLife medium, DMEM (Dulbecco's modified eagle medium), F12 (Ham's F12 Medium) and FBS (Fetal Bovine Serum).
72. The cell culture medium of item 71, wherein the medium comprises Mammary Epithelial Basal Medium MCDB 170 in a final concentration of about 10 to about 30% (v/v), EpiLife medium in a final concentration of about 20 to about 40% (v/v), F12 in a final concentration of about 5 to about 15% (v/v), DMEM in a final concentration of about 30 to about 45% (v/v) and FBS in a final concentration of about 0.1 to 2% (v/v).
73. The cell culture medium of item 72, wherein the medium comprises Mammary Epithelial Basal Medium MCDB 170 in a final concentration of about 15 to about 25% (v/v), EpiLife medium in a final concentration of about 25 to about 35% (v/v), F12 in a final concentration of about 7.5 to about 13% (v/v), DMEM in a final concentration of about 35 to about 40% (v/v) and FBS in a final concentration of about 0.5 to 1.5% (v/v).
74. The cell culture medium of item 73, wherein the medium comprises Mammary Epithelial Basal Medium MCDB 170 in a final concentration of about 20% (v/v), EpiLife medium in a final concentration of about 30% (v/v), F12 in a final concentration of about 12.5 (v/v), DMEM in a final concentration of about 37.5% (v/v) and FBS in a final concentration of about 1.0% (v/v).
75. The cell culture medium of any of items 71 to 74, wherein the medium is obtained by mixing to obtain a final volume of 1000 ml culture medium:
   200 ml Mammary Epithelial Basal Medium MCDB 170,
   300 ml EpiLife medium,
   250 ml DMEM,
   250 ml DMEM/F12, and
   1% Fetal Bovine Serum.
76. The cell culture medium of any of items 71 to 75, wherein the medium comprises insulin in a final concentration of about 1 to about 7.5 μg/ml.
77. The cell culture medium of any of items 71 to 76, wherein the medium comprises human epidermal growth factor (EGF) in a final concentration of about 1 to about 15 ng/ml.
78. The cell culture medium of any of items 71 to 77, wherein the medium suitable for the recovery of a transfected epithelial stem cell of the amniotic membrane of the umbilical cord comprises at least one of the following supplements: adenine, hydrocortisone, and 3,3′,5-Triiodo-L-thyronine sodium salt (T3).
79. The cell culture medium of item 78, wherein the medium comprises all three of adenine, hydrocortisone, and 3,3′,5-Triiodo-L-thyronine sodium salt (T3).
80. The cell culture medium of item 79, wherein the culture medium comprises adenine in a final concentration of about 0.05 to about 0.1 mM adenine, hydrocortisone in a final concentration of about 0.1 to 0.5 μM hydrocortisone and/or 3,3′,5-Triiodo-L-thyronine sodium salt (T3) in a final concentration of about 0.1 to about 5 ng/ml.
81. The cell culture medium of any of items 71 to 80, wherein the medium comprises one of more Transforming Growth Factors (TGF).
82. The cell culture medium of item 81, wherein the medium comprises Transforming Growth Factor beta 1 (TGF-beta 1) in a final concentration of about 0.1 to about 5 ng/ml and/or transforming growth factor alpha (TGF-alpha) in a final concentration of about 1.0 to about 10 ng/ml.
83. The medium of any of items 71 to 82, wherein the medium comprises Cholera Toxin from Vibrio cholerae in a final concentration of about 1×10⁻¹¹M to about 1×10⁻¹⁰M.

1. A method of differentiating an induced pluripotent stem (iPS) cell into a retinal pigment epithelial (RPE) cell, the method comprising culturing a iPS cell derived from a stem cell of the amniotic membrane of the umbilical cord in a differentiation medium under conditions suitable for the differentiation into a RPE cell, thereby differentiating the iPS cell into the RPE cell.

2. The method of claim 1, wherein the differentiation medium is DMEM (Dulbecco's modified eagle medium)/F12 (Ham's F12 medium) medium comprising N2 supplement, B27 supplement and non-essential amino acid (NEAA).

3. The method of claim 2, wherein the DMEM/F12 medium comprises 1× N2 supplement, 1× B27 supplement, and 1× NEAA.

4. The method of claim 2 or 3, wherein the differentiation medium is obtained by mixing to obtain a final volume of 1000 ml culture medium:
10 mL of 100× N2 supplement;
20 mL of 50× B27 supplement;
10 mL of 100× NEAA;
960 mL of DMEM/F12.

5. The method of any one of claims 1 to 4, wherein the differentiation medium comprises
i) a first differentiation medium additionally comprising at least any one of IGF1, DKK1, nicotinamide or LDN-193189, preferably comprising IGF1, DKK1, nicotinamide and LDN-193189;
ii) a second differentiation medium additionally comprising at least any one of IGF1, DKK1, nicotinamide, LDN-193189 or b-FGF, preferably comprising IGF1, DKK1, nicotinamide, LDN-193189 and b-FGF;
iii) a third differentiation medium additionally comprising at least any one of IGF1, DKK1 or Activin A, preferably comprising IGF1, DKK1 and Activin A;
iv) a fourth differentiation medium additionally comprising Activin A and either SU5402 or PD173074, preferably comprising Activin A and PD173074; and/or
v) a fifth differentiation medium additionally comprising at least any one of Activin A, CHIR99021, or either SU5402 or PD173074, preferably comprising Activin A, CHIR99021 and PD17307_.

6. The method of claim 5, wherein IGF1 of i), ii) and/or iii) is used in a final concentration of at least 5 ng/ml, preferably wherein IGF1 of i), ii) and/or iii) is used in a final concentration of about 10 ng/ml.

7. The method of claim 5 or 6, wherein DKK1 of claim 5 i), ii) and/or iii) is used in a concentration of at least 5 ng/ml, preferably wherein DKK1 of claim 5 i), ii) and/or iii) is used in a concentration of about 10 ng/ml.

8. The method of any one of claims 5 to 7, wherein nicotinamide of claim 5 i) and/or ii) is used in a concentration of at least 5 mM, preferably wherein nicotinamide of claim 5 i) and/or ii) is used in a concentration of about 10 mM.

9. The method of any one of claims 5 to 8, wherein LDN-193189 of claim 5 i) and/or ii) is used in a concentration of at least 0.1 μM, preferably wherein LDN-193189 of claim 5 i) is used in a concentration of about 1 μM and/or wherein LDN-193189 of claim 5 ii) is used in a concentration of about 0.2 μM.

10. The method of any one of claims 5 to 9, wherein b-FGF of claim 5 ii) is used in a concentration of at least 2.5 ng/ml, preferably wherein b-FGF of claim 5 ii) is used in a concentration of about 5 ng/ml.

11. The method of any one of claims 5 to 10, wherein Activin A of claim 5 iii), iv) and/or v) is used in a concentration of at least 50 ng/ml, preferably wherein Activin A of claim 5 iii), iv) and/or v) is used in a concentration of about 100 ng/ml.

12. The method of any one of claims 5 to 11, wherein SU5402 of claim 5 iv) and/or v) is used in a concentration of at least 5 μM, preferably wherein SU5402 of claim 5 iv) and/or v) is used in a concentration of about 10 μM.

13. The method of any one of claims 5 to 11, wherein PD173074 of claim 5 iv) and/or v) is used in a concentration of at least 0.5 μM, preferably wherein PD173074 of claim 5 iv) and/or v) is used in a concentration of about 1 μM.

14. The method of any one of claims 5 to 13, wherein CHIR99021 of claim 5 v) is used in a concentration of at least 1 μM and less than about 3 μM for culturing the cell, preferably culturing the cell for about 3 consecutive culture days.

15. The method of claim 14, wherein CHIR99021 of claim 5 v) is used in a concentration of about 3 μM for subsequently culturing the cell, preferably subsequently culturing the cell for about 5 consecutive culture days.

16. The method of any one of claims 5 to 15, wherein culturing the iPS cell comprises culturing for about 2 days in the first differentiation medium.

17. The method of any one of claims 5 to 16, wherein culturing the iPS cell comprises culturing for about 2 days in the first differentiation medium, subsequently culturing for about 2 days in the second differentiation medium.

18. The method of any one of claims 5 to 17, wherein culturing the iPS cell comprises culturing for about 2 days in the first differentiation medium, subsequently culturing for about 2 days in the second differentiation medium, subsequently culturing for about 2 days in the third differentiation medium.

19. The method of any one of claims 5 to 18, wherein culturing the iPS cell comprises culturing for about 2 days in the first differentiation medium, subsequently culturing for about 2 days in the second differentiation medium, subsequently culturing for about 2 days in the third differentiation medium, subsequently culturing for about 2 days in the fourth differentiation medium.

20. The method of any one of claims 5 to 19, wherein culturing the iPS cell comprises culturing for about 2 days in the first differentiation medium, subsequently culturing for about 2 days in the second differentiation medium, subsequently culturing for about 2 days in the third differentiation medium, subsequently culturing for about 2 days in the fourth differentiation medium, and subsequently culturing for about 8 days in the fifth differentiation medium.

21. The method of any one of claims 5 to 20, wherein LDN-193189 is used for at least 2 culture days.

22. The method of any one of claims 5 to 21, wherein DKK1 is used for at least 2 culture days.

23. The method of any one of claims 5 to 22, wherein SU5402 or PD173074 is used for about 10 culture days.

24. The method of any one of claims 5 to 23, wherein CHIR99021 is used for about 8 culture days.

25. The method of any one of claims 1 to 24, wherein the iPS cell is cultured in the differentiation medium for 11 to 21 days, preferably for about 16 days.

26. The method of any one of claims 1 to 25, wherein the method further comprises culturing the iPS cell in a mTESR1 medium before culturing the iPS cell in the differentiation medium, preferably culturing the iPS cell in a mTESR1 medium for 1 to 4 culture days.

27. The method of any one of claims 1 to 26, wherein the method further comprises culturing the RPE cell in a retinal pigment epithelial maintenance (RPEM) medium.

28. The method of claim 27, wherein the RPEM medium comprises about 50% DMEM/F12 and about 50% minimum essential medium (MEM) comprising 0.5× N1 supplement and 1× NEAA.

29. The method of claim 28, wherein the RPEM medium further comprises at least any one of a heat-inactivated fetal bovine serum (FBS), Glutamax, taurine, hydrocortisone, 3,3′,5-Triiodo-L-thyronine, penicillin/streptomycin, nicotinamide, or sodium pyruvate.

30. The method of claim 28 or 29, wherein the RPEM medium further comprises about 2% heat-inactivated fetal bovine serum (FBS), 1× Glutamax, about 0.25 mg/mL taurine, about 0.02 μg/mL hydrocortisone, about 0.013 ng/mL 3,3′,5-Triiodo-L-thyronine, 1× penicillin/streptomycin, about 10 mM nicotinamide and 1× sodium pyruvate.

31. The method of any one of claims 27 to 30, wherein the RPE cell is cultured in the RPEM medium for 9 to 29 days, preferably for about 19 days.

32. The method of any one of claims 27 to 31, wherein culturing the iPS cell in the differentiation medium and culturing the RPE cell in the RPEM medium comprises 20 to 50 days, preferably 30 to 35 days, most preferably about 35 days.

33. The method of any one of the claims 27 to 32, wherein the method further comprises purifying the RPE cell in the RPEM medium after culturing said cell in said medium.

34. The method of claim 33, wherein purifying comprises:
a. manually identifying the RPE cell according to their pigmentation;
b. passaging the RPE cell;
c. manually identifying the RPE cell according to their pigmentation and passaging the RPE cell;
d. passaging the RPE cell and scatter sorting the RPE cell according to their pigmentation; and/or
e. scatter sorting the RPE cell according to their pigmentation.

35. The method of claim 34, wherein manually identifying the RPE cell according to their pigmentation of claim 34 a) and/or c) comprises selecting by microscopy.

36. The method of claim 35, wherein microscopy is bright field microscopy.

37. The method of any one of claims 34 to 36, wherein passaging the RPE cell of claim 34 b), c), and/or d) comprises treating the RPE cell with Accutase or TrypLE, preferably with TrypLE.

38. The method of any one of the preceding claims, wherein the iPS cell is generated by expressing exogenous nucleic acids encoding proteins OCT3/4, SOX2, KLF4, LIN28 and L-MYC and p53-shRNA in the stem cell of the amniotic membrane of the umbilical cord under conditions suitable to reprogram the stem cell.

39. The method of claim 38, wherein the stem cell of the amniotic membrane of the umbilical cord is subjected to transfection to transfer the exogenous nucleic acids into the stem cell, wherein the transfected stem cell is cultivated in a medium suitable for cell recovery, wherein the medium suitable for cell recovery contains a compound suppressing inflammatory response and enhancing cell survival.

40. The method of any one of the preceding claims, wherein the stem cell of the amniotic membrane of the umbilical cord is a mesenchymal stem cell of the amniotic membrane of the umbilical cord, or an epithelial stem cell of the amniotic membrane of the umbilical cord.

41. The method of claim 40, wherein the mesenchymal stem of the amniotic membrane of the umbilical cord is a mesenchymal stem cell population, wherein at least 90% or more cells of the stem cell population express each of the following markers: CD73, CD90 and CD105.

42. The method of claim 41, wherein at least 90% or more cells of the mesenchymal stem cell population lack expression of the following markers: CD34, CD45 and HLA-DR.

43. The method of any one of claims 41 to 42, wherein at least 91% or more, 92% or more, 93% or more, 94% or more, 95% or more, 96% or more, 97% or more, 98% or more 99% or more cells of the mesenchymal stem cell population express each of CD73, CD90 and CD105 and lack expression of each of CD34, CD45 and HLA-DR.

44. The method of any one of claims 38 to 43, wherein the exogenous nucleic acids encoding the proteins OCT3/4, SOX2, KLF4, LIN28 and L-MYC and the p53-shRNA are provided by one, two or three vectors, wherein preferably a first vector encodes the protein OCT3/4 and the 53-shRNA, a second vector encodes the proteins SOX2 and KLF4 and a third vector encodes the proteins L-MYC and LIN28.

45. A retinal pigment epithelial (RPE) cell culture obtainable by the method as defined in any of claims 1 to 44.

46. A retinal pigment epithelial (RPE) cell culture obtained by the method as defined in any of claims 1 to 44.

47. A retinal pigment epithelium consisting of or comprising a retinal pigment epithelial cell culture obtainable by the method as defined in any of claims 1 to 44.

48. A retinal pigment epithelium consisting of or comprising a retinal pigment epithelial cell culture obtained by the method as defined in any of claims 1 to 44.

49. A pharmaceutical composition comprising a retinal pigment epithelial (RPE) cell culture obtained by the method as defined in any of claims 1 to 44.

50. The pharmaceutical composition of claim 49, wherein the pharmaceutical composition is adapted for parenteral or local application.

51. The RPE cell culture of any one of claims 45 to 46, the epithelium of any one of claims 47 to 48, and/or the pharmaceutical composition of any one of claims 49 to 50, wherein the RPE cell comprised in said culture expresses at least any one of BEST1, PMEL17, MITF, TYROSINASE, TRYP2, ZO-1, RPE65, RLBP1 or MERTK.

52. The RPE cell culture of any one of claims 45 to 46 and/or of claim 51, the epithelium of any one of claims 47 to 48 and/or of claim 51, and/or the pharmaceutical composition of any one of claims 49 to 50 and/or of claim 51, wherein the RPE cell comprised in said culture expresses BEST1 with a fold change of at least 2 relative to a RPE cell differentiated from an embryonic stem cell (ES).

53. The RPE cell culture of any one of claims 45 to 46 and/or any one of claims 51 to 52, the epithelium of any one of claims 47 to 48 and/or any one of claims 51 to 52, and/or the pharmaceutical composition of any one of claims 49 to 50 and/or any one of claims 51 to 52, wherein the RPE cell comprised in said culture expresses PMEL17 with a fold change of at least 0.9 relative to a RPE cell differentiated from an ES.

54. The RPE cell culture of any one of claims 45 to 46 and/or any one of claims 51 to 53, the epithelium of any one of claims 47 to 48 and/or any one of claims 51 to 53, and/or the pharmaceutical composition of any one of claims 49 to 50 and/or any one of claims 51 to 53, wherein the RPE cell comprised in said culture expresses MITF with a fold change of at least 4.5 relative to a RPE cell differentiated from an ES.

55. The RPE cell culture of any one of any one of claims 45 to 46 and/or any one of claims 51 to 54, the epithelium of any one of claims 47 to 48 and/or any one of claims 51 to 54, and/or the pharmaceutical composition of any one of claims 49 to 50 and/or any one of claims 51 to 54, wherein the RPE cell comprised in said culture expresses TRYP2 with a fold change of at least 2.9 relative to a RPE cell differentiated from an ES.

56. The RPE cell culture of any one of claims 45 to 46 and/or any one of claims 51 to 55, the epithelium of any one of claims 47 to 48 and/or any one of claims 51 to 55, and/or the pharmaceutical composition of any one of claims 49 to 50 and/or any one of claims 51 to 55, wherein the RPE cell comprised in said culture expresses RPE65 with a fold change of at least 0.6 relative to a RPE cell differentiated from an ES.

57. The RPE cell culture of any one of claims 45 to 46 and/or any one of claims 51 to 56, the epithelium of any one of claims 47 to 48 and/or any one of claims 51 to 56, and/or the pharmaceutical composition of any one of claims 49 to 50 and/or any one of claims 51 to 56, wherein the RPE cell comprised in said culture expresses RLBP1 with a fold change of at least 17.5 relative to a RPE cell differentiated from an ES.

58. The RPE cell culture of any one of claims 45 to 46 and/or any one of claims 51 to 57, the epithelium of any one of claims 47 to 48 and/or any one of claims 51 to 57, and/or the pharmaceutical composition of any one of claims 49 to 50 and/or any one of claims 51 to 57, wherein the RPE cell comprised in said culture expresses MERTK with a fold change of at least 6 relative to a RPE cell differentiated from an ES.

59. The RPE cell culture of any one of claims 45 to 46 and/or any one of claims 51 to 58, the epithelium of any one of claims 47 to 48 and/or any one of claims 51 to 58, and/or the pharmaceutical composition of any one of claims 49 to 50 and/or any one of claims 51 to 58, wherein the RPE cell comprised in said culture comprises an increased oxygen consumption rate (OCR) and/or extracellular acidification rate (ECAR) relative to a RPE cell differentiated from an ES.

60. A method of treating a retinal degenerative disease in a subject, comprising administering to a subject a retinal pigment epithelial (RPE) cell differentiated from an induced pluripotent stem (iPS) cell by the method as defined in any of claims 1 to 44.

61. The method of claim 60, wherein the retinal degenerative disease is age-related macular degeneration (AMD) or retinal dystrophy.

62. An in vivo method of detecting the survival rate of a retinal pigment epithelial (RPE) cell differentiated from an induced pluripotent stem (iPS) cell by the method as defined in any of claims 1 to 44 in a subject, the method comprising
a) introducing a RPE cell differentiated from an iPS cell by the method as defined in any of claims 1 to 44 into a subject, wherein said RPE cell comprises a bioluminescence label;
b) detecting the bioluminescence signal of said RPE cell over time using an imaging method, thereby collecting imaging data;
c) comparing the imaging data received in step b) to reference imaging data.

63. The method of claim 62, wherein no difference in the bioluminescence signal in the imaging data from said subject as compared to reference imaging data indicates survival of said RPE cell in said subject.

64. An in vitro method of determining the immunogenicity of a retinal pigment epithelial (RPE) cell differentiated from an induced pluripotent stem (iPS) cell by the method as defined in any of claims 1 to 44 in a subject, to whom said differentiated RPE cell has been pre-delivered, the method comprising:
a) detecting a pro-inflammatory cytokine level using an imaging method in a sample obtained from said subject, the sample comprising said differentiated RPE cell, thereby collecting imaging data;
b) comparing the imaging data received in step a) to reference imaging data.

65. The method of claim 64, wherein a decreased cytokine level in the imaging data as compared to reference imaging data indicates a reduced immunogenicity of said RPE cell in said subject.


SEQUENCE LISTING (50 sequences)

SEQ ID NO: 1 - DNA, 741 bp, Homo sapiens
  source 1..741: mol_type = genomic DNA
ttctttttcctacagctcctgggcaacgtgctggttattgtgctgtctcatcattttggcaaagaattcgcccttcaccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatg

SEQ ID NO: 2 - protein, 352 aa, Homo sapiens
MAGHLASDFAFSPPPGGGDGSAGLEPGWVDPRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSN

SEQ ID NO: 3 - DNA, 949 bp, Homo sapiens
  source 1..949: mol_type = genomic DNA
ccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgcccttcaccatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggc

SEQ ID NO: 4 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 5 - DNA, 1007 bp, Homo sapiens
  source 1..1007: mol_type = genomic DNA
ccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgggggatctcaaaattgtcgctcctgtcaaacaaactcttaactttgatttactcaaactggctggggatgtagaaagcaatccaggtccactcatgaacaattcgcccttcaccatggctgtcagtgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacga

SEQ ID NO: 6 - protein, 513 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQGQSRGFVARAGEPCVCWPHFGTHGMMLTPPSSPLELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 7 - DNA, 679 bp, Homo sapiens
  source 1..679: mol_type = genomic DNA
cttcaccatggactacgactcgtaccagcactatttctacgactatgactgcggggaggatttctaccgctccacggcgcccagcgaggacatctggaagaaattcgagctggtgccatcgccccccacgtcgccgccctggggcttgggtcccggcgcaggggacccggcccccgggattggtcccccggagccgtggcccggagggtgcaccggagacgaagcggaatcccggggccactcgaaaggctggggcaggaactacgcctccatcatacgccgtgactgcatgtggagcggcttctcggcccgggaacggctggagagagctgtgagcgaccggctcgctcctggcgcgccccgggggaacccgcccaaggcgtccgccgccccggactgcactcccagcctcgaagccggcaacccggcgcccgccgccccctgtccgctgggcgaacccaagacccaggcctgctccgggtccgagagcccaagcgactcggagaatgaagaaattgatgttgtgacagtagagaagaggcagtctctgggtattcggaagccggtcaccatcacggtgcgagcagaccccctggatccctgcatgaagcatttccacatctccatccatcagcaacagcacaactatgctgcccgttttcctccagaaagctgctcc

SEQ ID NO: 8 - protein, 364 aa, Homo sapiens
MDYDSYQHYFYDYDCGEDFYRSTAPSEDIWKKFELVPSPPTSPPWGLGPGAGDPAPGIGPPEPWPGGCTGDEAESRGHSKGWGRNYASIIRRDCMWSGFSARERLERAVSDRLAPGAPRGNPPKASAAPDCTPSLEAGNPAPAAPCPLGEPKTQACSGSESPSDSENEEIDVVTVEKRQSLGIRKPVTITVRADPLDPCMKHFHISIHQQQHNYAARFPPESCSQEEASERGPQEEVLERDAAGEKEDEEDEEIVSPPPVESEAAQSCHPKPVSSDTEDVTKRKNHNFLERKRRNDLRSRFLALRDQVPTLASCSKAPKVVILSKALEYLQALVGAEKRMATEKRQLRCRQQQLQKRIAYLTGY

SEQ ID NO: 9 - DNA, 901 bp, Homo sapiens
  source 1..901: mol_type = genomic DNA
ccccctggatccctgcatgaagcatttccacatctccatccatcagcaacagcacaactatgctgcccgttttcctccagaaagctgctcccaagaagaggcttcagagaggggtccccaagaagaggttctggagagagatgctgcaggggaaaaggaagatgaggaggatgaagagattgtgagtcccccacctgtagaaagtgaggctgcccagtcctgccaccccaaacctgtcagttctgatactgaggatgtgaccaagaggaagaatcacaacttcctggagcgcaagaggcggaatgacctgcgttcgcgattcttggcgctgagggaccaggtgcccaccctggccagctgctccaaggcccccaaagtagtgatcctaagcaaggccttggaatacttgcaagccctggtgggggctgagaagaggatggctacagagaaaagacagctccgatgccggcagcagcagttgcagaaaagaattgcatacctcactggctacggagatctcaaaattgtcgctcctgtcaaacaaactcttaactttgatttactcaaactggctggggatgtagaaagcaatccaggtccactcatgaacaattcgcccttcaccatgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggca

SEQ ID NO: 10 - protein, 209 aa, Homo sapiens
MGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 11 - DNA, 47 bp, Homo sapiens
  source 1..47: mol_type = genomic DNA
gactccagtggtaatctacttcaagagagtagattaccactggagtc

SEQ ID NO: 12 - DNA, 11681 bp, synthetic construct
  source 1..11681: mol_type = other DNA
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcgggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgcccttcaccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaactgaggtaagggcgaattcaagcttcggggactagtcatatgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggtaaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatctcaattccgatcatattcaataacccttaatataacttcgtataatgtatgctatacgaagttattaggtctgaagaggagtttacgtccagccaagcttaggatcaattctcatgtttgacagcttatcatcgataagctgatcctcacaggccgcacccagcttttcttccgttgccccagtagcatctctgtctggtgaccttgaagaggaagaggaggggtcccgagaatccccatccctaccgtccagcaaaaagggggacgaggaatttgaggcctggcttgaggctcaggacgcaaatcttgaggatgttcagcgggagttttccgggctgcgagtaattggtgatgaggacgaggatggttcggaggatggggaattttcagacctggatctgtctgacagcgaccatgaaggggatgagggtgggggggctgttggagggggcaggagtctgcactccctgtattcactgagcgtcgtctaataaagatgtctattgatctcttttagtgtgaatcatgtctgacgaggggccaggtacaggacctggaaatggcctaggagagaagggagacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaagggacccacggtggaacaggagcaggagcaggagcgggaggggcaggagcaggaggggcaggagcaggaggaggggcaggagcaggaggaggggcaggaggggcaggaggggcaggaggggcaggagcaggaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggggcaggaggggcaggaggggcaggagcaggaggggcaggagcaggaggaggggcaggaggggcaggaggggcaggagcaggaggggcaggagcaggaggggcaggagcaggaggggcaggagcaggaggggcaggaggggcaggagcaggaggggcaggaggggcaggagcaggaggggcaggaggggcaggagcaggaggaggggcaggaggggcaggagcaggaggaggggcaggaggggcaggagcaggaggggcaggaggggcaggagcaggaggggcaggaggggcaggagcaggaggggcaggaggggcaggagcaggaggaggggcaggagcaggaggggcaggagcaggaggtggaggccggggtcgaggaggcagtggaggccggggtcgaggaggtagtggaggccggggtcgaggaggtagtggaggccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaactgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcagggtgactgtgtgcagctttgacgatggagtagatttgcctccctggtttccacctatggtggaaggggctgccgcggagggtgatgacggagatgacggagatgaaggaggtgatggagatgagggtgaggaagggcaggagtgatgtaacttgttaggagacgccctcaatcgtattaaaagccgtgtattcccccgcactaaagaataaatccccagtagacatcatgcgtgctgttggtgtatttctggccatctgtcttgtcaccattttcgtcctcccaacatggggcaattgccggaacccttaatataacttcgtataatgtatgctatacgaagttattaggtccctcgaagaggttcactagcggatctcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaaggcaatgttgtgttgcagtccacagactgcaaagtctgctccaggatgaaagccactcagtgttggcaaatgtgcacatccatttataaggatgtcaactacagtcagagaacccctttgtgtttggtccccccccgtgtcacatgtggaacagggcccagttggcaagttgtaccaaccaactgaagggattacatgcactgccccgcgaagaaggggcagagatgtcgtagtcaggtttagttcgtccggggcggggcatcgatcctctagagtcgacgctagcggatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgggccgactccagtggtaatctacttcaagagagtagattaccactggagtcttttttggaattcctgcagcccgggggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 13 - DNA, 12693 bp, synthetic construct
  source 1..12693: mol_type = other DNA
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcgggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgcccttcaccatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgggggatctcaaaattgtcgctcctgtcaaacaaactcttaactttgatttactcaaactggctggggatgtagaaagcaatccaggtccactcatgaacaattcgcccttcaccatggctgtcagtgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagagacatttttaaaagggcgaattgcctgcaggaattcaagcttcggggactagtcatatgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggtaaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatctcaattccgatcatattcaataacccttaatataacttcgtataatgtatgctatacgaagttattaggtctgaagaggagtttacgtccagccaagcttaggatcaattctcatgtttgacagcttatcatcgataagctgatcctcacaggccgcacccagcttttcttccgttgccccagtagcatctctgtctggtgaccttgaagaggaagaggaggggtcccgagaatccccatccctaccgtccagcaaaaagggggacgaggaatttgaggcctggcttgaggctcaggacgcaaatcttgaggatgttcagcgggagttttccgggctgcgagtaattggtgatgaggacgaggatggttcggaggatggggaattttcagacctggatctgtctgacagcgaccatgaaggggatgagggtgggggggctgttggagggggcaggagtctgcactccctgtattcactgagcgtcgtctaataaagatgtctattgatctcttttagtgtgaatcatgtctgacgaggggccaggtacaggacctggaaatggcctaggagagaagggagacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaagggacccacggtggaacaggagcaggagcaggagcgggaggggcaggagcaggaggggcaggagcaggaggaggggcaggagcaggaggaggggcaggaggggcaggaggggcaggaggggcaggagcaggaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggggcaggaggggcaggaggggcaggagcaggaggggcaggagcaggaggaggggcaggaggggcaggaggggcaggagcaggaggggcaggagcaggaggggcaggagcaggaggggcaggagcaggaggggcaggaggggcaggagcaggaggggcaggaggggcaggagcaggaggggcaggaggggcaggagcaggaggaggggcaggaggggcaggagcaggaggaggggcaggaggggcaggagcaggaggggcaggaggggcaggagcaggaggggcaggaggggcaggagcaggaggggcaggaggggcaggagcaggaggaggggcaggagcaggaggggcaggagcaggaggtggaggccggggtcgaggaggcagtggaggccggggtcgaggaggtagtggaggccggggtcgaggaggtagtggaggccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaactgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcagggtgactgtgtgcagctttgacgatggagtagatttgcctccctggtttccacctatggtggaaggggctgccgcggagggtgatgacggagatgacggagatgaaggaggtgatggagatgagggtgaggaagggcaggagtgatgtaacttgttaggagacgccctcaatcgtattaaaagccgtgtattcccccgcactaaagaataaatccccagtagacatcatgcgtgctgttggtgtatttctggccatctgtcttgtcaccattttcgtcctcccaacatggggcaattgccggaacccttaatataacttcgtataatgtatgctatacgaagttattaggtccctcgaagaggttcactagcggatctcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaaggcaatgttgtgttgcagtccacagactgcaaagtctgctccaggatgaaagccactcagtgttggcaaatgtgcacatccatttataaggatgtcaactacagtcagagaacccctttgtgtttggtccccccccgtgtcacatgtggaacagggcccagttggcaagttgtaccaaccaactgaagggattacatgcactgccccgcgaagaaggggcagagatgtcgtagtcaggtttagttcgtccggggcggggcatcgatcctctagagtcgacgctagcggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 14 - DNA, 12051 bp, synthetic construct
  source 1..12051: mol_type = other DNA
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcgggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgcccttcaccatggactacgactcgtaccagcactatttctacgactatgactgcggggaggatttctaccgctccacggcgcccagcgaggacatctggaagaaattcgagctggtgccatcgccccccacgtcgccgccctggggcttgggtcccggcgcaggggacccggcccccgggattggtcccccggagccgtggcccggagggtgcaccggagacgaagcggaatcccggggccactcgaaaggctggggcaggaactacgcctccatcatacgccgtgactgcatgtggagcggcttctcggcccgggaacggctggagagagctgtgagcgaccggctcgctcctggcgcgccccgggggaacccgcccaaggcgtccgccgccccggactgcactcccagcctcgaagccggcaacccggcgcccgccgccccctgtccgctgggcgaacccaagacccaggcctgctccgggtccgagagcccaagcgactcggagaatgaagaaattgatgttgtgacagtagagaagaggcagtctctgggtattcggaagccggtcaccatcacggtgcgagcagaccccctggatccctgcatgaagcatttccacatctccatccatcagcaacagcacaactatgctgcccgttttcctccagaaagctgctcccaagaagaggcttcagagaggggtccccaagaagaggttctggagagagatgctgcaggggaaaaggaagatgaggaggatgaagagattgtgagtcccccacctgtagaaagtgaggctgcccagtcctgccaccccaaacctgtcagttctgatactgaggatgtgaccaagaggaagaatcacaacttcctggagcgcaagaggcggaatgacctgcgttcgcgattcttggcgctgagggaccaggtgcccaccctggccagctgctccaaggcccccaaagtagtgatcctaagcaaggccttggaatacttgcaagccctggtgggggctgagaagaggatggctacagagaaaagacagctccgatgccggcagcagcagttgcagaaaagaattgcatacctcactggctacggagatctcaaaattgtcgctcctgtcaaacaaactcttaactttgatttactcaaactggctggggatgtagaaagcaatccaggtccactcatgaacaattcgcccttcaccatgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaattgaaagggcgaattgcctgcaggaattcaagcttcggggactagtcatatgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggtaaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatctcaattccgatcatattcaataacccttaatataacttcgtataatgtatgctatacgaagttattaggtctgaagaggagtttacgtccagccaagcttaggatcaattctcatgtttgacagcttatcatcgataagctgatcctcacaggccgcacccagcttttcttccgttgccccagtagcatctctgtctggtgaccttgaagaggaagaggaggggtcccgagaatccccatccctaccgtccagcaaaaagggggacgaggaatttgaggcctggcttgaggctcaggacgcaaatcttgaggatgttcagcgggagttttccgggctgcgagtaattggtgatgaggacgaggatggttcggaggatggggaattttcagacctggatctgtctgacagcgaccatgaaggggatgagggtgggggggctgttggagggggcaggagtctgcactccctgtattcactgagcgtcgtctaataaagatgtctattgatctcttttagtgtgaatcatgtctgacgaggggccaggtacaggacctggaaatggcctaggagagaagggagacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaagggacccacggtggaacaggagcaggagcaggagcgggaggggcaggagcaggaggggcaggagcaggaggaggggcaggagcaggaggaggggcaggaggggcaggaggggcaggaggggcaggagcaggaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggggcaggaggggcaggaggggcaggagcaggaggggcaggagcaggaggaggggcaggaggggcaggaggggcaggagcaggaggggcaggagcaggaggggcaggagcaggaggggcaggagcaggaggggcaggaggggcaggagcaggaggggcaggaggggcaggagcaggaggggcaggaggggcaggagcaggaggaggggcaggaggggcaggagcaggaggaggggcaggaggggcaggagcaggaggggcaggaggggcaggagcaggaggggcaggaggggcaggagcaggaggggcaggaggggcaggagcaggaggaggggcaggagcaggaggggcaggagcaggaggtggaggccggggtcgaggaggcagtggaggccggggtcgaggaggtagtggaggccggggtcgaggaggtagtggaggccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaactgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcagggtgactgtgtgcagctttgacgatggagtagatttgcctccctggtttccacctatggtggaaggggctgccgcggagggtgatgacggagatgacggagatgaaggaggtgatggagatgagggtgaggaagggcaggagtgatgtaacttgttaggagacgccctcaatcgtattaaaagccgtgtattcccccgcactaaagaataaatccccagtagacatcatgcgtgctgttggtgtatttctggccatctgtcttgtcaccattttcgtcctcccaacatggggcaattgccggaacccttaatataacttcgtataatgtatgctatacgaagttattaggtccctcgaagaggttcactagcggatctcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaaggcaatgttgtgttgcagtccacagactgcaaagtctgctccaggatgaaagccactcagtgttggcaaatgtgcacatccatttataaggatgtcaactacagtcagagaacccctttgtgtttggtccccccccgtgtcacatgtggaacagggcccagttggcaagttgtaccaaccaactgaagggattacatgcactgccccgcgaagaaggggcagagatgtcgtagtcaggtttagttcgtccggggcggggcatcgatcctctagagtcgacgctagcggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 15 - DNA, 21 bp, synthetic construct
  source 1..21: mol_type = other DNA
aaggtcccggtcaagaaacag

SEQ ID NO: 16 - DNA, 20 bp, synthetic construct
  source 1..20: mol_type = other DNA
cttctgcgtcacaccattgc

SEQ ID NO: 17 - DNA, 20 bp, synthetic construct
  source 1..20: mol_type = other DNA
cccacatgaagcgacttccc

SEQ ID NO: 18 - DNA, 21 bp, synthetic construct
  source 1..21: mol_type = other DNA
aggtccaggagatcgttgaac

SEQ ID NO: 19 - DNA, 20 bp, synthetic construct
  source 1..20: mol_type = other DNA
tggacagttacgcgcacatg

SEQ ID NO: 20 - DNA, 21 bp, synthetic construct
  source 1..21: mol_type = other DNA
gagtaggacatgctgtaggtg

SEQ ID NO: 21 - DNA, 20 bp, synthetic construct
  source 1..20: mol_type = other DNA
tgcggcccttgctgcagaag

SEQ ID NO: 22 - DNA, 20 bp, synthetic construct
  source 1..20: mol_type = other DNA
gctgctgggcgatgtggctg

SEQ ID NO: 23 - DNA, 22 bp, synthetic construct
  source 1..22: mol_type = other DNA
atgcattcaaactgaggtaagg

SEQ ID NO: 24 - DNA, 22 bp, synthetic construct
  source 1..22: mol_type = other DNA
tagcgtaaaaggagcaacatag

SEQ ID NO: 25 - DNA, 21 bp, synthetic construct
  source 1..21: mol_type = other DNA
ccatatggtagcctcatgtcc

SEQ ID NO: 26 - DNA, 21 bp, synthetic construct
  source 1..21: mol_type = other DNA
tcaattctgtgcctccgggag

SEQ ID NO: 27 - DNA, 23 bp, synthetic construct
  source 1..23: mol_type = other DNA
accacctcgccttacacatgaag

SEQ ID NO: 28 - DNA, 22 bp, synthetic construct
  source 1..22: mol_type = other DNA
tagcgtaaaaggagcaacatag

SEQ ID NO: 29 - DNA, 21 bp, synthetic construct
  source 1..21: mol_type = other DNA
ggctgagaagaggatggctac

SEQ ID NO: 30 - DNA, 21 bp, synthetic construct
  source 1..21: mol_type = other DNA
agtttgtttgacaggagcgac

SEQ ID NO: 31 - DNA, 20 bp, synthetic construct
  source 1..20: mol_type = other DNA
tcacatgtcccagcactacc

SEQ ID NO: 32 - DNA, 21 bp, synthetic construct
  source 1..21: mol_type = other DNA
agtttgtttgacaggagcgac

SEQ ID NO: 33 - DNA, 20 bp, synthetic construct
  source 1..20: mol_type = other DNA
aacccaagacccaggcctgc

SEQ ID NO: 34 - DNA, 20 bp, synthetic construct
  source 1..20: mol_type = other DNA
ggtctgctcgcaccgtgatg

SEQ ID NO: 35 - DNA, 20 bp, synthetic construct
  source 1..20: mol_type = other DNA
gaaatggcctaggagagaag

SEQ ID NO: 36 - DNA, 20 bp, synthetic construct
  source 1..20: mol_type = other DNA
cagccaatgcaacttggacg

SEQ ID NO: 37 - DNA, 24 bp, synthetic construct
  source 1..24: mol_type = other DNA
cttatgctacgtaaaggagctggg

SEQ ID NO: 38 - DNA, 24 bp, synthetic construct
  source 1..24: mol_type = other DNA
ttgtgccaacccaggtcccggaag

SEQ ID NO: 39 - DNA, 24 bp, synthetic construct
  source 1..24: mol_type = other DNA
tatcagatcctaaacagctcgcag

SEQ ID NO: 40 - DNA, 23 bp, synthetic construct
  source 1..23: mol_type = other DNA
cgtacgcaaattaaagtccagag

SEQ ID NO: 41 - DNA, 23 bp, synthetic construct
  source 1..23: mol_type = other DNA
actacaacgcctacgagtcctac

SEQ ID NO: 42 - DNA, 24 bp, synthetic construct
  source 1..24: mol_type = other DNA
gttgcaccagaaaagtcagagttg

SEQ ID NO: 43 - DNA, 24 bp, synthetic construct
  source 1..24: mol_type = other DNA
atatcccgccgtgggtgaaagttc

SEQ ID NO: 44 - DNA, 24 bp, synthetic construct
  source 1..24: mol_type = other DNA
actcagccatggactggagcatcc

SEQ ID NO: 45 - DNA, 26 bp, synthetic construct
  source 1..26: mol_type = other DNA
cctgctcaagctgactcgacaccgtg

SEQ ID NO: 46 - DNA, 25 bp, synthetic construct
  source 1..25: mol_type = other DNA
ggaaaagctggccctggggtggagc

SEQ ID NO: 47 - DNA, 22 bp, synthetic construct
  source 1..22: mol_type = other DNA
tgctgctcacagggcccgatac

SEQ ID NO: 48 - DNA, 23 bp, synthetic construct
  source 1..23: mol_type = other DNA
tcctttcgagctcagtgcaccac

SEQ ID NO: 49 - DNA, 21 bp, synthetic construct
  source 1..21: mol_type = other DNA
ctggcgctgagtacgtcgtgg

SEQ ID NO: 50 - DNA, 21 bp, synthetic construct
  source 1..21: mol_type = other DNA
gcagttggtggtgcaggaggc